(19) United States
(12) Patent Application Publication
(10) Pub. No.: US 2023/0193235 A1
(43) Pub. Date: Jun. 22, 2023

(54) MODIFIED ANGIOTENSIN-CONVERTING ENZYME 2 (ACE2) AND USE THEREOF

(71) Applicant: The Board of Trustees of the University of Illinois, Urbana, IL (US)

(72) Inventors: Erik Procko, Champaign, IL (US); Asrar Malik, Chicago, IL (US); Jalees Rehman, Chicago, IL (US); Lianghui Zhang, Chicago, IL (US); Shiqin Xiong, Chicago, IL (US)

(73) Assignee: The Board of Trustees of the University of Illinois, Urbana, IL (US)

(21) Appl. No.: 17/911,813

(22) PCT Filed: Mar. 16, 2021
(86) PCT No.: PCT/US2021/022611

§ 371 Date: Sep. 15, 2022

Related U.S. Application Data
(60) Provisional application No. 63/089,895, filed on Oct. 9, 2020.
(60) Provisional application No. 63/042,907, filed on Jun. 23, 2020.
(60) Provisional application No. 63/022,151, filed on May 8, 2020.
(60) Provisional application No. 62/989,976, filed on Mar. 16, 2020.

(51) Int. Cl. C12N 9/64; G01N 33/569; A61P 31/14; C12N 15/62

(52) U.S. Cl. CPC C12N 9/6421; C12Y 304/17023; G01N 33/56983; A61P 31/14; C12N 15/62

(57) ABSTRACT

The modified polypeptides include at least one amino acid substitution that allows the polypeptide to bind better to the S surface glycoprotein of coronaviruses that use ACE2 as a cell entry receptor, either through direct increases in affinity or through improved folding and expression of ACE2. Use of the modified ACE2 polypeptides for inhibiting CoV entry, replication and/or spread, for pre-exposure and post-exposure CoV prophylaxis, and for treating a CoV infection (e.g. COVTD-19), is also described.

### CROSS REFERENCE TO RELATED APPLICATIONS

This application claims the benefit of U.S. Provisional Application No. 63/089,895, filed Oct. 9, 2020, U.S. Provisional Application No. 63/042,907, filed Jun. 23, 2020, U.S. Provisional Application No. 63/022,151, filed May 8, 2020 and U.S. Provisional Application No. 62/989,976, filed Mar. 16, 2020, each of which is herein incorporated by reference in its entirety.

### ACKNOWLEDGMENT OF GOVERNMENT SUPPORT

This invention was made with government support under grant number 5R01AI129719-03 awarded by the National Institutes of Health. The government has certain rights in the invention.

### FIELD

This disclosure concerns modified angiotensin-converting enzyme 2 (ACE2) proteins with enhanced folding and increased binding to SARS-CoV-2 and other coronaviruses that use ACE2 as a cell entry receptor.

### BACKGROUND

In December 2019, a novel zoonotic betacoronavirus closely related to bat coronaviruses spilled over to humans at the Huanan Seafood Market in the Chinese city of Wuhan (Zhu et al., N Engl J Med. 2020 Feb 20;382(8):727-733; Zhou et al., Nature. 2020 Feb 3;579(7798):270-273). The virus, called SARS-CoV-2 due to its similarities with the severe acute respiratory syndrome (SARS) coronavirus responsible for a smaller outbreak nearly two decades prior (Peiris et al., Lancet. 2003 Apr 19;361(9366):1319-1325; Coronaviridae Study Group of the International Committee on Taxonomy of Viruses, Nat Microbiol. 2020 Mar 2;4(5):3), has since spread human-to-human rapidly across the world, precipitating extraordinary containment measures from governments (Patel et al., MMWR Morb Mortal Wkly Rep. 2020 Feb 7;69(5):140-146). Stock markets have fallen, travel restrictions have been imposed, public gatherings canceled, and large numbers of people are quarantined. These events are unlike any experienced in generations. Symptoms of coronavirus disease 2019 (COVID-19) range from mild to dry cough, fever, pneumonia and death, and SARS-CoV-2 is devastating among the elderly and other vulnerable groups (Wang et al, J Med Virol. 2020 April; 92(4):441-447; Huang et al., Lancet. 2020 Feb 15;395(10223):497-506). There is currently no vaccine to prevent infection with SARS-CoV-2 and no approved drugs to specifically treat infection with this virus. Thus, a need exists for the development of effective therapies to treat SARS-CoV-2 infection.

### SUMMARY

Described herein are human ACE2 polypeptides that exhibit enhanced binding to the S protein of SARS-CoV-2, either through enhanced folding and structural stabilization of ACE2, elimination of a glycan modification, or increased affinity. The modified polypeptides can be used as diagnostic or therapeutic agents for the detection, prophylaxis (pre- or post-exposure prophylaxis), or treatment of COVID-19, or disease caused by any coronavirus that utilizes ACE2 as a cellular receptor.

Provided herein are modified ACE2 polypeptides that include an ACE2 or a fragment thereof, such as an extracellular fragment. The polypeptides include at least one amino acid substitution relative to wild-type ACE2, and have increased capability to bind coronavirus S, either directly due to changes in affinity, or indirectly (for example, through stabilization of S-recognized structure). In particular examples, the ACE2 is a human ACE2. In some embodiments, the at least one amino acid substitution is selected from any of the substitutions shown in Table 1, Table 2 and/or Table 3. In some examples, the at least one amino acid substitution is a residue located at the interface of ACE2 and S. In some examples, the at least one amino acid substitution is a residue located in the N90-glycosylation motif. In some examples, the at least one amino acid substitution is distal from the interface and enhances presentation of S-recognized folded structure. In some examples, the modified ACE2 polypeptides are dimeric. In one example, the dimeric ACE2 comprises the T27Y, L79T, and N330Y amino acid substitutions.

Also provided herein are fusion proteins that include a modified ACE2 polypeptide disclosed herein and a heterologous polypeptide. In some embodiments, the heterologous polypeptide is an Fc protein or human serum albumin, such as for recruitment of effector functions and/or increased serum stability. In some embodiments, the heterologous polypeptide is a protein that can be used as a diagnostic/detection reagent, such as a fluorescent protein (for example, GFP) or an enzyme (for example, horseradish peroxidase (HRP) or alkaline phosphatase).

Further provided is a method of inhibiting coronavirus cell entry by contacting the virus with a modified ACE2 polypeptide or fusion protein disclosed herein. Methods of inhibiting coronavirus replication and/or spread in a subject are also provided. In some embodiments, the method includes administering to the subject a therapeutically effective amount of a modified ACE2 polypeptide or fusion protein disclosed herein. The modified ACE2 polypeptide can be administered prior to infection (such as in a subject at risk for infection) as a pre-exposure prophylactic treatment, shortly after infection as a post-exposure prophylactic, or after a subject exhibits one or more signs or symptoms of infection.

Also provided is a method of treating a coronavirus infection (e.g. COVID-19) in a subject by administering to the subject a therapeutically effective amount of a modified ACE2 polypeptide or fusion protein disclosed herein. The coronavirus can be any human or zoonotic coronavirus, including emerging strains of coronavirus, that utilize ACE2 as a cell entry receptor. In some examples, the modified ACE2 polypeptide is administered intravenously, intratracheally or via inhalation. The treatment method can be a pre-exposure prophylactic treatment method, a post-exposure prophylactic treatment method or a method of treating COVID-19.

Also provided are nucleic acid molecules and vectors that encode a modified ACE2 polypeptide or fusion protein disclosed herein. Methods of inhibiting CoV replication and/or spread (or treating a CoV infection) in a subject by administering the nucleic acid molecule or vector are further provided. In some examples, the nucleic acid molecule or vector is administered intravenously, intratracheally or via inhalation.

Further provided are methods of detecting a CoV in a biological sample. In some embodiments, the method includes contacting the biological sample with a modified polypeptide or fusion protein disclosed herein; and detecting binding of the modified polypeptide or fusion protein to the biological sample.

Also provided are kits that include a modified polypeptide or fusion protein disclosed herein bound to a solid support.

The foregoing and other objects and features of the disclosure will become more apparent from the following detailed description, which proceeds with reference to the accompanying figures.

### BRIEF DESCRIPTION OF THE DRAWINGS

FIGS. 1A-1D: A selection strategy for ACE2 variants with high binding to the RBD of SARS-CoV-2 S. (FIG. 1A) Media from Expi293F cells secreting the SARS-CoV-2 S-RBD fused to sfGFP was collected and incubated at different dilutions with Expi293F cells expressing myc-tagged ACE2. Bound S-RBD-sfGFP was measured by flow cytometry. The dilutions of S-RBD-sfGFP-containing medium used for FACS selections are indicated by arrows. (FIGS. 1B-1C) Expi293F cells were transiently transfected with wild type ACE2 plasmid diluted with a large excess of carrier DNA. Under these conditions, cells typically acquire no more than one coding plasmid and most cells are negative. Cells were incubated with S-RBD-sfGFP-containing medium and co-stained with fluorescent anti-myc to detect surface ACE2 by flow cytometry. During analysis, the top 67% were chosen from the ACE2-positive population (FIG. 1). Bound S-RBD was subsequently measured relative to surface ACE2 expression (FIG. 1C). (FIG. 1D) Expi293F cells were transfected with an ACE2 single site-saturation mutagenesis library and analyzed as in FIG. 1B. During FACS, the top 15% of cells with bound S-RBD relative to ACE2 expression were collected (nCoV-S-High sort) and the bottom 20% were collected separately (nCoV-S-Low sort).

FIG. 2: A mutational landscape of ACE2 for high binding signal to the RBD of SARS-CoV-2 S. Log2 enrichment ratios from the nCoV-S-High sorts are plotted from ≤−3 (i.e. depleted/deleterious) to neutral to ≥+3 (i.e. enriched). ACE2 primary structure is on the vertical axis, amino acid substitutions are on the horizontal axis. *, stop codon.

FIGS. 3A-3F: Data from independent replicates show close agreement. (FIGS. 3A-3B) Log2 enrichment ratios for ACE2 mutations in the nCoV-S-High (FIG. 3A) and nCoV-S-Low (FIG. 3B) sorts closely agree between two independent FACS experiments. Replicate 1 used a 1/40 dilution and replicate 2 used a 1/20 dilution of S-RBD-sfGFP-containing medium. R² values are for nonsynonymous mutations. (FIG. 3C) Average log 2 enrichment ratios tend to be anticorrelated between the nCoV-S-High and nCoV-S-Low sorts. Nonsense mutations and a small number of nonsynonymous mutations are not expressed at the plasma membrane and are depleted from both sort populations (i.e. fall below the diagonal). (FIGS. 3D-3F) Correlation plots of residue conservation scores from replicate nCoV-S-High (FIG. 3D) and nCoV-S-Low (FIG. 3E) sorts, and from the averaged data from both nCoV-S-High sorts compared to both nCoV-S-Low sorts (FIG. 3F). Conservation scores are calculated from the mean of the log 2 enrichment ratios for all amino acid substitutions at each residue position.

FIGS. 4A-4C: Sequence preferences of ACE2 residues for high binding to the RBD of SARS-CoV-2 S. (FIG. 4A) Conservation scores from the nCoV-S-High sorts are mapped to the cryo-EM structure (PDB 6M17) of S-RBD bound ACE2 (surface). The view at left is looking down the substrate-binding cavity, and only a single protease domain is shown for clarity. (FIG. 4B) Average hydrophobicity-weighted enrichment ratios are mapped to the RBD-bound ACE2 structure. (FIG. 4C) A magnified view of part of the ACE2. Accompanying heatmap plots log 2 enrichment ratios from the nCoV-S-High sort for substitutions of ACE2-T27, D30 and K31 from ≤−3 (depleted) to ≥+3 (enriched).

FIGS. 5A-5C: Single amino acid substitutions in ACE2 predicted from the deep mutational scan to increase RBD binding have small effects. (FIG. 5A) Expi293F cells expressing full length ACE2 were stained with RBD-sfGFP-containing medium and analyzed by flow cytometry. Data are compared between wild type ACE2 and a single mutant (L79T). Increased RBD binding is most discernable in cells expressing low levels of ACE2 (smaller gate). In this experiment, ACE2 has an extracellular N-terminal myc tag upstream of residue S19 that is used to detect surface expression. (FIG. 5B) RBD-sfGFP binding was measured for 30 amino acid substitutions in ACE2. Data are the relative change in GFP mean fluorescence in the low expression gate with background fluorescence subtracted. Mean of n=2, error bars represent range. (FIG. 5C) Relative RBD-sfGFP binding measured for the total ACE2-positive population (larger gate in FIG. 5A) is shown in the upper graph, while the lower graph plots relative ACE2 expression measured by detection of the extracellular myc tag. RBD-sfGFP binding to the total positive population correlates with total ACE2 expression, and differences in binding between the mutants are therefore most apparent only after controlling for expression levels as in FIG. 5B.

FIGS. 6A-6B: Engineered sACE2 with enhanced binding to S. (FIG. 6A) Expression of sACE2-sfGFP mutants was qualitatively evaluated by fluorescence of the transfected cell cultures. (FIG. 6B) Cells expressing full length S were stained with dilutions of sACE2-sfGFP-containing media and binding was analyzed by flow cytometry.

FIGS. 7A-7D: Analytical size exclusion chromatography (SEC) of purified sACE2 proteins. (FIG. 7A) Purified sACE2 proteins (10 μg) were separated on a 4-20% SDS-polyacrylamide gel and stained with Coomassie. (FIG. 7B) Analytical SEC of IgG1-fused wild type sACE2 and sACE2.v2. Molecular weights (MW) of standards are indicated in kD above the peaks. Absorbance of the MW standards is scaled for clarity. (FIG. 7C) Analytical SEC of 8his-tagged proteins. The major peak corresponds to the expected MW of a monomer. A dimer peak is also observed, although its abundance differs between independent protein preparations (compare to FIG. 9D). (FIG. 7D) Soluble ACE2-8h proteins were incubated at 37° C. for 40 h and analyzed by SEC.

FIGS. 8A-8E: A variant of sACE2 with high affinity for S. (FIG. 8A) Expi293F cells expressing full length S were incubated with purified wild type sACE2 or sACE2.v2 fused to 8his (solid lines) or IgG1-Fc (broken lines). After washing, bound protein was detected by flow cytometry. (FIG. 8B) Binding of 100 nM wild type sACE2-IgG1 (broken lines) was competed with wild type sACE2-8h or sACE2.v2-8h. The competing proteins were added simultaneously to cells expressing full length S, and bound proteins were detected by flow cytometry. (FIG. 8C) Biolayer interferometry (BLI) kinetics of wild type sACE2-8h association (t=0 to 120 s) and dissociation (t>120 s) with immobilized RBD-IgG1. (FIG. 8D) Kinetics of sACE2.v2-8h binding to immobilized RBD-IgG1 measured by BLI. (FIG. 8E) Competition for binding to immobilized RBD in an ELISA between serum IgG from recovered COVID-19 patients versus wild type sACE2-8h or sACE2.v2-8h. Three different patient sera were tested (P1 to P3 in light to dark shades).

FIGS. 9A-9G: Optimization of a high affinity sACE2 variant for improved yield. (FIG. 9A) Dilutions of sACE2-sfGFP-containing media were incubated with Expi293F cells expressing full length S. After washing, bound sACE2-sfGFP was analyzed by flow cytometry. (FIG. 9B) Coomassie-stained SDS-polyacrylamide gel compares the yield of sACE2-IgG1 variants purified from expression medium by protein A resin. (FIG. 9C) Coomassie-stained gel of purified sACE2-8h variants (10 μg per lane). (FIG. 9D) By analytical SEC, sACE2.v2.4-8h is indistinguishable from wild type sACE2-8h. The absorbance of MW standards is scaled for clarity, with MW indicated above the elution peaks in kD. (FIG. 9E) Analytical SEC after storage at 37° C. for 60 h. Variant sACE2.v2.2 has a more hydrophobic surface and higher propensity to partially aggregate compared to sACE2.v2.4, and therefore the partial storage instability may be intrinsically linked to increased hydrophobicity. (FIG. 9F) Wild type sACE2-8h association (t=0 to 120 s) and dissociation (t>120 s) with immobilized RBD-IgG1 measured by BLI. Data are comparable to a second independent preparation of sACE2-8h shown in FIG. 8C. (FIG. 9G) BLI kinetics of sACE2.v2.4-8h with immobilized RBD-IgG1.

FIGS. 10A-10D: A dimeric sACE2 variant with improved properties for binding viral spike. (FIG. 10A) Analytical SEC of wild type sACE2₂-8h and sACE2₂.v2.4-8h after incubation at 37° C. for 62 h. (FIG. 10B) ELISA analysis of serum IgG from recovered patients (P1 to P3 in light to dark shades) binding to RBD. Dimeric sACE2₂(WT)-8h or sACE2₂.v2.4-8h are added to compete with antibodies recognizing the receptor binding site. Concentrations are based on monomeric subunits. (FIG. 10C) RBD-8h association (t=0 to 120 s) and dissociation (t>120 s) with immobilized sACE2₂(WT)-IgG1 measured by BLI. (FIG. 10D) BLI kinetics of RBD-8h binding to immobilized sACE2₂.v2.4-IgG1.

FIG. 11: Enhanced neutralization of SARS-CoV-2 and SARS-CoV-1 by engineered receptors. In a microneutralization assay, monomeric (solid lines) or dimeric (broken lines) sACE2(WT)-8h or sACE2.v2.4-8h were preincubated with virus before adding to VeroE6 cells. Concentrations are based on monomeric subunits. Data are mean±SEM of n=4 (2 independent experiments with 2 technical replicates).

FIGS. 12A-12C: Binding of a sACE2 glycosylation mutant to the RBD of SARS-CoV-2. (FIG. 12A) The protease domain of soluble ACE2 carrying mutation T92Q was purified as a 8his-tagged fusion. Six μg was separated on a Coomassie-stained 4-20% SDS-polyacrylamide gel to assess purity. (FIG. 12B) Analytical SEC shows a major peak eluting as monomer, with a smaller fraction eluting at the expected MW of dimer. (FIG. 12C) In biolayer interferometry (BLI) experiments, RBD-IgG1 was immobilized to a sensor surface and association and dissociation of sACE2-T92Q-8h was measured. The reported affinity (80 nM) is tighter than that of the equivalent wild type protein (140-150 nM, FIGS. 8C and 9F).

FIGS. 13A-13C: Flow cytometry measurements of sACE2 binding to myc-tagged S expressed at the plasma membrane. (FIG. 13A) Expi293F cells expressing full length S, either untagged (FIG. 8A) or with an extracellular myc epitope tag, were gated by forward-side scattering properties for the main cell population (gated area). (FIG. 13B) Histograms showing representative raw data from flow cytometry analysis of myc-S-expressing cells incubated with 200 nM wild type sACE2-8h or sACE2.v2. After washing, bound protein was detected with a fluorescent anti-HIS-FITC secondary. Fluorescence of myc-S-expressing cells treated without sACE2 is black. (FIG. 13C) Binding of purified wild type sACE2 or sACE2.v2 fused to 8his (solid lines) or IgG1-Fc (broken lines) to cells expressing myc-S.

FIGS. 14A-14D: Dimeric sACE2₂ binds avidly to RBD. (FIG. 14A) SDS-PAGE of purified dimeric sACE2₂-8h proteins (10 μg per lane, stained with Coomassie). (FIG. 14B) Preparative SEC of sACE2₂-8h proteins. The eluate from NiNTA affinity chromatography was concentrated and injected on the gel filtration column. Absorbance of MW standards is scaled and kD is indicated above the elution peaks. (FIG. 14C) Expi293F cells expressing full length S were incubated with wild type and v2.4 sACE2₂-8h, washed and stained with fluorescent anti-his. Mean of n=2, error bars represent range. Binding resembles that observed for IgG1-fusions of sACE2 (FIGS. 8A and 13C), indicating avid interactions between dimeric sACE2₂-8h and trimeric spike on the cell surface. (FIG. 14D) BLI kinetics for dimeric sACE2₂(WT)-8h and sACE2₂.v2.4-8h binding avidly to dimeric RBD-IgG1 immobilized on the sensor surface.

FIGS. 15A-15B: Purified sACE2₂-IgG1 is a dimer. (FIG. 15A) Coomassie-stained gel of purified sACE2₂-IgG1 proteins (10 μg per lane). (FIG. 15B) Analytical SEC of purified sACE2₂-IgG1, overlaid with scaled absorbance of MW standards (kD indicated above elution peaks). Note the absence of high MW peaks that might correspond to concatemers mediated by sACE2₂ and IgG1 dimerization between different subunits.

FIGS. 16A-16D: Untagged sACE2₂.v2.4 expressed in nonhuman cells binds S tightly. (FIG. 16A) SDS-PAGE comparison of sACE2₂.v2.4 purified from human Expi293F cells with a 8h tag and untagged protein manufactured in the nonhuman ExpiCHO-S line. 10 μg per lane. (FIG. 16B) Analytical SEC of sACE2₂.v2.4 before and after incubation at 37° C. for 146 h. Absorbance of MW standards is scaled and kD is indicated. (FIG. 16C)S-expressing Expi293F cells were co-incubated with 100 nM wild type sACE2₂-IgG1 and increasing concentrations of human cell-derived sACE2₂(WT)-8h, human cell-derived sACE2₂.v2.4-8h or ExpiCHO-S-derived sACE2₂.v2.4. Bound his-tagged proteins (solid lines) and sACE2₂(WT)-IgG1 (broken lines) were measured by flow cytometry. (FIG. 16D) Avid binding of untagged sACE2₂.v2.4 to immobilized RBD-IgG1 measured by BLI.

FIG. 17: The engineered receptor has reduced catalytic activity. Cleavage of a peptide substrate to release a fluorescent product was measured for human cell-derived sACE2₂(WT)-8h (left), human cell-derived sACE2₂.v2.4-8h (middle) and ExpiCHO-S-derived sACE2₂.v2.4 (right). Specific activity was determined across initial time points where product release was linear, and data for the highest concentration of wild type protein (22 nM) was excluded. Time=0 min indicates when fluorescence recording began.

FIG. 18: SARS-associated coronaviruses have high sequence diversity at the ACE2-binding site. The RBD of SARS-CoV-2 (PDB 6M17) is colored by diversity between 7 SARS-associated CoV strains.

FIG. 19: The ACE2-binding site of SARS-associated betacoronaviruses is a region of high sequence diversity. RBD sequences from 2 human and 5 bat betacoronaviruses that use ACE2 as an entry receptor are aligned (SEQ ID NOs: 3-9). Numbering is based on SARS-CoV-2 protein S. Asterisks indicate residues of SARS-CoV-2 RBD that are within 6.0 Å of ACE2 in PDB 6M17.

FIGS. 20A-20C: FACS selection for variants of S with high or low binding signal to ACE2. (FIG. 20A) Flow cytometry analysis of Expi293F cells expressing full-length S of SARS-CoV-2 with an N-terminal c-myc tag. Staining for the myc-epitope is on the x-axis while the detection of bound sACE2₂-8h (2.5 nM) is on the y-axis. S plasmid was diluted 1500-fold by weight with carrier DNA so that cells typically express no more than one coding variant; under these conditions most cells are negative. (FIG. 20B) Flow cytometry of cells transfected with the RBD single site-saturation mutagenesis (SSM) library shows cells expressing S variants with reduced sACE2₂-8h binding. (FIG. 20C) Gating strategy for FACS. S-expressing cells positive for the c-myc epitope were gated and the highest (“ACE2-High”) and lowest (“ACE2-Low”) 20% of cells with bound sACE2₂-8h relative to myc-S expression were collected.

FIG. 21: The mutational landscape across the RBD of full-length S from SARS-CoV-2 for binding to soluble ACE2₂. Log2 enrichment ratios from the deep mutational scan of the RBD in full-length S are plotted from ≤−3 (depleted/deleterious) to 0 (neutral) to ≥+3 (enriched). Wild type amino acids are black. RBD sequence is on the vertical axis and amino acid substitutions are on the horizontal axis. *, stop codons.

FIGS. 22A-22D: Deep mutagenesis reveals that the ACE2-binding site of SARS-CoV-2 tolerates many mutations. (FIG. 22A) Positional scores for surface expression are mapped to the structure of the SARS-CoV-2 RBD (PDB 6M17, oriented as in FIG. 18). Several residues in the protein core are highly conserved in the FACS selection for surface S expression (judged by depletion of mutations from the ACE2-High and ACE2-Low gates), while some surface residues tolerate mutations. (FIG. 22B) Correlation plot of expression scores from mutant selection in human cells of full-length S (x-axis) versus the conservation scores (mean of the log 2 enrichment ratios at a residue position) from mutant selection in the isolated RBD by yeast display (y-axis). Notable outliers are indicated. (FIG. 22C) Conservation scores from the ACE2-High gated cell population are mapped to the RBD structure. (FIG. 22D) Correlation plot of RBD conservation scores for high ACE2 binding from deep mutagenesis of S in human cells (x-axis) versus deep mutagenesis of the RBD on the yeast surface (mean of ΔK_{D app}; y-axis).

FIGS. 23A-23C: Alanine substitutions of disulfide-bonded cysteines in the RBD diminish S surface expression in human cells. (FIG. 23A) The RBD, colored by expression score from deep mutagenesis (conserved or mutationally tolerant), forms a continuous hydrophobic core with the rest of the S1 subunit in a closed-down conformation (PDB 6VSB chain B). (FIG. 23B) Based on surface immuno-staining and flow cytometry analysis, Expi293F cells transfected with myc-S cysteine mutants displayed decreases in both the percent of myc-positive cells (gated area) and in mean fluorescence of the positive population. To capture both effects in a single number, the change in mean fluorescence units (AMFU) compared to vector-transfected control cells was calculated for the entire cell population, after first gating by scattering for viable cells. (FIG. 23C) Surface expression of myc-S cysteine mutants relative to wild type myc-S. Data are mean±range, n=2 independent replicates.

FIGS. 24A-24G: A competition-based selection to identify RBD mutations within S of SARS-CoV-2 that preferentially bind wild type or engineered ACE2 receptors. (FIG. 24A) Expi293F cells were transfected with wild type myc-S and incubated with competing sACE2₂(WT)-IgG1 (25 nM) and sACE2₂.v2.4-8h (20 nM). Bound protein was detected by flow cytometry after immuno-staining for the respective epitope tags. (FIG. 24B) As in FIG. 24A, except cells were transfected with the RBD SSM library. A population of cells expressing S variants with increased specificity towards sACE2₂.v2.4 is apparent (cells shifted to the upper-left of the main population). (FIG. 24C) Gates used for FACS of cells expressing the RBD SSM library. After excluding cells without bound protein, the top 20% of cells for bound sACE2₂.v2.4-8h (upper gate) and for bound sACE2₂(WT)-IgG1 (lower gate) were collected. (FIGS. 24D-24E) Agreement between log 2 enrichment ratios from two independent FACS selections for cells expressing S variants with increased specificity for sACE2₂(WT) (FIG. 24D) or sACE2₂.v2.4 (FIG. 24E). R² values are calculated for nonsynonymous mutations. (FIGS. 24F-24G) Conservation scores are calculated from the mean of the log 2 enrichment ratios for all nonsynonymous substitutions at a given residue position. Correlation plots show agreement between conservation scores for two independent selections for cells within the sACE2₂(WT) (FIG. 24D) or sACE2₂.v2.4 (FIG. 24E) specific gates.

FIGS. 25A-25C: Mutations within the RBD that confer specificity towards wild type ACE2 are rare. (FIG. 25A) The SARS-CoV-2 RBD is colored by specificity score (the difference between the conservation scores for cells collected in the sACE2₂(WT) and sACE2₂.v2.4 specific gates). Some residues are hot spots for mutations with increased specificity towards sACE2₂(WT) or towards sACE2₂.v2.4. The contacting surface of ACE2 is shown as a ribbon, with sites of mutations in sACE2₂.v2.4 labeled and shown as spheres. (FIG. 25B) Log2 enrichment ratios for mutations in S expressed by cell populations collected in the sACE2₂(WT) (x-axis) and sACE2₂.v2.4 (y-axis) specific gates. Data are the mean from two independent sorting experiments. S mutants predicted to have increased specificity for sACE2₂(WT) were tested by targeted mutagenesis in FIG. 26. S mutants predicted to have increased specificity for sACE2₂.v2.4 were tested by targeted mutagenesis in FIG. 27. (FIG. 25C) Wild type myc-S and two variants, N501W and N501Y, were expressed in Expi293F cells and tested by flow cytometry for binding to sACE2₂(WT)-8h (dashed lines) or sACE2₂.v2.4-8h (solid lines). Minor increases in specific binding for wild type sACE2₂ are observed.

FIGS. 26A-26B: Screening mutations of SARS-CoV-2 S predicted by deep mutagenesis to have enhanced specificity towards wild type sACE2₂ over sACE2₂.v2.4. (FIG. 26A) Relative surface expression of myc-S mutants, determined as described in FIG. 23. Data are mean±range, n=2 independent replicates. (FIG. 26B) Competition binding between sACE2₂(WT)-IgG1 (x-axis) and sACE2₂.v2.4-8h (y-axis) on Expi293F cells expressing the indicated myc-tagged S proteins. Cells expressing S variants with increased specificity towards wild type receptor will be shifted to the lower-right; only minor shifts are observed. Cells expressing S variants with reduced surface expression and/or ACE2 affinity have a lower percentage in the gated area. Results are representative of 2 replicates.

FIGS. 27A-27B: Screening mutations of SARS-CoV-2 S predicted by deep mutagenesis to have enhanced specificity towards sACE2₂.v2.4 over wild type sACE2₂. (FIG. 27A) Relative surface expression of myc-S mutants measured by flow cytometry. Data are mean±range, n=2. (FIG. 28B) Flow cytometry analysis of cells expressing myc-S variants bound to competing sACE2₂(WT)-IgG1 (x-axis) and sACE2₂.v2.4-8h (y-axis). Cells expressing S with increased specificity towards sACE2₂.v2.4 will be shifted to the upper-left. Results are representative of 2 replicates.

FIGS. 28A-28B: Serum half-life of sACE2 peptides following IV administration. Unfused sACE2₂.v2.4 was injected in the tail veins of mice (3 male and 3 female per time point; 0.5 mg/kg). Serum was collected and analyzed by ACE2 ELISA (FIG. 28A) and for proteolytic activity towards a fluorogenic substrate (FIG. 28B). Data are mean±S.E.

FIG. 29: Pharmacokinetics of sACE2 fused to human IgG1 Fc following IV administration. IV administration of 2.0 mg/kg wild type sACE2₂-IgG1 (open circles) or sACE2₂.v2.4-IgG1 (filled circles) in 3 male mice per time point. Protein in serum was quantified by human IgG1 ELISA. Data are mean±S.E.

FIGS. 30A-30D: Pharmacokinetics of sACE2₂.v2.4-IgG1 in serum following IV administration. sACE2₂.v2.4-IgG1 was IV administered to mice (3 male and 3 female per time point; 2.0 mg/kg). Serum was collected and analyzed by human IgG1 ELISA (FIG. 30A), by ACE2 ELISA (FIG. 30B), and for ACE2 catalytic activity (FIG. 30C). Data are mean±S.E. (FIG. 30D) Serum samples from representative male mice were separated on a non-reducing SDS electrophoretic gel and probed with anti-human IgG1. The standard is 10 ng of purified sACE2₂.v2.4-IgG1. The predicted molecular weight (excluding glycans) is 216 kD.

FIGS. 31A-31F: PK of ACE2 proteins delivered directly to the lungs. (FIGS. 31A and 31B) Wild type sACE2₂-IgG1 (open circles) and sACE2₂.v2.4-IgG1 (filled circles) were administered IT at a dose of 1.0 mg/kg. Lung tissue was collected and proteins were extracted and analyzed by human IgG1 ELISA (FIG. 31A) and ACE2 ELISA (FIG. 31B). Data are mean S.E., n=3 males per time point. (FIG. 31C) Lung extracts from representative mice IT administered sACE2₂.v2.4-IgG1 were analyzed under non-reducing conditions by anti-human IgG1 immunoblot. (FIGS. 31D and 31E) Mice inhaled nebulized sACE2₂.v2.4-IgG1. Extracts from lung tissue were analyzed by ACE2 ELISA (FIG. 31D) and human IgG1 ELISA (FIG. 31E). Data are mean±S.E., n=3 males per time point. (FIG. 31F) Representative extracts from lung tissue of mice receiving nebulized sACE2₂.v2.4-IgG1 were analyzed by anti-human IgG1 immunoblot.

FIG. 32: Neutralization of pseudovirus entry into human lung cells by sACE2₂-IgG1. Human A549 lung epithelial cells over-expressing the ACE2 receptor, human A549 lung epithelial cells, and human lung endothelial cells were incubated with the VSV-SARS-CoV-2-luciferase-pseudotype virus and with wild-type sACE2₂-IgG1 or engineered sACE2₂.v2.4-IgG1 at the indicated concentrations. Each experiment contained a no virus control (left-most bar in each graph), all other samples contained the virus dose at the indicated MOI. The extent of viral entry was quantified based on luciferase activity.

FIG. 33: Efficacy of sACE2₂-IgG1 to inhibit pseudovirus entry into the lung and liver in an in vivo infection model. K18-hACE2 mice, which express the human ACE2 receptor in epithelial cells, were injected IV with sACE2₂-IgG1 (wild-type, middle bar; engineered v2.4, right bar) and intraperitoneally with the VSV-SARS-CoV-2-luciferase-pseudotype virus. The lung and the liver were harvested at 24 hours and the extent of viral entry was quantified based on luciferase expression.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file, created on Mar. 11, 2021, 43.7 KB, which is incorporated by reference herein. In the accompanying sequence listing:

SEQ ID NO: 1 is the amino acid sequence of human ACE2 (also called peptidyl-dipeptidase A; deposited under GenBank Accession No. NP 068576.1):

MSSSSWLLLSLVAVTAAQSTIEEQAKTFLDKFNHE
AEDLFYQSSLASWNYNTNITEENVQNMNNAGDKWS
AFLKEQSTLAQMYPLQEIQNLTVKLQLQALQQNGS
SVLSEDKSKRLNTILNTMSTIYSTGKVCNPDNPQE
CLLLEPGLNEIMANSLDYNERLWAWESWRSEVGKQ
LRPLYEEYVVLKNEMARANHYEDYGDYWRGDYEVN
GVDGYDYSRGQLIEDVEHTFEEIKPLYEHLHAYVR
AKLMNAYPSYISPIGCLPAHLLGDMWGRFWTNLYS
LTVPFGQKPNIDVTDAMVDQAWDAQRIFKEAEKFF
VSVGLPNMTQGFWENSMLTDPGNVQKAVCHPTAWD
LGKGDFRILMCTKVTMDDFLTAHHEMGHIQYDMAY
AAQPFLLRNGANEGFHEAVGEIMSLSAATPKHLKS
IGLLSPDFQEDNETEINFLLKQALTIVGTLPFTYM
LEKWRWMVFKGEIPKDQWMKKWWEMKREIVGVVEP
VPHDETYCDPASLFHVSNDYSFIRYYTRTLYQFQF
QEALCQAAKHEGPLHKCDISNSTEAGQKLFNMLRL
GKSEPWTLALENVVGAKNMNVRPLLNYFEPLFTWL
KDQNKNSFVGWSTDWSPYADQSIKVRISLKSALGD
KAYEWNDNEMYLFRSSVAYAMRQYFLKVKNQMILF
GEEDVRVANLKPRISFNFFVTAPKNVSDIIPRTEV
EKAIRMSRSRINDAFRLNDNSLEFLGIQPTLGPPN
QPPVSIWLIVFGVVMGVIVVGIVILIFTGIRDRKK
KNKARSGENPYASIDISKGENNPGFQNTDDVQTSF

SEQ ID NO: 2 is the amino acid sequence of the surface glycoprotein (protein S) of Severe acute respiratory syndrome coronavirus 2 (deposited under GenBank Accession No.

MFVFLVLLPLVSSQCVNLTTRTQLPPAYTNSFTRG
VYYPDKVFRSSVLHSTQDLFLPFFSNVTWFHAIHV
SGTNGTKRFDNPVLPFNDGVYFASTEKSNIIRGWI
FGTTLDSKTQSLLIVNNATNVVIKVCEFQFCNDPF
LGVYYHKNNKSWMESEFRVYSSANNCTFEYVSQPF
LMDLEGKQGNFKNLREFVFKNIDGYFKIYSKHTPI
NLVRDLPQGFSALEPLVDLPIGINITRFQTLLALH
RSYLTPGDSSSGWTAGAAAYYVGYLQPRTFLLKYN
ENGTITDAVDCALDPLSETKCTLKSFTVEKGIYQT
SNFRVQPTESIVRFPNITNLCPFGEVFNATRFASV
YAWNRKRISNCVADYSVLYNSASFSTFKCYGVSPT
KLNDLCFTNVYADSFVIRGDEVRQIAPGQTGKIAD
YNYKLPDDFTGCVIAWNSNNLDSKVGGNYNYLYRL
FRKSNLKPFERDISTEIYQAGSTPCNGVEGFNCYF
PLQSYGFQPTNGVGYQPYRVVVLSFELLHAPATVC
GPKKSTNLVKNKCVNFNFNGLTGTGVLTESNKKFL
PFQQFGRDIADTTDAVRDPQTLEILDITPCSFGGV
SVITPGTNTSNQVAVLYQDVNCTEVPVAIHADQLT
PTWRVYSTGSNVFQTRAGCLIGAEHVNNSYECDIP
IGAGICASYQTQTNSPRRARSVASQSIIAYTMSLG
AENSVAYSNNSIAIPTNFTISVTTEILPVSMTKTS
VDCTMYICGDSTECSNLLLQYGSFCTQLNRALTGI
AVEQDKNTQEVFAQVKQIYKTPPIKDFGGFNFSQI
LPDPSKPSKRSFIEDLLFNKVTLADAGFIKQYGDC
LGDIAARDLICAQKFNGLTVLPPLLTDEMIAQYTS
ALLAGTITSGWTFGAGAALQIPFAMQMAYRFNGIG
VTQNVLYENQKLIANQFNSAIGKIQDSLSSTASAL
GKLQDVVNQNAQALNTLVKQLSSNFGAISSVLNDI
LSRLDKVEAEVQIDRLITGRLQSLQTYVTQQLIRA
AEIRASANLAATKMSECVLGQSKRVDFCGKGYHLM
SFPQSAPHGVVFLHVTYVPAQEKNFTTAPAICHDG
KAHFPREGVFVSNGTHWFVTQRNFYEPQIITTDNT
FVSGNCDVVIGIVNNTVYDPLQPELDSFKEELDKY
FKNHTSPDVDLGDISGINASVVNIQKEIDRLNEVA
KNLNESLIDLQELGKYEQYIKWPWYIWLGFIAGLI
AIVMVTIMLCCMTSCCSCLKGCCSCGSCCKFDEDD
SEPVLKGVKLHYT

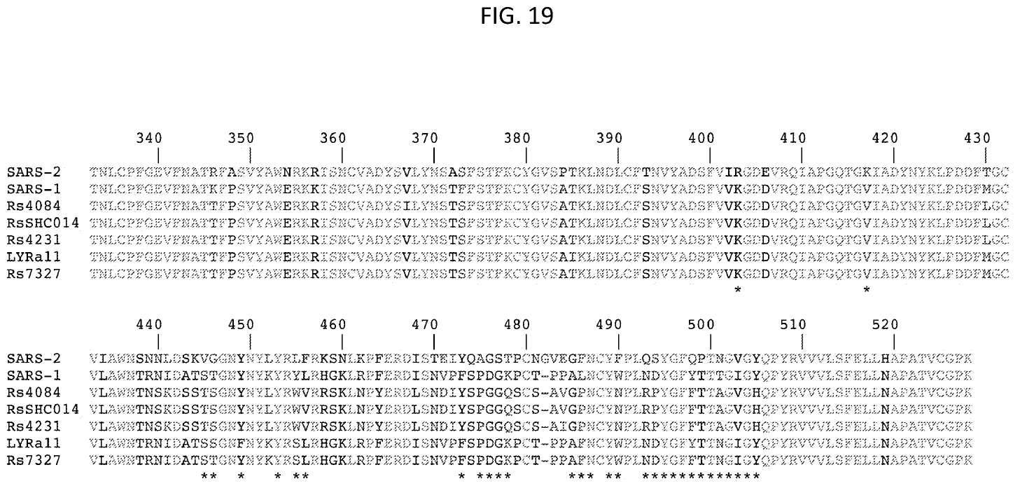

SEQ ID NOs: 3-9 are amino acid sequences of RBD sequences from human and bat betacoronaviruses (see FIG. 19).

SEQ ID NO: 10 is the amino acid sequence of sACE2₂.v2.4, comprised of residues 19-732 of human ACE2 (including the protease and dimerization domains) with three amino acid substitutions relative to human ACE2: T27Y, L79T, and N330Y.

STIEEQAKYFLDKFNHEAEDLFYQSSLASWNYNTN
ITEENVQNMNNAGDKWSAFLKEQSTTAQMYPLQEI
QNLTVKLQLQALQQNGSSVLSEDKSKRLNTILNTM
STIYSTGKVCNPDNPQECLLLEPGLNEIMANSLDY
NERLWAWESWRSEVGKQLRPLYEEYVVLKNEMARA
NHYEDYGDYWRGDYEVNGVDGYDYSRGQLIEDVEH
TFEEIKPLYEHLHAYVRAKLMNAYPSYISPIGCLP
AHLLGDMWGRFWTNLYSLTVPFGQKPNIDVTDAMV
DQAWDAQRIFKEAEKFFVSVGLPNMTQGFWEYSML
TDPGNVQKAVCHPTAWDLGKGDFRILMCTKVTMDD
FLTAHHEMGHIQYDMAYAAQPFLLRNGANEGFHEA
VGEIMSLSAATPKHLKSIGLLSPDFQEDNETEINF
LLKQALTIVGTLPFTYMLEKWRWMVFKGEIPKDQW
MKKWWEMKREIVGVVEPVPHDETYCDPASLFHVSN
DYSFIRYYTRTLYQFQFQEALCQAAKHEGPLHKCD
ISNSTEAGQKLFNMLRLGKSEPWTLALENVVGAKN
MNVRPLLNYFEPLFTWLKDQNKNSFVGWSTDWSPY
ADQSIKVRISLKSALGDKAYEWNDNEMYLFRSSVA
YAMRQYFLKVKNQMILFGEEDVRVANLKPRISFNF
FVTAPKNVSDIIPRTEVEKAIRMSRSRINDAFRLN
DNSLEFLGIQPTLG

SEQ ID NO: 11 is the amino acid sequence of sACE2₂.v2.4-IgG1, comprised of sACE2₂.v2.4 fused to human IgG1 Fc.

STIEEQAKYFLDKFNHEAEDLFYQSSLASWNYNTN
ITEENVQNMNNAGDKWSAFLKEQSTTAQMYPLQEI
QNLTVKLQLQALQQNGSSVLSEDKSKRLNTILNTM
STIYSTGKVCNPDNPQECLLLEPGLNEIMANSLDY
NERLWAWESWRSEVGKQLRPLYEEYVVLKNEMARA
NHYEDYGDYWRGDYEVNGVDGYDYSRGQLIEDVEH
TFEEIKPLYEHLHAYVRAKLMNAYPSYISPIGCLP
AHLLGDMWGRFWTNLYSLTVPFGQKPNIDVTDAMV
DQAWDAQRIFKEAEKFFVSVGLPNMTQGFWEYSML
TDPGNVQKAVCHPTAWDLGKGDFRILMCTKVTMDD
FLTAHHEMGHIQYDMAYAAQPFLLRNGANEGFHEA
VGEIMSLSAATPKHLKSIGLLSPDFQEDNETEINF
LLKQALTIVGTLPFTYMLEKWRWMVFKGEIPKDQW
MKKWWEMKREIVGVVEPVPHDETYCDPASLFHVSN
DYSFIRYYTRTLYQFQFQEALCQAAKHEGPLHKCD
ISNSTEAGQKLFNMLRLGKSEPWTLALENVVGAKN
MNVRPLLNYFEPLFTWLKDQNKNSFVGWSTDWSPY
ADQSIKVRISLKSALGDKAYEWNDNEMYLFRSSVA
YAMRQYFLKVKNQMILFGEEDVRVANLKPRISFNF
FVTAPKNVSDIIPRTEVEKAIRMSRSRINDAFRLN
DNSLEFLGIQPTLGSDKTHTCPPCPAPELLGGPSV
FLFPPKPKDTLMISRTPEVTCVVVDVSHEDPEVKF
NWYVDGVEVHNAKTKPREEQYNSTYRVVSVLTVLH
QDWLNGKEYKCKVSNKALPAPIEKTISKAKGQPRE
PQVYTLPPSREEMTKNQVSLTCLVKGFYPSDIAVE
WESNGQPENNYKTTPPVLDSDGSFFLYSKLTVDKS
RWQQGNVFSCSVMHEALHNHYTQKSLSLSPGK

### DETAILED DESCRIPTION

### I. Abbreviations

ACE2 angiotensin-converting enzyme 2
BLI biolayer interferometry
CoV coronavirus
COVID-19 coronavirus disease 2019
IT intratracheal
IV intravenous
MOI multiplicity of infection
RBD receptor binding domain
sACE2 soluble angiotensin-converting enzyme 2
SARS severe acute respiratory syndrome
SARS-CoV-2 SARS coronavirus 2
SEC size exclusion chromatography
sfGFP superfolder green fluorescent protein

### II. Terms and Methods

Unless otherwise noted, technical terms are used according to conventional usage. Definitions of common terms in molecular biology may be found in Benjamin Lewin, Genes X, published by Jones & Bartlett Publishers, 2009; and Meyers et al. (eds.), The Encyclopedia of CellBiology andMolecular Medicine, published by Wiley-VCH in 16 volumes, 2008; and other similar references.

As used herein, the singular forms “a,” “an,” and “the,” refer to both the singular as well as plural, unless the context clearly indicates otherwise. For example, the term “an antigen” includes single or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various embodiments, the following explanations of terms are provided:

Aerosol: A suspension of fine solid particles or liquid droplets in a gas (such as air).

Administration: To provide or give a subject an agent, such as a modified human ACE2 polypeptide, by any effective route. Exemplary routes of administration include, but are not limited to, injection (such as subcutaneous, intramuscular, intradermal, intraperitoneal, intratumoral, and intravenous), transdermal, intranasal, intratracheal and inhalation routes.

Biological sample: A sample obtained from a subject (such as a human or veterinary subject). Biological samples include, for example, fluid, cell and/or tissue samples. In some embodiments herein, the biological sample is a fluid sample. Fluid sample include, but are not limited to, serum, blood, plasma, urine, feces, saliva, cerebral spinal fluid (CSF), bronchoalveolar lavage (BAL), nasal swab, or other bodily fluid. Biological samples can also refer to cells or tissue samples, such as biopsy samples or tissue sections.

Contacting: Placement in direct physical association; includes both in solid and liquid form.

Coronavirus: A large family of positive-sense, single-stranded RNA viruses that can infect humans and non-human animals. Coronaviruses get their name from the crown-like spikes on their surface. The viral envelope is comprised of a lipid bilayer containing the viral membrane (M), envelope (E) and spike (S) proteins. Most coronaviruses cause mild to moderate upper respiratory tract illness, such as the common cold. However, three coronaviruses have emerged that can cause more serious illness and death in humans: severe acute respiratory syndrome coronavirus (SARS-CoV), SARS-CoV-2, and Middle East respiratory syndrome coronavirus (MERS-CoV). Other coronaviruses that infect humans include human coronavirus HKU1 (HKU1-CoV), human coronavirus OC43 (OC43-CoV), human coronavirus 229E (229E-CoV), human coronavirus NL63 (NL63-CoV). In some embodiments of the present disclosure, “coronavirus” includes any human coronavirus or zoonotic coronavirus that utilizes ACE2 as a cellular receptor, including known and emerging strains of coronavirus. Zoonotic coronaviruses include, but are not limited to, bat and rodent coronaviruses.

Fusion protein: A protein comprising at least a portion of two different (heterologous) proteins. In some embodiments, the fusion is comprised of a modified ACE2 polypeptide and an Fc protein, such as an Fc from human IgG1.

Heterologous: Originating from a separate genetic source or species.

Isolated: An “isolated” biological component, such as a nucleic acid or protein, has been substantially separated or purified away from other biological components in the environment (such as a cell) in which the component naturally occurs, for example other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Nebulizer: A device for converting a therapeutic agent (such as a polypeptide) in liquid form into a mist or fine spray (an aerosol) that can be inhaled into the respiratory system, such as the lungs. A nebulizer is also known as an “atomizer.”

Pharmaceutically acceptable carriers: The pharmaceutically acceptable carriers of use are conventional. Remington: The Science and Practice of Pharmacy, The University of the Sciences in Philadelphia, Editor, Lippincott, Williams, & Wilkins, Philadelphia, Pa., 21^{st} Edition (2005), describes compositions and formulations suitable for pharmaceutical delivery of the polypeptides and other compositions disclosed herein. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polypeptide, peptide and protein: Refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease. A “prophylactic” treatment is a treatment administered to a subject who does not exhibit signs of a disease or exhibits only early signs for the purpose of decreasing the risk of developing pathology. The prophylactic treatment can be pre-exposure or post-exposure.

Prophylaxis: The use of a medical treatment for preventing (or reducing the risk of developing) a disease or infection, such as a CoV infection or COVID-19. In the context of a viral infection, pre-exposure prophylaxis refers to treatment that is administered before a subject has been exposed to the virus, while post-exposure prophylaxis refers to treatment administered immediately or shortly after exposure to the virus, but before signs or symptoms of infection occur.

Purified: The term purified does not require absolute purity; rather, it is intended as a relative term. Thus, for example, a purified polypeptide preparation is one in which the polypeptide is more enriched than the polypeptide is in its natural environment, such as within a cell. In one embodiment, a preparation is purified such that the polypeptide represents at least 50% of the total peptide or protein content of the preparation. Substantial purification denotes purification from other proteins or cellular components. A substantially purified protein is at least 60%, 70%, 80%, 90%, 95% or 98% pure. Thus, in one specific, non-limiting example, a substantially purified protein is 90% free of other proteins or cellular components.

Sequence identity: The similarity between amino acid or nucleic acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide or nucleic acid molecule will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Homologs and variants of polypeptide, such as a modified human ACE2 polypeptide, are typically characterized by possession of at least about 75%, for example at least about 80%, 90%, 95%, 96%, 97%, 98% or 99% sequence identity counted over the full-length alignment with the amino acid sequence of the antibody using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Subject: Living multi-cellular vertebrate organisms, a category that includes both human and veterinary subjects, including human and non-human mammals.

Therapeutically effective amount: A quantity of a specific substance (such as a modified human ACE2 polypeptide) sufficient to achieve a desired effect in a subject being treated. For instance, this can be the amount necessary to inhibit CoV replication or reduce CoV titer in a subject. In one embodiment, a therapeutically effective amount is the amount necessary to inhibit CoV replication by at least 10%, at least 20%, at least 30%, at least 40%, at least 50%, at least 60%, at least 70%, at least 80%, or at least 90% (as compared to the absence of treatment). In another embodiment, a therapeutically effective amount is the amount necessary to reduce CoV titer in a subject by at least 10%, at least 20%, at least 30%, at least 40%, at least 50%, at least 60%, at least 70%, at least 80%, or at least 90% (as compared to the absence of treatment). The therapeutically effective amount can also be the amount necessary to reduce or eliminate one of more symptoms of CoV infection, such as the amount necessary reduce or eliminate fever, cough or shortness of breath. Similarly, in some embodiments, a prophylactically effect amount is the amount necessary to reduce the risk of becoming infected with a CoV or developing disease, such as COVID-19, by at least 20%, at least 30%, at least 40%, at least 50%, at least 60%, at least 70%, at least 80%, or at least 90% (as compared to the absence of treatment).

Vector: A nucleic acid molecule as introduced into a host cell, thereby producing a transformed host cell. A vector may include nucleic acid sequences that permit it to replicate in a host cell, such as an origin of replication. A vector may also include one or more selectable marker genes and other genetic elements known in the art. In some embodiments, the vector is a virus vector, such as a lentivirus vector.

### III Modified ACE2 Polypeptides and Methods of Use

The spike (S) glycoprotein of SARS-CoV-2 binds angiotensin-converting enzyme 2 (ACE2) on host cells. S is a trimeric class I viral fusion protein that is proteolytically processed into S1 and S2 subunits that remain noncovalently associated in a prefusion state (Walls et al., Cell. 2020 Mar 6; 181(2):281-292.e6; Hoffmann et al., Cell. 2020 Mar 4; 181(2)271-280.e8; Tortorici and Veesler, Adv Virus Res. Elsevier; 2019; 105:93-116). Upon engagement of ACE2 by a receptor binding domain (RBD) in S1 (Wong et al., J Biol Chem; 2004 Jan 30;279(5):3197-3201), conformational rearrangements occur that cause S1 shedding, cleavage of S2 by host proteases, and exposure of a fusion peptide adjacent to the S2′ proteolysis site (Tortorici and Veesler, Adv Virus Res. Elsevier; 2019; 105:93-116; Madu et al., J Virol; 2009 Aug;83(15):7411-7421; Walls et al., Proc Natl Acad Sci USA; 2017 Oct 17;114(42):11157-11162; Millet and Whittaker, Proc Natl Acad Sci USA; 2014 Oct 21;111(42):15214-15219). Favorable folding of S to a post-fusion conformation is coupled to host cell/virus membrane fusion and cytosolic release of viral RNA. Atomic contacts with the RBD are restricted to the protease domain of ACE2 (Yan et al., Science. 2020 Mar 4:eabb2762; Li et al., Science. 2005 Sep 16;309(5742):1864-1868), and soluble ACE2 (sACE2) in which the neck and transmembrane domains are removed, is sufficient for binding S and neutralizing infection (Li et al., Nature. 2003 Nov 27;426(6965):450-454; Hofmann et al., Biochem Biophys Res Commun. 2004 Jul. 9;319(4):1216-1221; Lei et al., bioRxiv. 2020 Jan 1:2020.02.01.929976; Moore et al., J Virol; 2004 Oct;78(19):10628-10635). In principle, the virus has limited potential to escape sACE2-mediated neutralization without simultaneously decreasing affinity for native ACE2 receptors, thereby attenuating virulence. Furthermore, fusion of sACE2 to the Fc region of human immunoglobulin can provide an avidity boost while recruiting immune effector functions and increasing serum stability, an especially desirable quality if intended for prophylaxis (Moore et al., J Virol; 2004 Oct;78(19):10628-10635; Liu et al., Kidney Int. 2018 July;94(1):114-125), and recombinant sACE2 has proven safe in healthy human subjects (Haschke et al., Clin Pharmacokinet. 2013 September;52(9):783-792) and patients with lung disease (Khan et al., Crit Care. 2017 Sep 7;21(1):234).

The rapid and escalating spread of SARS coronavirus 2 (SARS-CoV-2) poses an immediate public health emergency, and no approved therapeutics or vaccines are currently available. The viral spike protein S binds membrane-tethered ACE2 on host cells in the lungs to initiate molecular events that ultimately release the viral genome intracellularly. The extracellular protease domain of ACE2 inhibits cell entry of both SARS and SARS-2 coronaviruses by acting as a soluble decoy for receptor binding sites on S, and is a leading candidate for therapeutic and prophylactic development. ACE2 efficacy and manufacturability could be improved by mutations that increase affinity and expression of folded, functional protein. The present disclosure solves this challenge using deep mutagenesis and in vitro selections, whereby variants of ACE2 are identified with increased binding to the receptor binding domain of S at a cell surface. Mutations are found across the protein-protein interface and also at buried sites where they can enhance folding and presentation of the interaction epitope. In some embodiments herein, the N90-glycan on ACE2 is removed because it hinders association with S. The mutational landscape offers a blueprint for engineering high affinity ACE2 receptors to meet this unprecedented challenge. The disclosed ACE2 polypeptides are advantageous because there is very little risk of SARS-CoV-2, or any other coronavirus that binds ACE2, to develop resistance to these receptor decoys.

Described herein are ACE2 polypeptides (such as human ACE2 polypeptides) that have improved properties for binding CoV S protein. In particular, provided herein are modified ACE2 polypeptides that include a human ACE2 or a fragment thereof, such as an extracellular fragment thereof. The polypeptides include at least one amino acid substitution relative to wild-type human ACE2 (SEQ ID NO: 1).

In some embodiments, the at least one (e.g., at least one, at least two, at least three, at least four, at least five, or more) amino acid substitution is selected from any of the substitutions shown in Table 1.

In some embodiments, the at least one (e.g., at least one, at least two, at least three, at least four, at least five, or more) amino acid substitution is selected from any of the substitutions shown in Table 2.

In some embodiments, the at least one (e.g., at least one, at least two, at least three, at least four, at least five, or more) amino acid substitution is selected from any of the substitutions shown in Table 3.

In some embodiments, the at least one amino acid substitution is at residue 19, 23, 24, 25, 26, 27, 29, 30, 31, 33, 34, 35, 39, 40, 41, 42, 65, 69, 72, 75, 76, 79, 82, 89, 90, 91, 92, 324, 325, 330, 351, 386, 389, 393 and/or 518 of human ACE2 of SEQ ID NO: 1.

In some embodiments, the modified polypeptides contain only a single amino acid substitution relative to a wild-type human ACE2 (SEQ ID NO: 1), such as one amino acid substitution listed in Table 1. In other examples, the modified polypeptides include two, three, four, five or more amino acid substitutions, such as two, three, four, five or more amino acid substitutions listed in Table 1. In specific examples, the modified polypeptide includes only a single substitution at residue 19, 23, 24, 25, 26, 27, 29, 30, 31, 33, 34, 35, 39, 40, 41, 42, 65, 69, 72, 75, 76, 79, 82, 89, 90, 91, 92, 324, 325, 330, 351, 386, 389, 393 or 518 of human ACE2 of SEQ ID NO: 1. In other specific examples, the modified polypeptide includes two, three, four, five or more amino acid substitutions at residues selected from the group consisting of residues 19, 23, 24, 25, 26, 27, 29, 30, 31, 33, 34, 35, 39, 40, 41, 42, 65, 69, 72, 75, 76, 79, 82, 89, 90, 91, 92, 324, 325, 330, 351, 386, 389, 393 or 518 of human ACE2 of SEQ ID NO: 1. In other examples, the modified polypeptide includes a combination of substitutions listed in Table 4.

In some embodiments, the modified polypeptides are full-length human ACE2 polypeptides. In some examples, the amino acid sequence of the polypeptide is at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to SEQ ID NO: 1 and includes at least one amino acid substitution disclosed herein.

In other embodiments, the modified polypeptides consist of an extracellular fragment of human ACE2. For example, the modified polypeptide can consist of the complete extracellular protease domain of human ACE2, for example amino acid residues 19-615 of SEQ ID NO: 1, or the modified polypeptides can consist of a portion of the extracellular domain, such as about 50 amino acids, about 75 amino acids, about 100 amino acids, about 150 amino acids, about 200 amino acids, about 250 amino acids, about 300 amino acids, about 350 amino acids, about 400 amino acids, about 450 amino acids, about 500 amino acids, about 550 amino acids or about 590 amino acids of the extracellular domain. In some examples, the amino acid sequence of the extracellular fragment is at least 90%, at least 91%, at least 92%, at least 93%, at least 94%, at least 95% at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to residues 19 to 615 of SEQ ID NO: 1 and includes at least one amino acid substitution disclosed herein.

In some embodiments, the modified polypeptides consist of a fragment of human ACE2. In some examples, the modified polypeptides are about 50 amino acids, about 75 amino acids, about 100 amino acids, about 150 amino acids, about 200 amino acids, about 250 amino acids, about 300 amino acids, about 350 amino acids, about 400 amino acids, about 450 amino acids, about 500 amino acids, about 550 amino acids, about 590 amino acids, about 596 amino acids, about 600 amino acids, about 650 amino acids, about 700 amino acids, about 714 amino acids, about 722 amino acids, about 732 amino acids, about 740 amino acids, about 750 amino acids, or about 800 amino acids of SEQ ID NO: 1 and include at least one amino acid substitution disclosed herein. In particular non-limiting examples, the amino acid sequence of the polypeptide is at least 90%, at least 91%, at least 92%, at least 93%, at least 94%, at least 95% at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to a fragment of human ACE2, such as residues 1-732, 19-732 or 19-740 of SEQ ID NO: 1, and includes at least one amino acid substitution disclosed herein. In specific examples, the modified polypeptide consists of amino acid residues 1-732, 19-732 or 19-740 of SEQ ID NO: 1 and includes at least one amino acid substitution disclosed herein.

In some examples, the modified polypeptide comprises: T27Y, L79T, and N330Y amino acid substitutions; H34A, T92Q, Q325P, and A386L amino acid substitutions; T27Y, L79T, N330Y, and A386L amino acid substitutions; L79T, N330Y, and A386L amino acid substitutions; T27Y, N330Y, and A386L amino acid substitutions; T27Y, L79T, and A386L amino acid substitutions; A25V, T27Y, T92Q, Q325P, and A386L amino acid substitutions; H34A, L79T, N330Y, and A386L amino acid substitutions; A25V, T92Q, and A386L amino acid substitutions; or T27Y, Q42L, L79T, T92Q, Q325P, N330Y, and A386L amino acid substitutions, wherein the amino acid substitutions are with reference to SEQ ID NO: 1.

Dimers of the modified polypeptides disclosed herein are also provided. In some embodiments, the dimeric polypeptide includes residues 1-732 or 19-732 of SEQ ID NO: 1, and at least one amino acid substitution disclosed herein, such as one, two, three, four or five amino acid substitutions. In some examples, the dimer is a dimer of the sACE2v.2.4 variant having the amino acid sequence of SEQ ID NO: 10.

Also provided are fusion proteins that include a modified ACE2 polypeptide disclosed herein and a heterologous polypeptide. In some embodiments, the heterologous polypeptide is an Fc protein, such as a human Fc protein, for example the Fc from human IgG1. In specific non-limiting examples, the fusion protein comprises or consists of the amino acid sequence of SEQ ID NO: 11. In other embodiments, the heterologous polypeptide is a protein that can be used as a diagnostic/detection reagent, such as a fluorescent protein (for example, GFP) or an enzyme (for example, alkaline phosphatase, HRP or luciferase). In some embodiments, the heterologous polypeptide is an antibody or antigen-binding protein for avid binding to a second CoV antigen. In some embodiments, the heterologous polypeptide is an antibody or antigen-binding protein for tethering to cells or cellular surroundings (for example, to recruit immune cells). In some embodiments, the heterologous polypeptide is a cytokine, ligand or receptor for evoking a biological response. In some embodiments, the heterologous polypeptide is a protein that increases the serum half-life (for example, antibody Fc or serum albumin).

Compositions that include a modified ACE2 polypeptide or fusion protein thereof and a pharmaceutically acceptable carrier are also provided. In some embodiments, the modified ACE2 polypeptide or fusion protein is formulated for intratracheal or inhalation administration. Intratracheal or inhalation preparations can be liquid (e.g., solutions or suspensions) and include mists, sprays, aerosols and the like. In specific examples, the composition is formulated for administration using a nebulizer. In other embodiments, the modified ACE2 polypeptide or fusion protein is formulated for intravenous administration.

Further provided is an in vitro method of inhibiting CoV replication by contacting the CoV with a modified ACE2 polypeptide or fusion protein disclosed herein. In some examples, the CoV-infected cells (such as cultured cell lines or primary cells) are contacted with the modified ACE2 polypeptide, such as to test the effect of the modified polypeptide on CoV replication.

Methods of inhibiting CoV replication and/or spread in a subject are also provided. In some embodiments, the method includes administering to the subject a therapeutically effective amount of a modified ACE2 polypeptide, fusion protein or composition disclosed herein. Also provided is a method of treating a CoV infection (e.g. COVID-19 or SARS) in a subject, comprising administering to the subject a therapeutically effective amount of a modified ACE2 polypeptide, fusion protein or composition disclosed herein. In some examples, the subject is elderly or has an underlying medical condition (such as heart disease, lung disease, obesity, or diabetes). In some examples, the subject has COVID-19. In some examples, the subject is a healthcare worker. In some examples, the modified ACE polypeptide is administered intravenously. In other examples, the modified ACE polypeptide is administered intratracheally (IT) or via inhalation (such as by using a nebulizer). In specific non-limiting examples, the modified ACE2 polypeptide, fusion protein or composition is administered via at least two routes, such as IV and IT, or IV and inhalation. Other routes of administration to the lungs or respiratory tract include bronchial, intranasal, or other inhalatory routes, such as direct instillation in the nasotracheal or endotracheal tubes in an intubated patient. In specific non-limiting examples, the amino acid sequence of the modified ACE2 polypeptide comprises of consists of SEQ ID NO: 10 or the amino acid sequence of the fusion protein comprises or consists of SEQ ID NO: 11.

Also provided is a method of prophylactically treating (e.g. preventing) CoV infection in a subject, comprising administering to the subject a prophylactically effective amount of a modified ACE2 polypeptide, fusion protein or composition disclosed herein. Prophylactic treatment includes both pre-exposure prophylaxis and post-exposure prophylaxis. In some examples, the subject is elderly or has an underlying medical condition. In some examples, the underlying condition is cardiac disease, lung disease, obesity, or diabetes. In some examples, the subject has been exposed to patients with COVID-19. In some examples, the subject is a healthcare worker. In some examples, the modified ACE polypeptide is administered intravenously. In other examples, the modified ACE polypeptide is administered intratracheally or via inhalation (such as by using a nebulizer). Other routes of administration to the lungs or respiratory tract include bronchial, intranasal, or other inhalatory routes, such as direct instillation in the nasotracheal or endotracheal tubes in an intubated patient. In specific non-limiting examples, the amino acid sequence of the modified ACE2 polypeptide comprises of consists of SEQ ID NO: 10 or the amino acid sequence of the fusion protein comprises or consists of SEQ ID NO: 11.

In some examples of the prophylactic treatment, the treatment comprises pre-exposure prophylaxis. For example, a subject exposed to a high-risk environment, such as a health care worker or essential worker, can be administered a modified ACE polypeptide, fusion protein or composition thereof to reduce their risk of SARS-CoV-2 infection and/or development of COVID-19. In particular non-limiting examples, the pre-exposure prophylactic treatment comprises administration of the polypeptide, fusion protein or composition intratracheally or by inhalation (such as by using a nebulizer).

In some examples of the prophylactic treatment, the treatment comprises post-exposure prophylaxis. In this type of method, the subject is administered the modified ACE polypeptide, fusion protein or composition thereof immediately or shorter after exposure to SARS-CoV-2, such as within 2 hours, 4 hours, 8 hours, 12 hours, 16 hours, 20 hours or 24 hours. In particular non-limiting examples, the post-exposure prophylactic treatment comprises administration of the polypeptide, fusion protein or composition intratracheally or by inhalation (such as by using a nebulizer).

Also provided are nucleic acid molecules and vectors that encode a modified ACE2 polypeptide or fusion protein disclosed herein. In some examples, the nucleic acid molecules and vectors have different codon usage or may be codon optimized for expression in specific cell types, such as mammalian cells. In some examples, the nucleic acid molecules and vectors carry natural human polymorphisms.

Further provided are compositions that include a nucleic acid molecule or vector disclosed herein and a pharmaceutically acceptable carrier.

Methods of inhibiting CoV replication and/or spread in a subject by administering a therapeutically effective amount (or a prophylactically effective amount for pre- or post-exposure prophylactic methods) of a nucleic acid molecule, vector or composition disclosed herein are further provided. Further provided are methods of treating a CoV infection in a subject, comprising administering to the subject a therapeutically effective amount of a nucleic acid molecule, vector or composition disclosed herein. In some examples, the nucleic acid or vector is administered intravenously. In other examples, the nucleic acid or vector is administered intratracheally or via inhalation (such as by using a nebulizer). In specific non-limiting embodiments, the nucleic acid or vector is administered using at least two routes, such as IV and IT, or IV and inhalation. Other routes of administration to the lungs or respiratory tract include bronchial, intranasal, or other inhalatory routes, such as direct instillation in the nasotracheal or endotracheal tubes in an intubated patient. In some examples, the subject is elderly or has an underlying medical condition (such as heart disease, lung disease, obesity, or diabetes). In some examples, the subject has COVID-19. In some examples, the subject is a healthcare worker.

In some embodiments, the subject is administered one or more doses of a modified ACE2 polypeptide, fusion protein, nucleic acid, or composition disclosed herein. For example, the subject may be administered one or more, two or more, three or more, four or more, or five or more doses, such as twice daily, once daily, every other day, twice per week, once per week, or monthly. One of ordinary skill in the art can select an appropriate number of doses and timing of administration based on factors such as the subject being treated, condition of the subject, and underlying conditions.

Also provided herein are methods of detecting a CoV in a biological sample. In some embodiments, the method includes contacting the biological sample with a modified polypeptide or fusion protein disclosed herein; and detecting binding of the modified polypeptide or fusion protein to the biological sample. In some examples, the biological sample is a blood, saliva, sputum, nasal swab or bronchoalveolar lavage sample.

In some embodiments of the methods disclosed herein, the coronavirus is any human or animal coronavirus that utilizes ACE2 as an entry receptor, including emerging coronavirus strains. In some examples, the coronavirus is a human coronavirus. In specific examples, the human coronavirus is SARS-CoV, SARS-CoV-2, MERS-CoV, human coronavirus HKU1 (HKU1-CoV), human coronavirus OC43 (OC43-CoV), human coronavirus 229E (229E-CoV), or human coronavirus NL63 (NL63-CoV). In other examples, the coronavirus is a zoonotic coronavirus, such as a zoonotic coronavirus that has the potential to cross over to infect humans. In specific examples, the coronavirus is a bat coronavirus or a rodent coronavirus. In specific non-limiting examples, the bat coronavirus is LYRa11, Rs4231, Rs7327, Rs4084 or RsSHC014.

Further provided are kits that include a modified polypeptide or fusion protein disclosed herein bound to a solid support.

The following examples are provided to illustrate certain particular features and/or embodiments. These examples should not be construed to limit the disclosure to the particular features or embodiments described.

### Example 1

Since human ACE2 has not evolved to recognize SARS-CoV-2 S, it was hypothesized that mutations may be found that increase affinity for therapeutic and diagnostic applications. The coding sequence of full length ACE2 with an N-terminal c-myc epitope tag was diversified by introduction of degenerate codons to create a library containing all possible single amino acid substitutions at 117 sites spanning the entire interface with S and lining the substrate-binding cavity. S binding is independent of ACE2 catalytic activity (Moore et al., J Virol; 2004 Oct;78(19):10628-10635) and occurs on the outer surface of ACE2 (Yan et al., Science. 2020 Mar 4:eabb2762; Li et al., Science. 2005 Sep 16;309(5742):1864-1868), whereas angiotensin substrates bind within a deep cleft that houses the active site (Towler et al., J Biol Chem; 2004 Apr 23;279(17):17996-18007). Substitutions within the substrate-binding cleft of ACE2 therefore act as controls that are anticipated to have minimal impact on S interactions, yet may be useful for engineering out substrate affinity to enhance in vivo safety. However, the benefits of catalytically inactive sACE2 for treating COVID-19 have been questioned (Kruse, F1000Res; 2020;9(72):72).

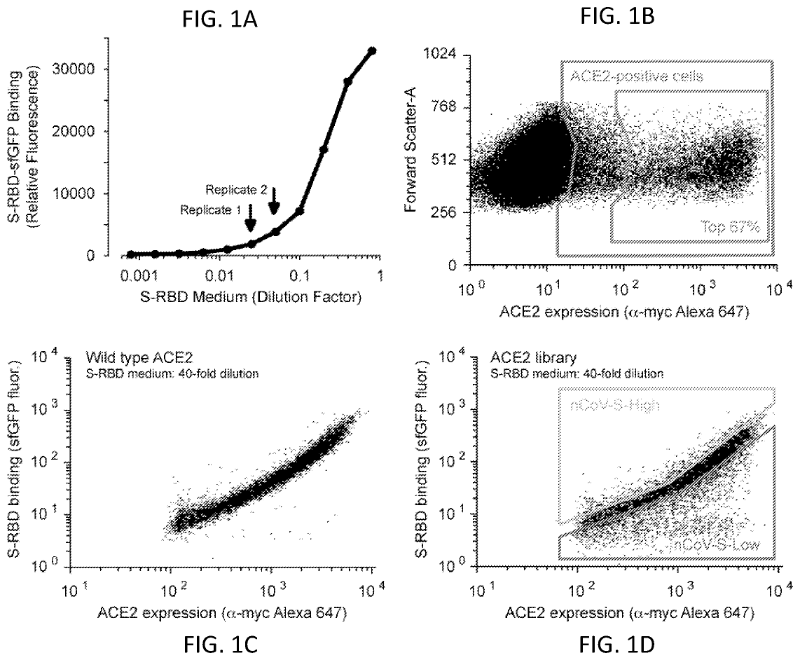

The ACE2 library was transiently expressed in human Expi293F cells under conditions that typically yield no more than one coding variant per cell, providing a tight link between genotype and phenotype (Heredia et al., J Immunol; 2018 Apr 20;200(11):jii800343-3839; Park et al., J Biol Chem; 2019; 294(13):4759-4774). Cells were then incubated with a subsaturating dilution of medium containing the RBD (a.a. 333-529 of SEQ ID NO: 2) of SARS-CoV-2 fused C-terminally to superfolder GFP (sfGFP: (Pedelacq et al., Nat Biotechnol. 2006 January;24(1):79-88)) (FIG. 1A). Levels of bound S-RBD-sfGFP correlate with surface expression levels of myc-tagged ACE2 measured by dual color flow cytometry. Compared to cells expressing wild type ACE2 (FIG. 1C), many variants in the ACE2 library failed to bind S-RBD, while there appeared to be a smaller number of ACE2 variants with higher binding signals (FIG. 1D). Cells expressing ACE2 variants with high or low binding to S-RBD were collected by fluorescence-activated cell sorting (FACS), referred to as “nCoV-S-High” and “nCoV-S-Low” sorted populations, respectively. During FACS, fluorescence signal for bound S-RBD-sfGFP continuously declined, requiring the collection gates to be regularly updated to ‘chase’ the relevant populations. This is consistent with S-RBD dissociating over hours during the experiment. Reported affinities of S-RBD for ACE2 range from 1 to 15 nM (Walls et al., Cell. 2020 Mar 6; 181(2):281-292.e6; Wrapp et al., Science. 2020 Feb 19:eabb2507).

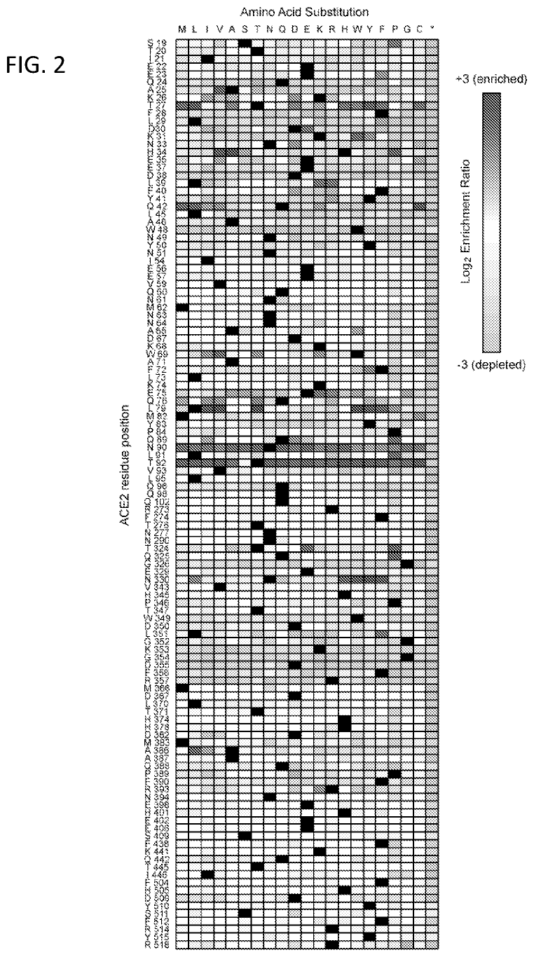

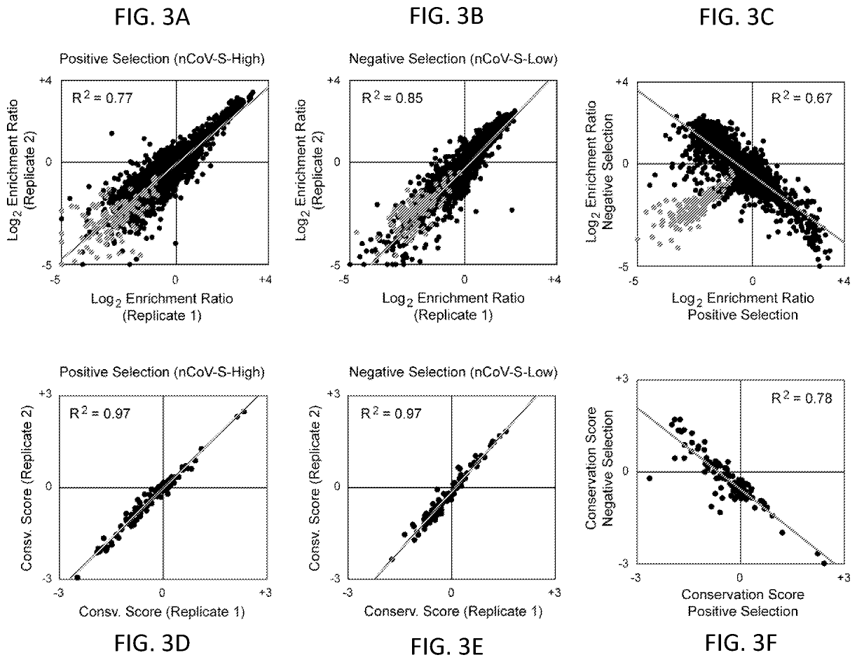

Transcripts in the sorted populations were deep sequenced, and frequencies of variants were compared to the naive plasmid library to calculate the enrichment or depletion of all 2,340 coding mutations in the library (FIG. 2). This approach of tracking an in vitro selection or evolution by deep sequencing is known as deep mutagenesis (Fowler and Fields, Nat Methods. 2014 August; 11(8):801-807). Enrichment ratios (FIGS. 3A and 3B) and residue conservation scores (FIGS. 3D and 3E) closely agree between two independent sort experiments, giving confidence in the data. For the most part, enrichment ratios (FIG. 3C) and conservation scores (FIG. 3F) in the nCoV-S-High sorts were anticorrelated with the nCoV-S-Low sorts, with the exception of nonsense mutations which were appropriately depleted from both gates. This indicated that most, but not all, nonsynonymous mutations in ACE2 did not eliminate surface expression. The library was biased towards solvent-exposed residues and has few substitutions of buried hydrophobics that might have bigger effects on plasma membrane trafficking (Park et al., J Biol Chem; 2019; 294(13):4759-4774).

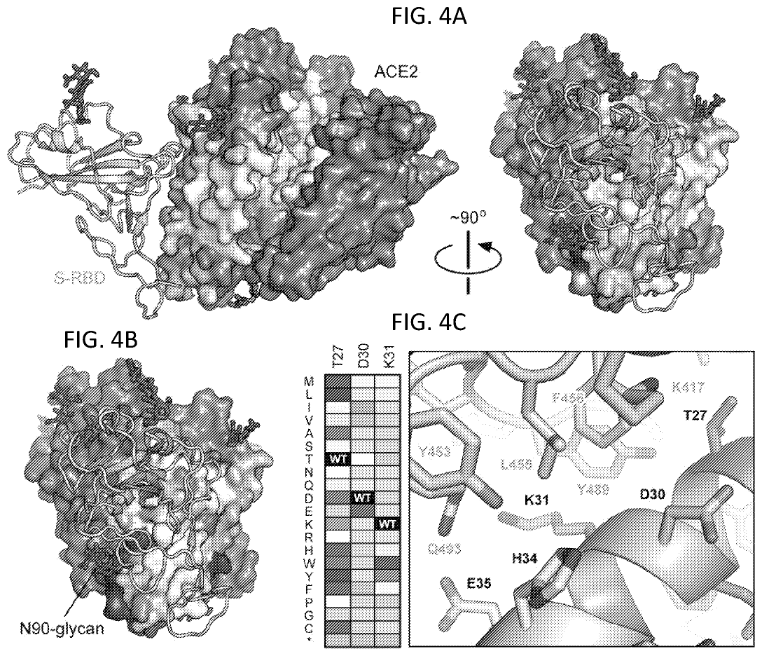

Mapping the experimental conservation scores from the nCoV-S-High sorts to the structure of S-RBD-bound ACE2 (Yan et al., Science. 2020 Mar 4:eabb2762) showed that residues buried in the interface tend to be conserved, whereas residues at the interface periphery or in the substrate-binding cleft were mutationally tolerant (FIG. 4A). The region of ACE2 surrounding the C-terminal end of the ACE2 al helix and β3-β4 strands has a weak tolerance of polar residues, while amino acids at the N-terminal end of al and the C-terminal end of α2 prefer hydrophobics (FIG. 4B), likely in part to preserve hydrophobic packing between α1-α2. These discrete patches contact the globular RBD fold and a long protruding loop of the RBD, respectively.

Two ACE2 residues, N90 and T92 that together form a consensus N-glycosylation motif, are notable hot spots for enriched mutations (FIGS. 2 and 4A). Indeed, all substitutions of N90 and T92, with the exception of T92S which maintains the N-glycan, are highly favorable for S-RBD binding, and the N90-glycan is thus predicted to partially hinder S/ACE2 interaction.

Mining the data identified many ACE2 mutations that were enriched for S-RBD binding. For instance, there were 122 mutations to 35 positions in the library that had log 2 enrichment ratios>1.5 in the nCoV-S-High sort. Table 1 lists these mutations. Table 2 lists mutations with log 2 enrichment ratios>2.0. Table 3 list mutations with log 2 enrichment ratios>2.5.

At least a dozen ACE2 mutations at the structurally characterized interface enhance S-RBD binding, and may be useful for engineering highly specific and tight binders of SARS-CoV-2 S, especially for point-of-care diagnostics. The molecular basis for how some of these mutations enhance S-RBD binding can be rationalized from the S-RBD-bound cryo-EM structure (FIG. 4C): hydrophobic substitutions of ACE2-T27 increase hydrophobic packing with aromatic residues of S-RBD, ACE2-D30E extends an acidic side chain to reach S-RBD-K417, and aromatic substitutions of ACE2-K31 contribute to an interfacial cluster of aromatics. However, engineered ACE2 receptors with mutations at the interface may present binding epitopes that are sufficiently different from native ACE2 that virus escape mutants can emerge, or they may be strain specific and lack breadth. Instead, attention was drawn to mutations in the second shell and farther that do not directly contact the S-RBD but instead have putative structural roles. For example, proline substitutions were enriched at five library positions (S19, L91, T92, T324 and Q325) where they might entropically stabilize the first turns of helices. Proline was also enriched at H34, where it may enforce the central bulge in al. Multiple mutations were also enriched at buried positions where they will change local packing (e.g. A25V, L29F, W69V, F72Y and L351F). The selection of ACE2 variants for high binding signal therefore not only reports on affinity, but also on presentation at the membrane of folded structure recognized by SARS-CoV-2 S. The presence of enriched structural mutations in the sequence landscape is especially notable considering the ACE2 library was biased towards solvent-exposed positions.

TABLE 1
ACE2 mutations with enhanced binding signals to SARS-CoV-2 S.
Log₂ enrichment ratios in selection > 1.5

| | | | | | |
|---|---|---|---|---|---|
| S19P | H34A | E75T | N90I | T92Q | R393K |
| E23F | H34S | E75K | N90V | T92D | R518G |
| Q24T | H34P | E75R | N90A | T92E | |
| A25V | E35V | E75W | N90S | T92K | |
| K26I | E35C | E75G | N90T | T92R | |
| K26A | L39K | Q76M | N90Q | T92H | |
| K26D | L39R | Q76I | N90D | T92W | |
| T27M | F40D | Q76V | N90E | T92Y | |
| T27L | F40R | Q76T | N90K | T92F | |
| T27A | Y41R | Q76R | N90R | T92P | |
| T27D | Q42M | Q76Y | N90H | T92G | |
| T27K | Q42L | L79I | N90W | T92C | |
| T27H | Q42I | L79V | N90Y | T324E | |
| T27W | Q42V | L79T | N90F | T324P | |
| T27Y | Q42K | L79W | N90P | Q325P | |
| T27F | Q42C | L79Y | N90G | N330L | |
| T27C | A65W | L79F | N90C | N330H | |
| L29F | W69I | L79P | L91P | N330W | |
| D30I | W69V | M82C | T92M | N330Y | |
| D30E | I69T | Q89I | T92L | N330F | |
| K31W | I69K | Q89D | T92I | L351F | |
| K31Y | F72Y | Q89P | T92V | A386L | |
| N33D | E75A | N90M | T92A | A386I | |
| H34V | E75S | N90L | T92N | P389D | |

TABLE 2
ACE2 mutations with enhanced binding signals to SARS-CoV-2 S.
Log₂ enrichment ratios in selection > 2.0

| | | | | |
|---|---|---|---|---|
| S19P | L39K | L79F | N90G | T92Y |
| A25V | L39R | Q89P | N90C | T92F |
| T27M | Q42M | N90M | L91P | T92P |
| T27L | Q42L | N90L | T92M | T92G |
| T27A | Q42C | N90I | T92L | T92C |
| T27D | W69V | N90V | T92I | T324E |
| T27H | F72Y | N90A | T92V | T324P |
| T27W | E75K | N90S | T92A | Q325P |
| T27Y | E75R | N90T | T92N | N330L |
| T27F | Q76V | N90Q | T92Q | N330H |
| T27C | Q76T | N90D | T92D | N330W |
| D30E | L79I | N90E | T92E | N330Y |
| K31W | L79V | N90K | T92K | N330F |
| H34V | L79T | N90R | T92R | L351F |
| H34A | L79W | N90H | T92H | A386L |
| H34P | L79Y | N90P | T92W | |

TABLE 3
ACE2 mutations with enhanced binding signals to SARS-CoV-2 S.
Log₂ enrichment ratios in selection > 2.5

| | | | | |
|---|---|---|---|---|
| A25V | L79I | N90E | T92E | N330H |
| T27M | L79V | N90H | T92R | N330W |
| T27L | L79T | L91P | T92H | N330Y |
| T27Y | L79W | T92M | T92W | N330F |
| K31W | L79Y | T92L | T92Y | A386L |
| H34V | L79F | T92I | T92F | |
| H34A | N90A | T92V | T92G | |
| H34P | N90S | T92N | T92C | |
| Q42L | N90T | T92Q | T324P | |
| Q42C | N90Q | T92D | Q325P | |

### Example 2

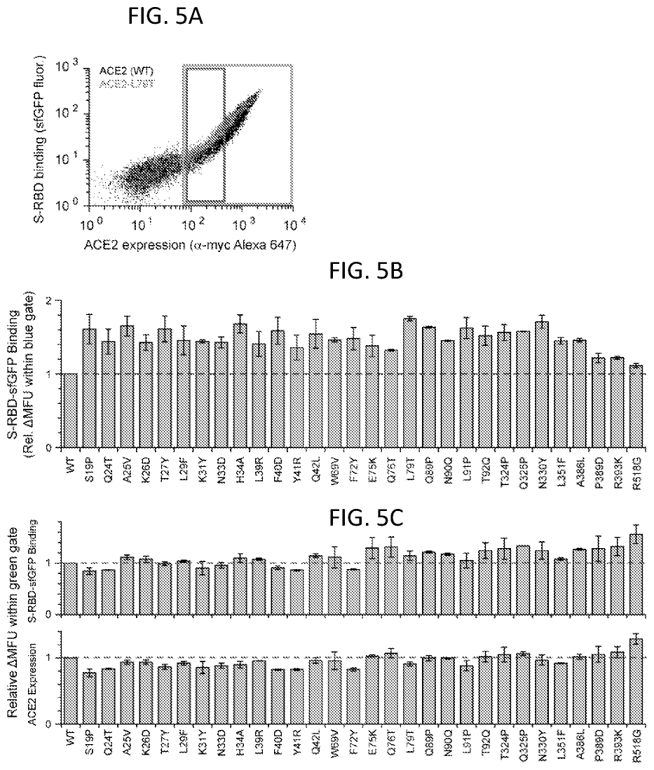

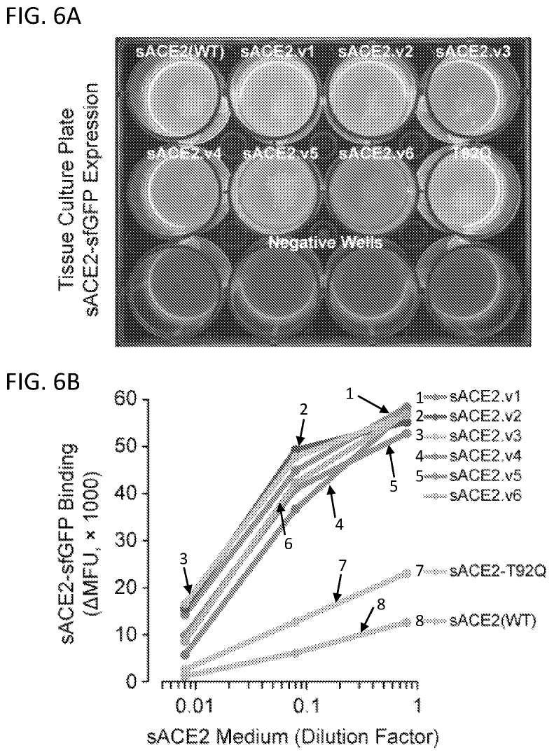

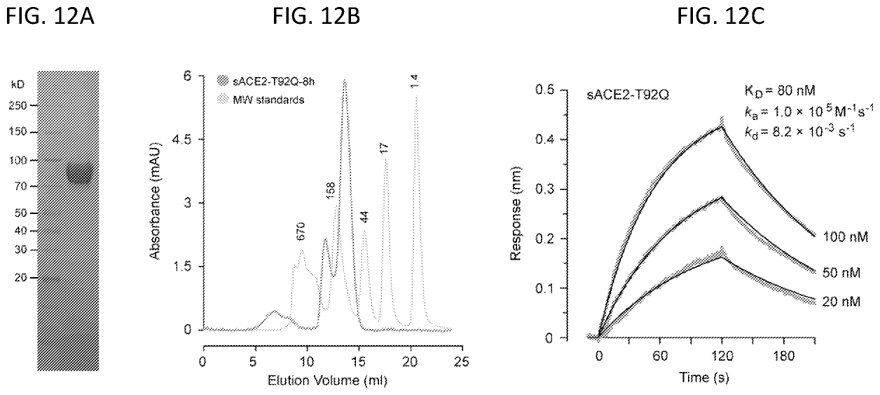

Thirty single substitutions highly enriched in the nCoV-S-High sort were validated by targeted mutagenesis (FIG. 5). Binding of RBD-sfGFP to full length ACE2 mutants increased compared to wild type, yet improvements were small and were most apparent on cells expressing low ACE2 levels (FIG. 5A). Differences in ACE2 expression between the mutants also correlated with total levels of bound RBD-sfGFP (FIG. 5C), demonstrating how one must use caution in interpreting deep mutational scan data as mutations may impact both activity and expression. To rapidly assess mutations in a format more relevant to therapeutic development, the soluble ACE2 protease domain was fused to sfGFP. Expression levels of sACE2-sfGFP were qualitatively evaluated by fluorescence of the transfected cultures (FIG. 6A), and binding of sACE2-sfGFP to full length S expressed at the plasma membrane was measured by flow cytometry (FIG. 6B). A single substitution (T92Q) that eliminates the N90 glycan gave a small increase in binding signal (FIG. 6B), which was confirmed by analysis of purified protein (FIG. 12). Focusing on the most highly enriched substitutions in the selection for S binding that were also spatially segregated to minimize negative epistasis (Heredia et al., J Virol 93(11):e00219-19, 2019), combinations of mutations in sACE2 gave large increases in S binding (Table 4 and FIG. 6B). While this assay only provides relative differences, the combinatorial mutants have enhanced binding by at least an order of magnitude. Unexplored combinations of mutations may have even greater effects.

TABLE 4
Combinatorial mutants of sACE2

| Variant | Mutations |
|---|---|
| sACE2.v1 | H34A, T92Q, Q325P, A386L |
| sACE2.v2 | T27Y, L79T, N330Y, A386L |
| sACE2.v2.1 | L79T, N330Y, A386L |
| sACE2.v2.2 | T27Y, N330Y, A386L |
| sACE2.v2.3 | T27Y, L79T, A386L |
| sACE2.v2.4 | T27Y, L79T, N330Y |
| sACE2.v3 | A25V, T27Y, T92Q, Q325P, A386L |
| sACE2.v4 | H34A, L79T, N330Y, A386L |
| sACE2.v5 | A25V, T92Q, A386L |
| sACE2.v6 | T27Y, Q42L, L79T, T92Q, Q325P, N330Y, A386L |

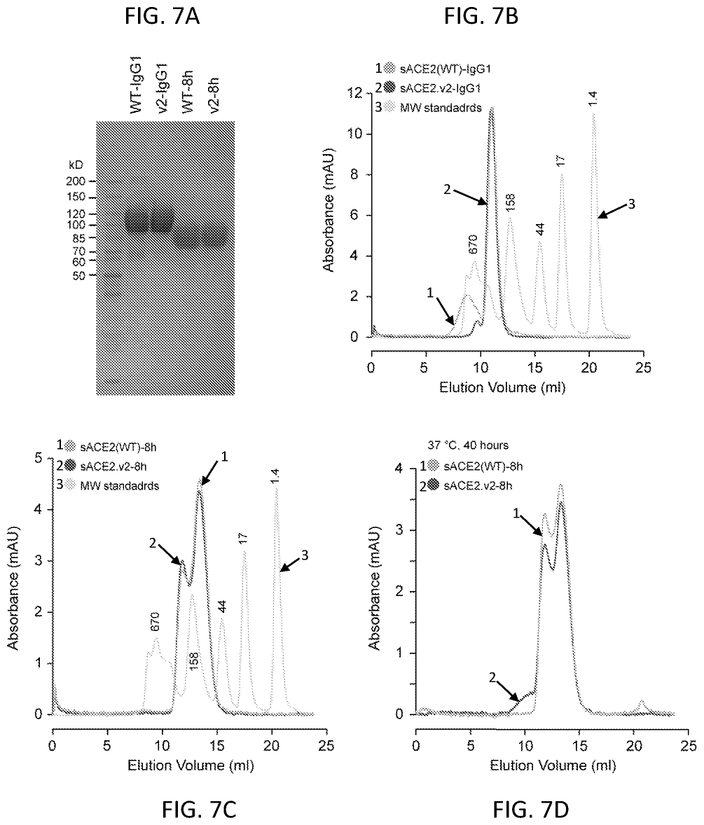

A single variant, sACE2.v2, was chosen for purification and further characterization (FIG. 7). This variant was selected because it was well expressed fused to sfGFP and maintains the N90-glycan, and will therefore present a surface that more closely matches native sACE2 to minimize immunogenicity. The yield of sACE2.v2 was lower than the wild type protein when purified as an 8his-tagged protein (20% lower) or as an IgG1-Fc fusion (60% lower), and by analytical size exclusion chromatography (SEC) a small fraction of sACE2.v2 was found to aggregate after incubation at 37° C. for 40 h (FIG. 7D). Otherwise, sACE2.v2 was indistinguishable from wild type by SEC (FIG. 7C).

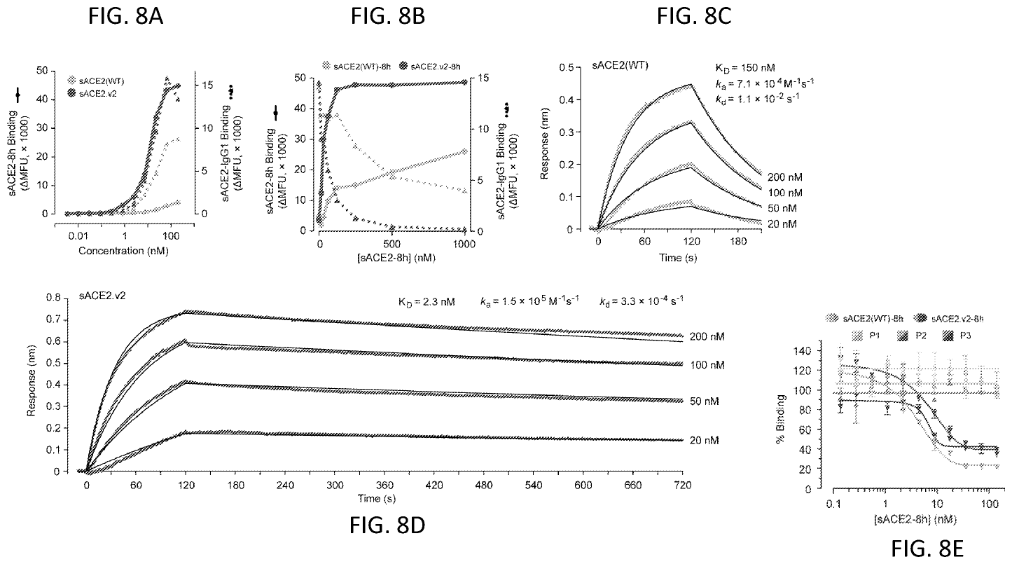

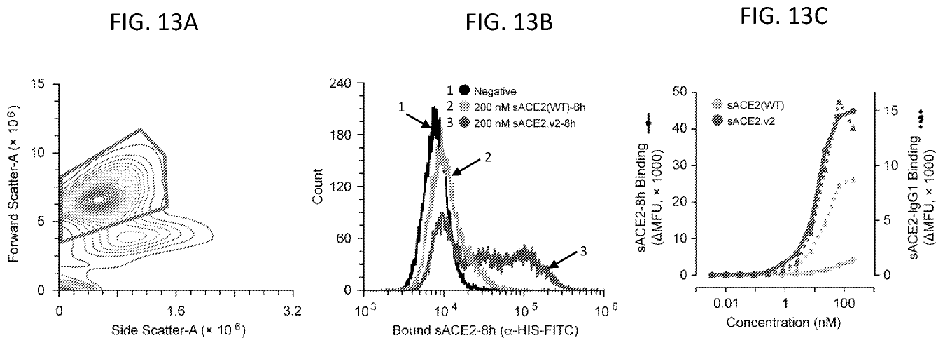

In flow cytometry experiments using purified 8his-tagged sACE2, only sACE2.v2-8h was found to bind strongly to full length S at the cell surface, suggestive that wild type sACE2 has a high off-rate that causes dissociation during sample washing (FIG. 8A and FIG. 13). Differences between wild type and the variant were less pronounced in the context of an IgG1-Fc fusion (FIG. 8A and FIG. 13), indicating that avidity masks gains in binding of the mutant, again suggestive that there are off-rate differences between wild type and variant sACE2. Soluble ACE2.v2-8h outcompetes wild type sACE2-IgG1 for binding to S-expressing cells, yet wild type sACE2-8h does not outcompete sACE2-IgG1 even at 10-fold higher concentrations (FIG. 8B). Furthermore, only engineered sACE2.v2-8h effectively competed with anti-RBD IgG in the serum of three recovered COVID-19 patients when tested by ELISA (FIG. 8E). This aligns with studies showing that while sACE2 is highly effective at inhibiting SARS-CoV-2 replication in cell lines and organoids, extremely high concentrations are required (Monteil et a., Cell DOI: 10.1016/j.cell.2020.04.004:1-28, 2020). Using biolayer interferometry (BLI), sACE2.v2 was found to have 65-fold tighter affinity than the wild type protein for immobilized RBD, almost entirely due to a slower off-rate (Table 5 and FIGS. 8C and 8D).

TABLE 5
Summary of BLI kinetics data

| Soluble Analyte | Immobilized Ligand | kₐ (M⁻¹ s⁻¹) | k_{d} (s⁻¹) | K_{D} | |
|---|---|---|---|---|---|
| sACE2(WT)-8h | RBD-IgG1 | 7.1/8.1 × 10⁴ | 1.1/1.1 × 10⁻² | 140/150 | nM |
| sACE2-T92Q-8h | RBD-IgG1 | 1.0 × 10⁵ | 8.2 × 10⁻³ | 80 | nM |
| sACE2.v2-8h | RBD-IgG1 | 1.5 × 10⁵ | 3.3 × 10⁻⁴ | 2.3 | nM |
| sACE2.v2.2-8h | RBD-IgG1 | 1.3 × 10⁵ | 8.3 × 10⁻⁴ | 6.2 | nM |
| sACE2.v2.4-8h | RBD-IgG1 | 1.4 × 10⁵ | 5.4 × 10⁻⁴ | 3.8 | nM |
| sACE2₂(WT)-8h | RBD-IgG1 | 9.5 × 10⁴ | ND | ND | |
| sACE2₂.v2.4-8h | RBD-IgG1 | 1.5 × 10⁵ | ND | ND | |
| sACE2₂.v2.4 (CHO-S) | RBD-IgG1 | 2.0 × 10⁵ | ND | ND | |
| RBD-8h | sACE2₂(WT)-IgG1 | 2.8 × 10⁵ | 6.0 × 10⁻³ | 22 | nM |
| RBD-8h | sACE2₂.v2-IgG1 | 5.8 × 10⁵ | 1.4 × 10⁻⁴ | 0.2 | nM |
| RBD-8h | sACE2₂.v2.4-IgG1 | 5.7 × 10⁵ | 3.5 × 10⁻⁴ | 0.6 | nM |

All measurements were performed using capture of IgG1(Fc)-fused proteins to an anti-human IgG Fc biosensor surface.
3-4 analyte concentrations were used for each experiment.
ND, not determined.

Across all experiments, whether ACE2 was purified as a 8his-tagged protein or used as a sfGFP-fusion in expression medium, and whether full-length S was expressed on the plasma membrane or the isolated RBD was immobilized on a biosensor surface, the characterized sACE2.v2 variant consistently showed one to two orders of magnitude tighter binding. These experiments support the key discovery from deep mutagenesis that mutations in human ACE2 exist that increase binding to S of SARS-CoV-2.

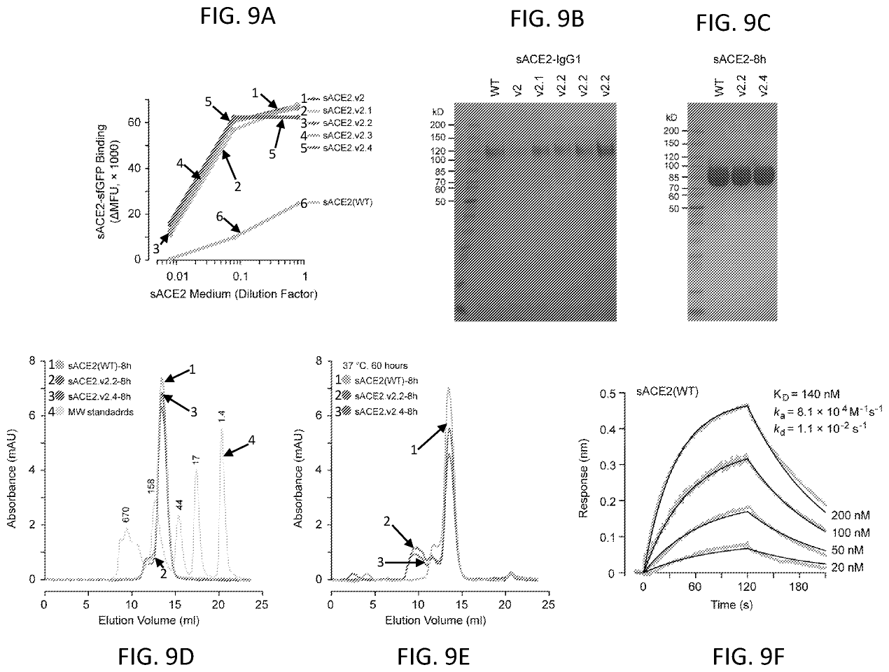

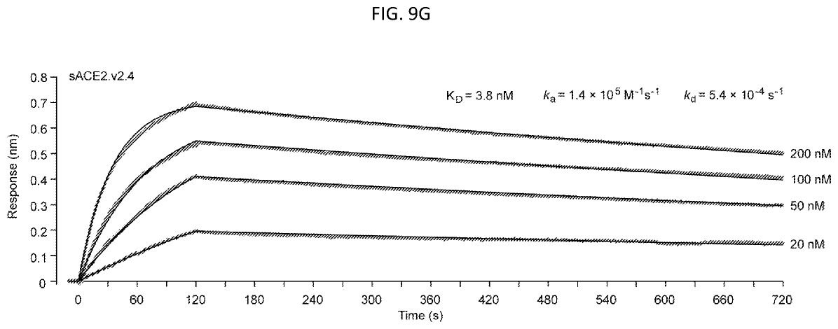

To address the decreased expression of sACE2.v2, it was hypothesized that the mutational load is too high. In second-generation designs, each of the four mutations in sACE2.v2 was reverted back to the wild type identity (Table 4) and binding to full length S at the cell surface was found to remain tight (FIG. 9A). One of the variants (sACE2.v2.4 with mutations T27Y, L79T and N330Y) was purified with even higher yields than wild type and displayed tight nanomolar binding to the RBD (FIG. 9).

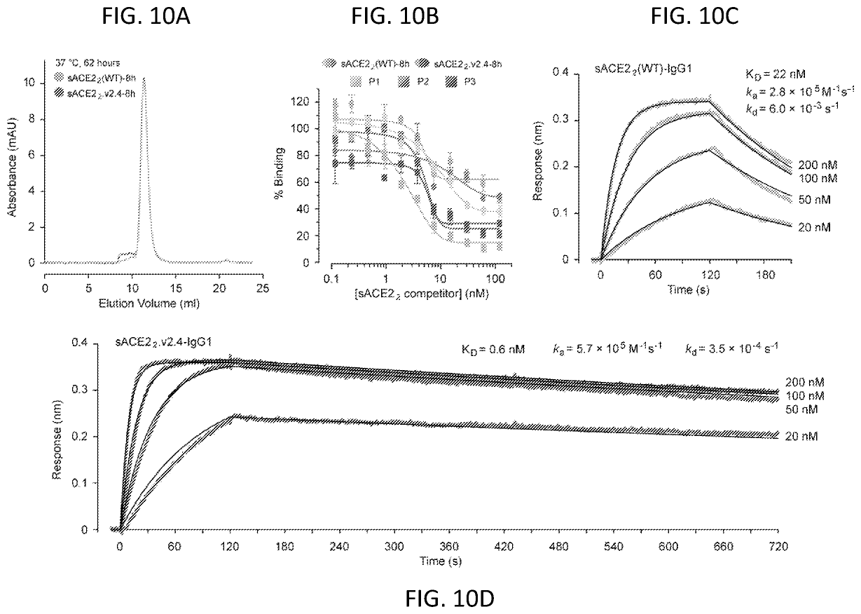

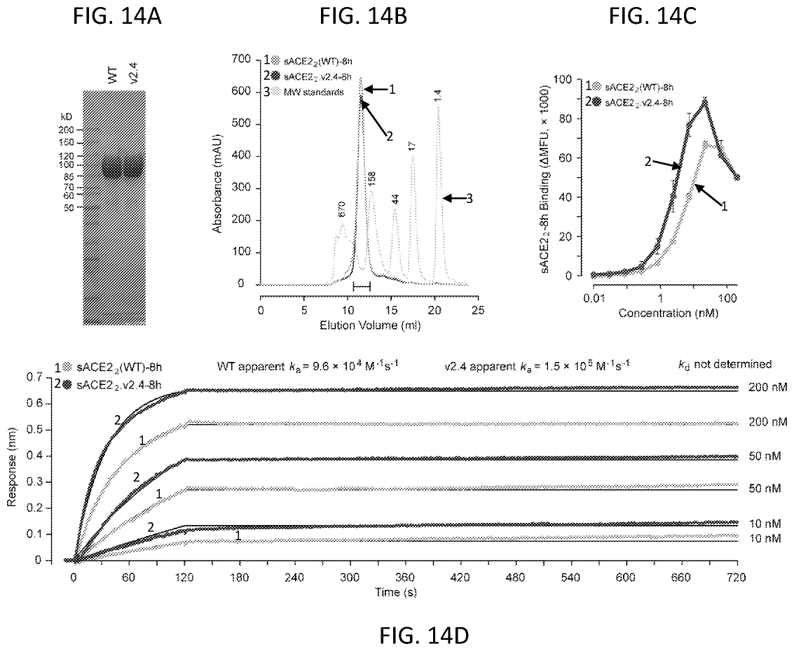

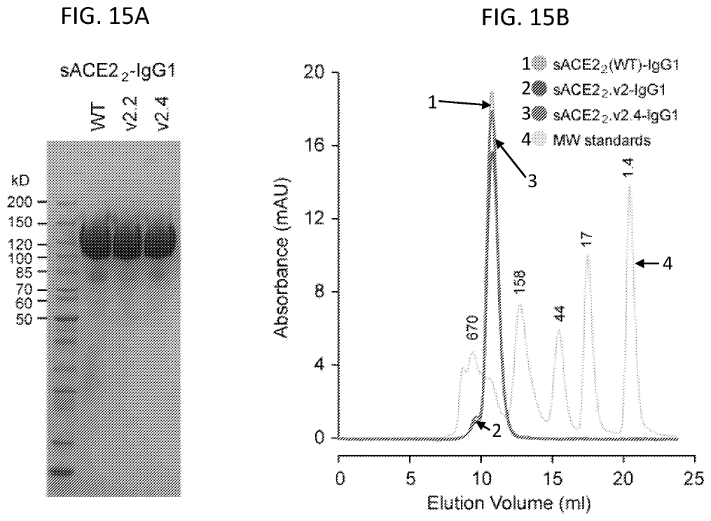

The ACE2 construct was lengthened to include the neck/dimerization domain, yielding a stable dimer (FIG. 10A) referred to here as sACE2₂, which binds with tight avidity to S on the cell surface or immobilized RBD on a biosensor (FIG. 14). Compared to the wild type, dimeric sACE2₂.v2.4 more effectively competes with IgG antibodies present in serum of recovered patients (FIG. 10B). By immobilizing sACE2₂-IgG1 (FIG. 15) to a biosensor surface and incubating with monomeric RBD-8h as the analyte, the K_{D} of RBD for wild type sACE2₂ was determined to be 22 nM (FIG. 10C) in close agreement to previous reports (Wrapp et al., Science, eabb2507, 2020; Shang et al., Nature. 382, 1199, 2020), whereas sACE2₂.v2.4 bound with 600 pM affinity (FIG. 10D). This compares favorably with recently isolated monoclonal antibodies (Pinto et al., Nature doi:10.1038/s41586-020-2349-y, 2020; Hansen et al., Science, eabd0827, 2020; Brouwer et al., Science, eabc5902, 2020; Wec et al., Science, eabc7424, 2020; Wang et al., Nat Commun. 11, 2251, 2020; Wu et al., Science. 368, 1274, 2020; Rogers et al., Science, eabc7520, 2020).

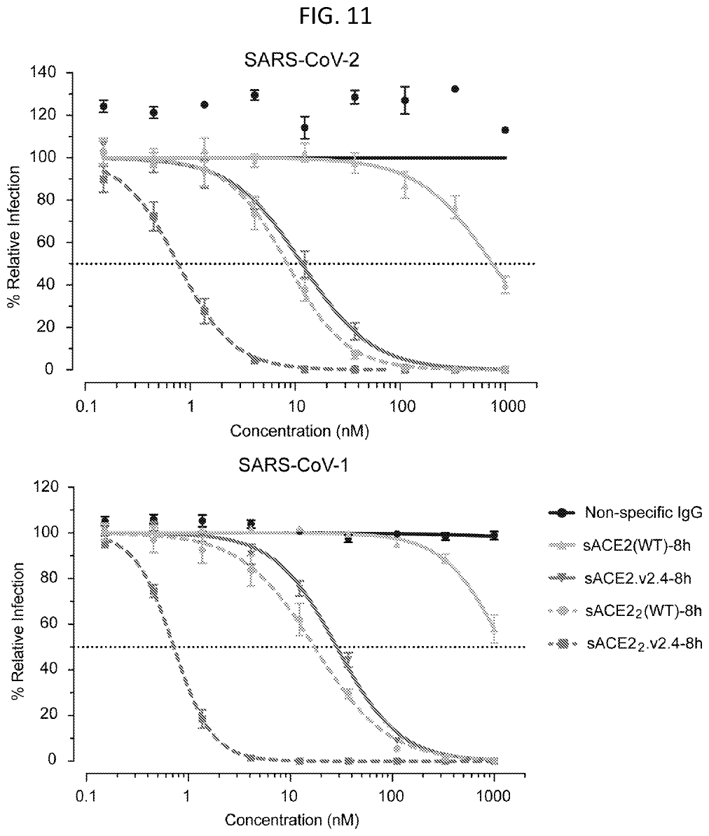

The efficacy of monomeric sACE2.v2.4 to neutralize SARS-CoV-2 infection of cultured VeroE6 cells exceeded the wild type protein by nearly two orders of magnitude (FIG. 11), consistent with the biochemical binding data. Wild type, dimeric sACE2₂ is itself two orders of magnitude more potent than monomer, indicating strong avid interactions with spike on the virion surface, and dimeric sACE2₂.v2.4 is yet again more potent with a subnanomolar IC₅₀ (FIG. 11). Dimeric sACE2₂.v2.4 also potently neutralizes SARS-CoV-1 (FIG. 11), despite no consideration of SARS-CoV-1 S structure or sequence during the engineering process, and it is possible that the decoy receptor will neutralize diverse ACE2-utilizing coronaviruses that have yet to cross over to humans.

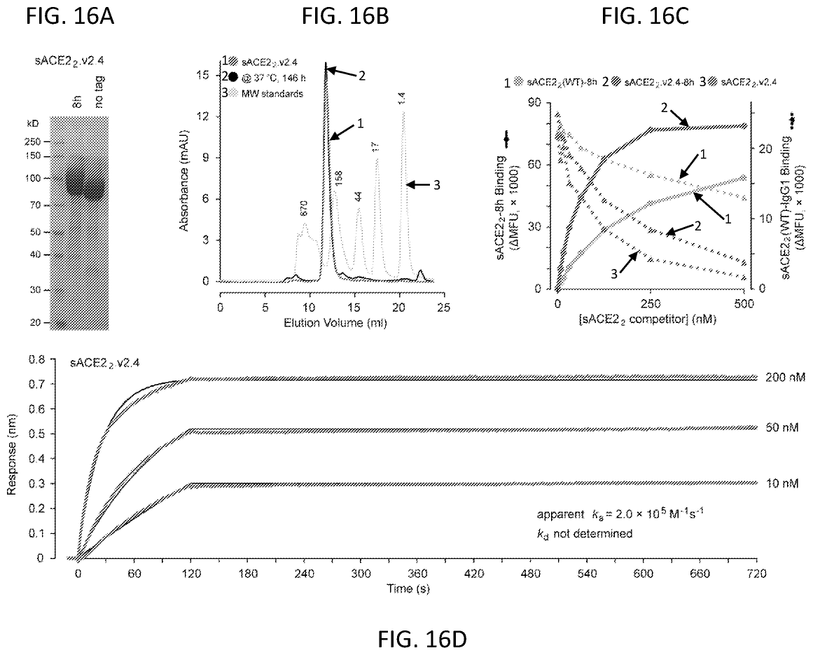

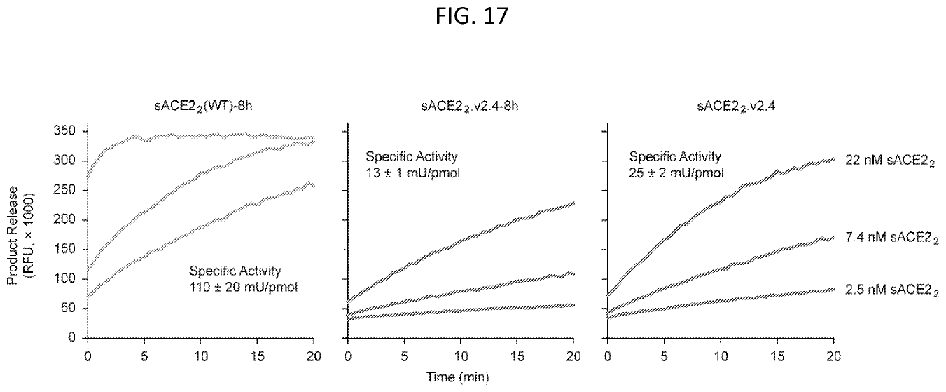

To improve safety, untagged sACE2₂.v2.4 was manufactured in ExpiCHO-S cells (FIG. 16A) and found to be stable after incubation at 37° C. for 6 days (FIG. 16B). The protein competes with wild type sACE2₂-IgG1 for cell-expressed S (FIG. 16C) and binds with tight avidity to immobilized RBD (FIG. 16D). In addition to inhibiting virus entry, recombinant sACE2 may have a second therapeutic mechanism: proteolysis of angiotensin II (a vasoconstrictive peptide hormone) to relieve symptoms of respiratory distress (Imai et al., Nature. 436, 112-116, 2005; Treml et al., Crit. Care Med. 38, 596-601, 2010). Soluble ACE22.v2.4 is found to be catalytically active, albeit with reduced activity (FIG. 17).

While deep mutagenesis of viral proteins in replicating viruses has been extensively pursued to understand escape mechanisms from drugs and antibodies, the work here shows how deep mutagenesis can be directly applicable to therapeutic design when the selection method is decoupled from virus replication and focused on host factors. With astonishing speed, the scientific community has identified multiple candidates for the treatment of COVID-19, especially monoclonal antibodies with exceptional affinity for protein S. The studies disclosed herein show how comparable affinity can be engineered into the virus' natural receptor, while also providing insights in to the molecular basis for initial virus-host interactions.

### Example 3

Materials and Methods

Plasmids. The mature polypeptide (a.a. 19-805) of human ACE2 (GenBank NM_021804.1) was cloned in to the NheI-XhoI sites of pCEP4 (Invitrogen) with a N-terminal HA leader (MKTIIALSYIFCLVFA), myc-tag, and linker (GSPGGA). Soluble ACE2 fused to superfolder GFP (Pedelacq et al., Nat. Biotechnol. 24, 79-88, 2006) was constructed by genetically joining the protease domain (a.a. 1-615) of ACE2 to sfGFP (GenBank ASL68970) via a gly/ser-rich linker (GSGGSGSGG), and pasting between the NheI-XhoI sites of pcDNA3.1(+) (Invitrogen). Equivalent sACE2 constructs were cloned with a GSG linker and 8 histidine tag or a GS linker and the Fc region of IgG1 (a.a. D221-K447), while dimeric sACE2₂ constructs encompassed a.a. 1-732 and were otherwise identical. A synthetic human codon-optimized gene fragment (Integrated DNA Technologies) for the RBD (a.a. 333-529) of SARS-CoV-2 S (GenBank YP_009724390.1) was N-terminally fused to a HA leader and C-terminally fused to either superfolder GFP, the Fc region of IgG1 or a 8 histidine tag. Assembled DNA fragments were ligated in to the NheI-XhoI sites of pcDNA3.1(+). Human codon-optimized full length S was subcloned from pUC57-2019-nCoV-S(Human) (Molecular Cloud), both untagged (a.a. 1-1273) and with a N-terminal HA leader (MKTIIALSYIFCLVFA), myc-tag and linker (GSPGGA) upstream of the mature polypeptide (a.a. 16-1273).

Tissue Culture. Expi293F cells (ThermoFisher) were cultured in Expi293 Expression Medium (ThermoFisher) at 125 rpm, 8% C02, 37° C. For production of RBD-sfGFP, RBD-IgG1, sACE2-8h and sACE2-IgG1, cells were prepared to 2×10⁶/ml. Per ml of culture, 500 ng of plasmid and 3 μg of polyethylenimine (MW 25,000; Polysciences) were mixed in 100 μl of OptiMEM (Gibco), incubated for 20 minutes at room temperature, and added to cells. Transfection Enhancers (ThermoFisher) were added 18-23 h post-transfection, and cells were cultured for 4-5 days. Cells were removed by centrifugation at 800×g for 5 minutes and medium was stored at −20° C. After thawing and immediately prior to use, remaining cell debris and precipitates were removed by centrifugation at 20,000×g for 20 minutes. Plasmids for expression of sACE2-sfGFP protein were transfected in to Expi293F cells using Expifectamine (ThermoFisher) according to the manufacturer's directions, with Transfection Enhancers added 22/2 h post-transfection, and medium supernatant harvested after 60 h.

Deep mutagenesis. 117 residues within the protease domain of ACE2 were diversified by overlap extension PCR (Procko et al., J. Mol. Biol. 425, 3563-3575, 2013) using primers with degenerate NNK codons. The plasmid library was transfected in to Expi293F cells using Expifectamine under conditions previously shown to typically give no more than a single coding variant per cell (Heredia et al., J. Immunol. 200, ji1800343-3839, 2018; Park et al., J Biol Chem 294, 4759-4774, 2019); 1 ng coding plasmid was diluted with 1,500 ng pCEP4-ΔCMV carrier plasmid per ml of cell culture at 2×10⁶/ml, and the medium was replaced 2 h post-transfection. The cells were collected after 24 h, washed with ice-cold PBS supplemented with 0.2% bovine serum albumin (PBS-BSA), and incubated for 30 minutes on ice with a 1/20 (replicate 1) or 1/40 (replicate 2) dilution of medium containing RBD-sfGFP into PBS-BSA. Cells were co-stained with anti-myc Alexa 647 (clone 9B11, 1/250 dilution; Cell Signaling Technology). Cells were washed twice with PBS-BSA, and sorted on a BD FACS Aria II at the Roy J. Carver Biotechnology Center. The main cell population was gated by forward/side scattering to remove debris and doublets, and DAPI was added to the sample to exclude dead cells. Of the myc-positive (Alexa 647) population, the top 67% were gated (FIG. 1B). Of these, the 15% of cells with the highest and 20% of cells with the lowest GFP fluorescence were collected (FIG. 1D) in tubes coated overnight with fetal bovine serum and containing Expi293 Expression Medium. Total RNA was extracted from the collected cells using a GeneJET RNA purification kit (Thermo Scientific), and cDNA was reverse transcribed with high fidelity Accuscript (Agilent) primed with gene-specific oligonucleotides. Diversified regions of ACE2 were PCR amplified as 5 fragments. Flanking sequences on the primers added adapters to the ends of the products for annealing to Illumina sequencing primers, unique barcoding, and for binding the flow cell. Amplicons were sequenced on an Illumina NovaSeq 6000 using a 2×250 nt paired end protocol. Data were analyzed using Enrich (Fowler et al., Bioinformatics. 27, 3430-3431, 2011), and commands are provided in the GEO deposit. Briefly, the frequencies of ACE2 variants in the transcripts of the sorted populations were compared to their frequencies in the naive plasmid library to calculate a log 2 enrichment ratio and then normalized by the same calculation for wild type. Wild type sequences were neither substantially enriched or depleted, and had log 2 enrichment ratios of −0.2 to +0.2.

Flow Cytometry Analysis of ACE2-S Binding. Expi293F cells were transfected with pcDNA3-myc-ACE2, pcDNA3-myc-S or pcDNA3-S plasmids (500 ng DNA per ml of culture at 2×10⁶/ml) using Expifectamine (ThermoFisher). Cells were analyzed by flow cytometry 24 h post-transfection. To analyze binding of RBD-sfGFP to full length myc-ACE2, cells were washed with ice-cold PBS-BSA, and incubated for 30 minutes on ice with a 1/30 dilution of medium containing RBD-sfGFP and a 1/240 dilution of anti-myc Alexa 647 (clone 9B 11, Cell Signaling Technology). Cells were washed twice with PBS-BSA and analyzed on a BD LSR II. To analyze binding of sACE2-sfGFP to full length myc-S, cells were washed with PBS-BSA, and incubated for 30 minutes on ice with a serial dilution of medium containing sACE2-sfGFP and a 1/240 dilution of anti-myc Alexa 647 (clone 9B11, Cell Signaling Technology). Cells were washed twice with PBS-BSA and analyzed on a BD Accuri C6, with the entire Alexa 647-positive population gated for analysis. To measure binding of sACE2-IgG1 or sACE2-8h, myc-S or S transfected cells were washed with PBS-BSA and incubated for 30 minutes with the indicated concentrations of purified sACE2 in PBS-BSA. Cells were washed twice, incubated with secondary antibody ( 1/100 dilution of chicken anti-HIS-FITC polyclonal from Immunology Consultants Laboratory; or 1/250 anti-human IgG-APC clone HP6017 from BioLegend) for 30 minutes on ice, washed twice again, and fluorescence of the total population after gating by FSC-SSC to exclude debris was measured on a BD Accuri C6. Data were processed with FCS Express (De Novo Software) or BD Accuri C6 Software.

Purification of IgG1-Fc fused proteins. Cleared expression medium was incubated with KANEKA KanCapA 3G Affinity sorbent (Pall; equilibrated in PBS) for 90 minutes at 4° C. The resin was collected on a chromatography column, washed with 12 column volumes (CV) PBS, and protein eluted with 5 CV 60 mM Acetate pH 3.7. The eluate was immediately neutralized with 1 CV of 1 M Tris pH 9.0, and concentrated with a 100 kD MWCO centrifugal device (Sartorius). Protein was separated on a Superdex 200 Increase 10/300 GL column (GE Healthcare Life Sciences) with PBS as the running buffer. Peak fractions were pooled, concentrated to ˜10 mg/ml with excellent solubility, and stored at −80° C. after snap freezing in liquid nitrogen. Protein concentrations were determined by absorbance at 280 nm using calculated extinction coefficients for monomeric, mature polypeptide sequences.

Purification of 8his-tagged proteins. HisPur Ni-NTA resin (Thermo Scientific) equilibrated in PBS was incubated with cleared expression medium for 90 minutes at 4° C. The resin was collected on a chromatography column, washed with 12 column volumes (CV) PBS, and protein eluted with a step elution of PBS supplemented with 20 mM, 50 mM and 250 mM imidazole pH 8 (6 CV of each fraction). The 50 mM and 250 mM imidazole fractions were concentrated with a 30 kD MWCO centrifugal device (MilliporeSigma). Protein was separated on a Superdex 200 Increase 10/300 GL column (GE Healthcare Life Sciences) with PBS as the running buffer. Peak fractions were pooled, concentrated to −5 mg/ml with excellent solubility, and stored at −80° C. after snap freezing in liquid nitrogen.

Other proteins. Untagged sACE2₂.v2.4 expressed in ExpiCHO-S cells (ThermoFisher) was manufactured and provided by Orthogonal Biologics, Inc.

Analytical size exclusion chromatography (SEC). Proteins (200 μl at 2 μM) were separated on a Superdex 200 Increase 10/300 GL column (GE Healthcare Life Sciences) equilibrated in PBS. MW standards were from Bio-Rad.

Biolayer Interferometry. Hydrated anti-human IgG Fc biosensors (Molecular Devices) were dipped in expression medium containing RBD-IgG1 for 60 s. Biosensors with captured RBD were washed in assay buffer, dipped in the indicated concentrations of sACE2-8h protein, and returned to assay buffer to measure dissociation. Data were collected on a BLItz instrument and analyzed with a 1:1 binding model using BLItz Pro Data Analysis Software (Molecular Devices). The assay buffer was 10 mM HEPES pH 7.6, 150 mM NaCl, 3 mM EDTA, 0.05% polysorbate 20, 0.5% non-fat dry milk (Bio-Rad).

Reagent and data availability. Plasmids are deposited with Addgene under IDs 141183-5, 145145-78, 149268-71, 149663-8 and 154098-106. Raw and processed deep sequencing data are deposited in NCBI's Gene Expression Omnibus (GEO) with series accession no. GSE147194.

ACE2 catalytic activity assay. Activity was measured using the Fluorometric ACE2 Activity Assay Kit (BioVision) with protein diluted in assay buffer to 22, 7.4 and 2.5 nM final concentration. Specific activity is reported as pmol MCA produced per min (mU) per pmol of enzyme. Fluorescence was read on an Analyst HT (Molecular Devices).

ELISA. Anti-RBD IgG titers were measured in human serum samples by indirect ELISA as described in Amant et al. (Nat. Med. 5, 562, 2020). Wells of a 96-well plate were coated with 2 μg/ml RBD-8h protein at 4° C. overnight. After washing, the wells were blocked with PBS containing 3% non-fat milk at room temperature for 1 hour. Next, various dilutions of heat-inactivated serum (56° C., 1 hour) were added to blocked wells. After 2 hours at room temperature, wells were washed, followed by incubation with goat anti-human IgG-HRP (ThermoFisher) for 1 hour at room temperature. Any unbound HRP-conjugated antibody was removed by washing, and TMB substrate for HRP was added. The colorimetric reaction was developed for 10 minutes, following which 2N sulfuric acid was added to stop the reaction. Absorbance of the product was measured at 450 nm. For competition assays, dilutions of serum (equivalent to their titers: 1:5000 for P1, 1:2000 for P2 and 1:1000 for P3) were pre-mixed with various concentrations of sACE2. Serum-sACE2 mixtures were added to blocked plates and the protocol continued as described above.

Not Human Subjects Research (NHSR) determination. De-identified serum samples from recovered COVID-19 patients were provided by the University of Chicago (patients P2 and P3) and by commercial vendor (patient P1; RayBiotech). The Office for the Protection of Research Subjects at the University of Illinois determined that the use of the samples in the ELISA study did not meet the criteria for Human Subjects Research as defined in 45CFR46(d)(f) or 21CFR56.102(c)(e) and did not require IRB approval.

Virus microneutralization assay. Vero E6 cells were cultured and their infection by authentic SARS-CoV-2 were assayed as described in Wec et al. (Science, eabc7424, 2020). Briefly, soluble ACE2 proteins were serially diluted in culture medium and incubated with SARS-CoV-2 (virus isolate 2019-nCoV/USA-WA1-A12/2020; GenBank Acc. No. MT020880.1) for 1 h. The mixture was added to VeroE6 cells at a MOI of 0.2 and incubated for 24 hrs. Cells were fixed and immunostained with anti-SARS-CoV-2 nucleocapsid antibody (Sino Biological) and an Alexa Fluor 488-conjugated goat anti-rabbit secondary. Plates were imaged on an Operetta (PerkinElmer) to determine the number of infected cells and compared to virus only control wells to calculate the percent of relative infection.

### Example 4

Zoonotic coronaviruses have crossed over from animal reservoirs multiple times in the past two decades, and it is almost certain that wild animals will continue to be a source of devastating outbreaks. Unlike ubiquitous human coronaviruses responsible for common respiratory illnesses, these zoonotic coronaviruses with pandemic potential cause serious and complex diseases, in part due to their tissue tropisms driven by receptor usage. Severe Acute Respiratory Syndrome Coronaviruses 1 (SARS-CoV-1) and 2 (SARS-CoV-2) engage angiotensin-converting enzyme 2 (ACE2) for cell attachment and entry (Zhou et al., Nature. 579, 270-273, 2020; Walls et al., Cell, 2020), doi:10.1016/j.cell.2020.02.058; Wan et al., SARS. J. Virol., 2020), doi:10.1128/JVI.00127-20; Wrapp et al., Science, eabb2507, 2020; Hoffmann et al., Cell, 2020), doi:10.1016/j.cell.2020.02.052; Li et al., Nature. 426, 450-454, 2003; Letko et al., Nat Microbiol. 11, 1860, 2020). ACE2 is a protease responsible for regulating blood volume and pressure that is expressed on the surface of cells in the lung, heart and gastrointestinal tract, among other tissues (Samavati, B. D. Uhal, Front. Cell. Infect. Microbiol. 10, 752, 2020; Jiang et al., Nat Rev Cardiol. 11, 413-426, 2014). The ongoing spread of SARS-CoV-2 and the disease it causes, COVID-19, has had a crippling toll on global healthcare systems and economies, and effective treatments and vaccines are urgently needed.

As SARS-CoV-2 becomes endemic in the human population, it has the potential to mutate and undergo genetic drift. To what extent this will occur as increasing numbers of people are infected and mount counter immune responses is unknown, but already a variant in the viral spike protein S (D614G) has rapidly emerged from multiple independent events and effects S protein stability and dynamics (Zhang et al., bioRxiv, 2020.06.12.148726, 2020; Korber et al., Cell. 182, 812-827.e19, 2020). Another S variant (D839Y) became prevalent in Portugal, possibly due to a founder effect (Borges et al., medRxiv, 2020.08.10.20171884, 2020). Coronaviruses have moderate to high mutation rates (measured at 10⁻⁴ substitutions per year per site in HCoV-NL63 (Pyrc et al., J. Mol. Biol. 364, 964-973, 2006), an alphacoronavirus that also binds ACE2, albeit via a smaller interface that is only partially shared with the RBDs of SARS-associated betacoronaviruses (Wu et al., Proc. Natl. Acad. Sci. U.S.A. 106, 19970-19974, 2009)), and large changes in coronavirus genomes have frequently occurred in nature from recombination events, especially in bats where co-infection levels can be high (Su et al., Trends Microbiol. 24, 490-502, 2016; Boni et al., Nat Microbiol 5, 1408-1417, 2020). Recombination of MERS-CoVs has also been documented in camels (Sabir et al., Science. 351, 81-84, 2016). This will all have profound implications for the current pandemic's trajectory, the potential for future coronavirus pandemics, and whether drug resistance in SARS-CoV-2 becomes prevalent.

The viral spike is a vulnerable target for neutralizing monoclonal antibodies that are progressing to the clinic, yet in tissue culture escape mutations in the spike rapidly emerge to all antibodies tested (Baum et al., Science, eabd0831, 2020). Deep mutagenesis of the isolated receptor-binding domain (RBD) by yeast surface display has easily identified mutations in S that retain high expression and ACE2 affinity, yet are no longer bound by monoclonal antibodies and confer resistance (Greaney et al., bioRxiv, 2020.09.10.292078, 2020). This has motivated the development of cocktails of non-competing monoclonal antibodies (Baum et al., Science, eabd0831, 2020; Tortorici et al., Science, eabe3354, 2020), inspired by lessons learned from the treatment of HIV-1 and Ebola, to limit the possibilities for the virus to escape. This does not yet address future coronavirus spill overs from wild animals that may be antigenically distinct. Indeed, large screening efforts were required to find antibodies from recovered SARS-CoV-1 patients that cross-react with SARS-CoV-2 (Pinto et al., Nature, 2020), doi:10.1038/s41586-020-2349-y), indicating antibodies have confined capacity for interacting with variable epitopes on the spike surface, and are unlikely to be broad and pan-specific for all SARS-related viruses.

An alternative protein-based antiviral to monoclonal antibodies is to use soluble ACE2 (sACE2) as a decoy to compete for receptor-binding sites on the viral spike (Li et al., Nature. 426, 450-454, 2003; Hofmann et al., Biochem. Biophys. Res. Commun. 319, 1216-1221, 2004; Lei et al., Nat Commun. 11, 2070, 2020; Monteil et al., Cell, 2020, doi:10.1016/j.cell.2020.04.004; Chan et al., Science. 4, eabcO870, 2020). In principle, the virus has limited potential to escape sACE2-mediated neutralization without simultaneously decreasing affinity for the native ACE2 receptor, rendering the virus less virulent. Multiple groups have now engineered sACE2 to create high affinity decoys for SARS-CoV-2 that rival matured monoclonal antibodies and potently neutralize infection (Chan et al., Science. 4, eabcO870, 2020; Glasgow et al., bioRxiv, 2020.07.31.231746, 2020; Higuchi et al., bioRxiv, 2020.09.16.299891, 2020). In the study disclosed herein, deep mutagenesis was used to identify a large number of mutations in ACE2 that increase affinity for S (Chan et al., Science. 4, eabcO870, 2020). These mutations were dispersed across the interface and also at distal sites where they are predicted to enhance folding of the virus-recognized conformation. A combination of three mutations, called sACE2₂.v2.4, increases affinity 35-fold and binds SARS-CoV-2 S (K_{D} 600 μM) with affinity comparable to the best monoclonal antibodies (Chan et al., Science. 4, eabcO870, 2020). Even tighter apparent affinities are reached through avid binding to trimeric spike expressed on a membrane. Despite engineering being focused exclusively on SARS-CoV-2 affinity, sACE2₂.v2.4 potently neutralized authentic SARS-CoV-1 and −2 infection in tissue culture, suggesting it's close resemblance to the wild type receptor confers broad activity against ACE2-utilizing betacoronaviruses generally. Soluble ACE22.v2.4 is dimeric and monodisperse without aggregation, catalytically active, highly soluble, stable after storage at 37° C. for days, and well expressed at levels greater than the wild type protein. Due to its favorable combination of high activity and desirable properties for manufacture, sACE2₂.v2.4 is a genuine drug candidate for preclinical development.

Engineered, high affinity decoy receptors, while very similar to natural ACE2, nonetheless have mutations present at or near the interaction surface. There is therefore an opportunity for viral spike variants to discriminate between an engineered decoy and wild type receptors, providing a route towards resistance. It is disclosed herein that the engineered decoy sACE2₂.v2.4 binds broadly and tightly to the RBDs of diverse SARS-associated betacoronaviruses that use ACE2 for entry. Mutations were not found within the RBD, which directly contacts ACE2 and is where possible escape mutations will most likely reside, that redirect specificity towards the wild type receptor, although many mutations that favor binding of the engineered decoy were found in a competition binding assay. The results demonstrate that resistance to an engineered decoy receptor will be rare, and sACE2₂.v2.4 targets common attributes for affinity to S in SARS-associated viruses.

### Results

An Engineered Decoy Receptor Broadly Binds RBDs from SARS-Associated CoVs with Tight Affinity

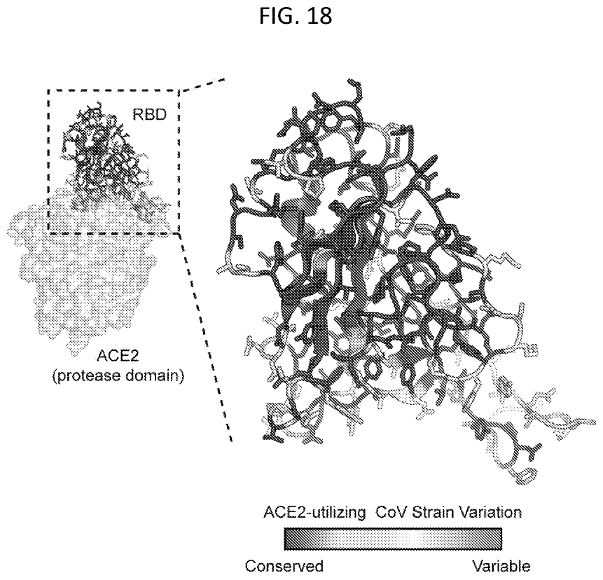

The affinities of the decoy receptor sACE2₂.v2.4 were determined for purified RBDs from the S proteins of five coronaviruses from Rhinolophus bat species (isolates LYRa 11, Rs4231, Rs7327, Rs4084 and RsSHC014) and two human coronaviruses, SARS-CoV-1 and SARS-CoV-2. These viruses fall within a common clade of betacoronaviruses that use ACE2 as an entry receptor (Letko et al., Nat Microbiol. 11, 1860, 2020). They share close sequence identity within the RBD core while variation is highest within the functional ACE2 binding site (FIGS. 18 and 19), possibly due to a co-evolutionary ‘arms race’ with polymorphic ACE2 sequences in ecologically diverse bat species (Frank et al., bioRxiv. 5, 562, 2020). Affinity was measured by biolayer interferometry (BLI), with sACE2₂ (a.a. S19-G732) fused at the C-terminus with the Fc moiety of human IgG1 immobilized to the sensor surface and monomeric 8his-tagged RBD used as the soluble analyte. This arrangement excludes avidity effects, which otherwise cause artificially tight (picomolar) apparent affinities whenever dimeric sACE2₂ in solution is bound to immobilized RBD decorating an interaction surface. Wild type sACE2₂ bound all the RBDs with affinities ranging from 16 nM for SARS-CoV-2 to 91 nM for LYRa 11, with median affinity 60 nM (Table 6). The measured affinities for the RBDs of SARS-CoV-1 and SARS-CoV-2 are comparable to published data (Wrapp et al., Science, eabb2507, 2020; Chan et al., Science. 4, eabcO870, 2020; Shang et al., Nature. 382, 1199, 2020; Kirchdoerfer et al., Sci. Rep. 8, 15701, 2018; Li et al., EMBO J 24, 1634-1643, 2005). Engineered sACE2₂.v2.4 displayed large increases in affinity for all the RBDs, with K_{DS} ranging from 0.4 nM for SARS-CoV-2 to 3.5 nM for isolate Rs4231, with median affinity less than 2 nM (Table 6). The approximate 35-fold affinity increase of the engineered decoy applies universally to coronaviruses in the test panel and the molecular basis for affinity enhancement must therefore be grounded in common attributes of RBD/ACE2 recognition.

TABLE 6
BLI kinetics for immobilized sACE2₂-IgG1 binding to coronavirus RBDs

| | Wild type sACE2₂-IgG1 ^{b} | | | | sACE2₂.v2.4-IgG1 | | | |
|---|---|---|---|---|---|---|---|---|
| | kₒₙ | k_{off} | K_{D} | | kₒₙ | k_{off} | K_{D} | |
| CoV strain^{a} | (M⁻¹ s⁻¹) | (s⁻¹) | (nM) | χ^{2 c} | (M⁻¹ s⁻¹) | (s⁻¹) | (nM) | χ² |
| LYRa11 | 8.7 × 10⁵ | 7.9 × 10⁻² | 91 | 0.12 | 1.4 × 10⁶ | 2.5 × 10⁻³ | 1.8 | 0.10 |
| Rs7327 | 6.4 × 10⁵ | 4.0 × 10⁻² | 63 | 0.25 | 9.8 × 10⁵ | 1.8 × 10⁻³ | 1.9 | 0.11 |
| Rs4231 | 3.2 × 10⁵ | 2.2 × 10⁻² | 69 | 0.04 | 4.5 × 10⁵ | 1.6 × 10⁻³ | 3.5 | 0.10 |
| Rs4084 | 2.9 × 10⁵ | 2.5 × 10⁻² | 85 | 0.24 | 4.8 × 10⁵ | 1.5 × 10⁻³ | 3.1 | 0.10 |
| RsSHC014 | 8.8 × 10⁵ | 2.6 × 10⁻² | 29 | 0.20 | 1.6 × 10⁶ | 2.0 × 10⁻³ | 1.3 | 0.29 |
| SARS-1 | 6.6 × 10³ | 1.2 × 10⁻⁴ | 58 | 0.03 | 3.0 × 10³ | 5.6 × 10⁻⁶ | 2.1 | 0.03 |
| SARS-2 | 1.4 × 10⁶ | 8.1 × 10⁻³ | 16 | 0.25 | 6.6 × 10⁵ | 2.8 × 10⁻⁴ | 0.4 | 0.09 |
| SARS-2 | 2.0 × 10⁶ | 9.0 × 10⁻² | 46 | 0.67 | 4.3 × 10⁶ | 4.0 × 10⁻³ | 0.9 | 0.71 |
| (Y449K) | | | | | | | | |
| SARS-2 | 2.4 × 10⁶ | 5.4 × 10⁻³ | 2.3 | 0.43 | 3.3 × 10⁶ | 2.8 × 10⁻⁴ | 0.1 | 0.23 |
| (N501W) | | | | | | | | |
| SARS-2 | 2.2 × 10⁶ | 1.8 × 10⁻³ | 0.8 | 0.15 | 3.6 × 10⁵ | 1.1 × 10⁻⁴ | <0.1 | 0.24 |
| (N501Y) | | | | | | | | |

^{a} Purified RBDs at 5 to 7 concentrations were used as the soluble analytes.
^{b} IgG1-Fc fused sACE2₂ was immobilized to anti-human IgG Fc capture biosensors.
^{c} χ² values represent the goodness of curve fitting. Acceptable values were considered less than 3.

### A Deep Mutational Scan of the RBD in the Context of Full-Length S Reveals Residues in the ACE2 Binding Site are Mutationally Tolerant

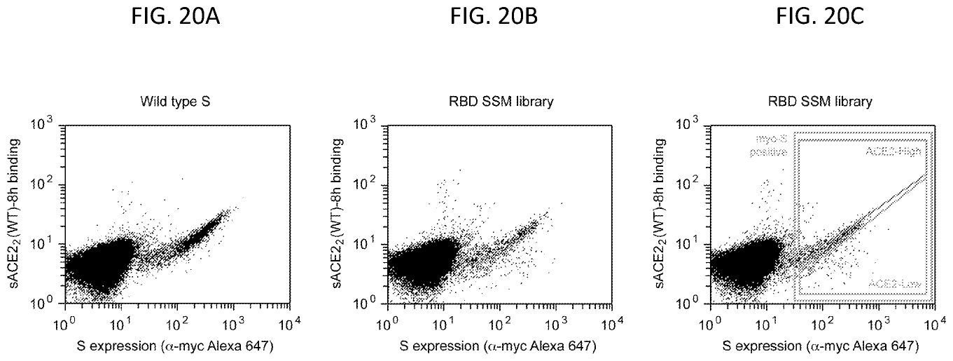

To explore potential sequence diversity in S of SARS-CoV-2 that may act as a ‘reservoir’ for drug resistance, the mutational tolerance of the RBD was evaluated by deep mutagenesis (Fowler and Fields, Nat. Methods. 11, 801-807, 2014). Saturation mutagenesis was focused to the RBD (a.a. C336-L517) of full-length S tagged at the extracellular N-terminus with a c-myc epitope for detection of surface expression. The spike library, encompassing 3,640 single amino acid substitutions, was transfected in human Expi293F cells under conditions where cells typically acquire no more than a single sequence variant (Heredia et al., J. Immunol. 200, ji 1800343-3839, 2018; Park et al., J Biol Chem. 294, 4759-4774, 2019). The culture was incubated with wild type, 8his-tagged, dimeric sACE2₂ at a sub-saturating concentration (2.5 nM). Bound sACE2₂-8h and surface-expressed S were stained with fluorescent antibodies for flow cytometry analysis (FIG. 20A). Compared to cells expressing wild type S, the library was poorly expressed, indicating many mutations are deleterious for folding and expression. A cell population was clearly discernable expressing S variants that bind ACE2 with decreased affinity (FIG. 20B). After gating for c-myc-positive cells expressing S, cells with high and low levels of bound sACE2₂ were collected by fluorescence-activated cell sorting (FACS), called the ACE2-High and ACE2-Low populations, respectively (FIG. 20C). Both the expression and sACE2₂ binding signals decreased over minutes to hours during sorting, possibly due to shedding of the S1 subunit. Cells were therefore collected and pooled from three separate FACS experiments for a combined 8 hours sort time.

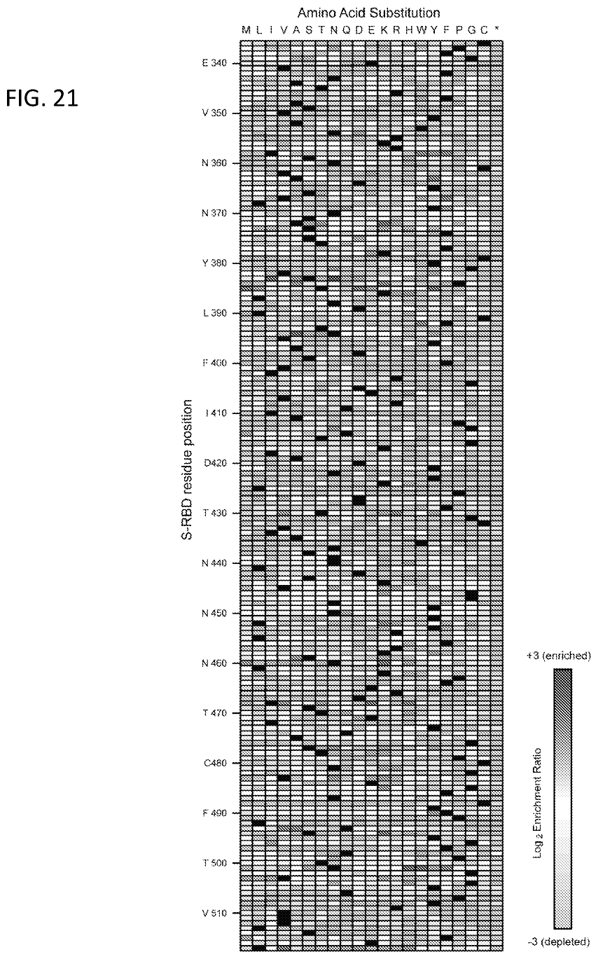

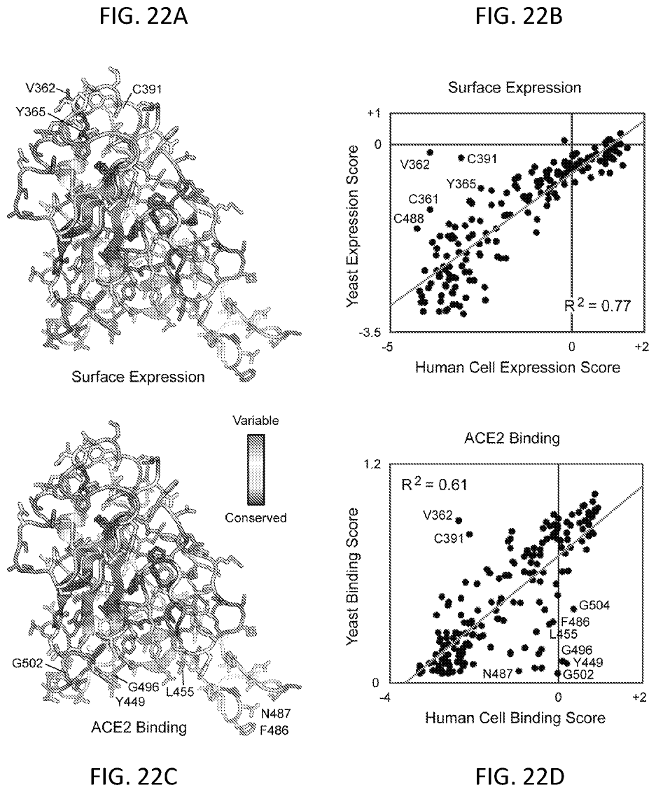

Transcripts in the sorted cells were Illumina sequenced and compared to the naive plasmid library to determine an enrichment ratio for each amino acid substitution (Fowler et al., Bioinformatics. 27, 3430-3431, 2011). Mutations in S that express and bind ACE2 tightly are selectively enriched in the ACE2-High sort (FIG. 21); mutations that express but have reduced ACE2 binding are selectively enriched in the ACE2-Low sort; and mutations that are poorly expressed are depleted from both sorted populations. Positional conservation scores were calculated by averaging the log 2 enrichment ratios for each of the possible amino acids at a residue position. By adding conservation scores for both the ACE2-High and ACE2-Low sorts a score was derived for surface expression, which shows that the hydrophobic RBD core is tightly conserved for folding and trafficking of the viral spike (FIG. 22A). By comparison, residues on the exposed RBD surface are mutationally permissive for S surface expression. This matches the mutational tolerance of proteins generally.

For tight ACE2 binding (e.g., S variants in the ACE2-High population), conservation increases for RBD residues at the ACE2 interface, yet mutational tolerance remains high (FIG. 22C). The sequence diversity observed among natural betacoronaviruses, which display high diversity at the ACE2 binding site, is therefore replicated in the deep mutational scan, which predicts the SARS-CoV-2 spike tolerates substantial genetic diversity at the receptor-binding site for function. From this accessible sequence diversity SARS-CoV-2 might feasibly mutate to acquire resistance to monoclonal antibodies or engineered decoy receptors targeting the ACE2-binding site.

### Comparison to a Deep Mutational Scan of the Isolated RBD by Yeast Surface Display

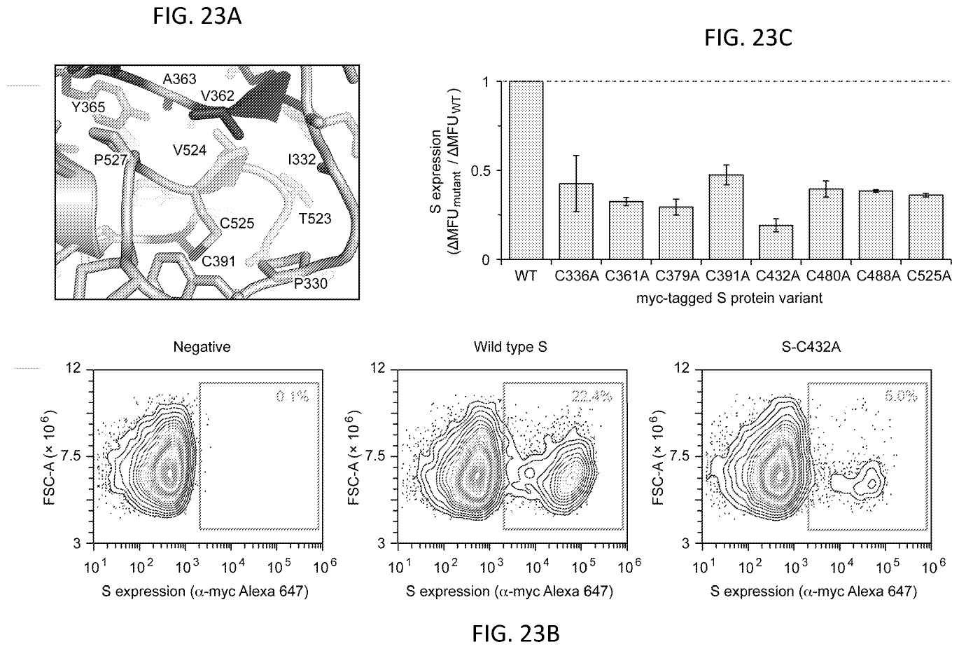

Two deep mutational scans have been reported for the isolated RBD displayed on the surface of yeast (Starr et al., bioRxiv, 2020.06.17.157982, 2020; Linsky et al., bioRxiv, 2020.08.03.231340, 2020). The data described herein, from a selection of full-length S expressed in human cells, is compared to the publicly accessible Starr et al. data set. Important residues within the RBD for surface expression of full-length spike in human cells are closely correlated with data from yeast surface display of the isolated RBD (FIG. 22B), with the exception of a notable region. The surface of the RBD opposing the ACE2-binding site (e.g., V362, Y365 and C391) is free to mutate for yeast surface display, but its sequence is constrained in the present experiments; this region of the RBD is buried by connecting structural elements to the global fold of an S subunit in the closed-down conformation (this is the dominant conformation for S subunits and is inaccessible to receptor binding) (Walls et al., Cell, 2020), doi:10.1016/j.cell.2020.02.058; Wrapp et al., Science, eabb2507, 2020; Cai et al., Science. 369, 1586, 2020; Yao et al., Cell 183(3), 730-738.e13, 2020). Targeted mutagenesis was used to individually test alanine substitutions for all the cysteines in the RBD (FIG. 23). All cysteine-to-alanine mutations severely diminished S surface expression in Expi293F cells, including C391 Å and C525 Å on the RBD ‘backside’ that were neutral in the yeast display scan. These differences demonstrate that there are tighter sequence constraints on the RBD in the context of a full spike expressed at a human cell membrane, yet overall the two data sets closely agree.

For binding to dimeric sACE2₂, interface residues were more tightly conserved in the Starr et al. data set (FIG. 22D), possibly a consequence of three differences between the deep mutagenesis experiments. First, the selections for ACE2 binding of S variants at the plasma membrane appears to primarily reflect mutational effects on surface expression, which is almost certainly more stringent in human cells. Yeast permit many poorly folded proteins to leak to the cell surface (Rocklin et al., Science. 357, 168-175, 2017). Second, the yeast selections were conducted at multiple sACE2 concentrations from which apparent K_{D} changes were computed; the Starr et al. data in this regard is very comprehensive. Due to the long sort times required for the human cell libraries where only a small fraction of cells express spike, sorting was performed at a single sACE2₂ concentration that cannot accurately capture a range of different binding affinities quantitatively. Third, dimeric sACE2₂ may geometrically complement trimeric S densely packed on a human cell membrane, such that avidity masks the effects of affinity-reducing mutations. Nonetheless, there is overall agreement that ACE2 binding often persists following mutations to the RBD surface, and these data simply suggest mutational tolerance may be even greater than that already observed by Starr et al.

A Screen for S Variants that Preferentially Bind Wild Type ACE2 Over the Engineered Decoy

Having shown that the ACE2-binding site of SARS-CoV-2 protein S tolerates many mutations, it was investigated whether mutations might therefore be found that confer resistance to the engineered decoy sACE2₂.v2.4. Resistance mutations are anticipated to lose affinity for sACE2₂.v2.4 while maintaining binding to the wild type receptor, and are most likely to reside in the RBD where physical contacts are made. Similar reasoning formed the foundation of a deep mutagenesis-based selection of the isolated RBD by yeast surface display to find escape mutations to monoclonal antibodies, and the results were predictive of escape mutations in pseudovirus growth selections (Greaney et al., bioRxiv, 2020.09.10.292078, 2020).

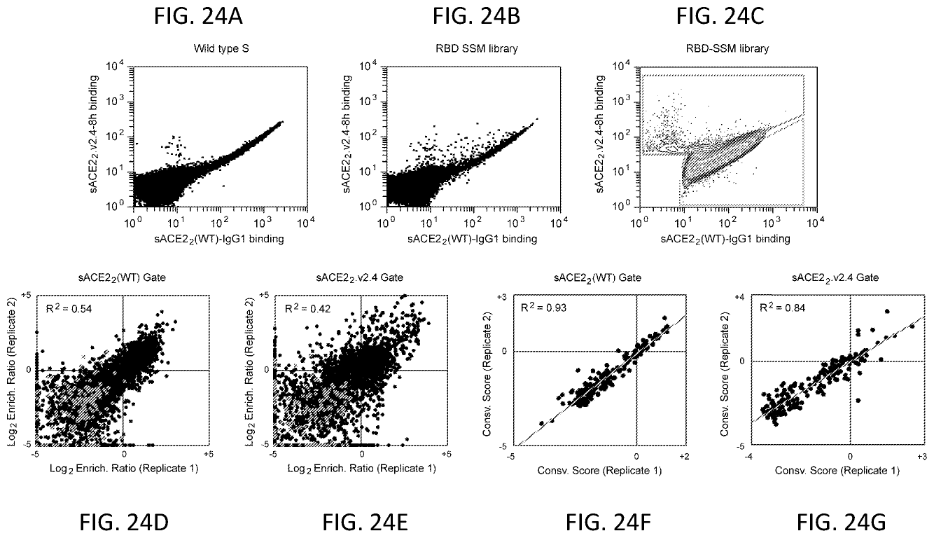

To address whether escape mutations from the engineered decoy might be found in the RBD, the S protein library was repurposed for a specificity selection. Cells expressing the library, encoding all possible substitutions in the RBD, were co-incubated with wild type sACE2₂ fused to the Fc region of IgG1 and 8his-tagged sACE2₂.v2.4 at concentrations where both proteins bind competitively (Chan et al., Science. 4, eabc0870, 2020). It was immediately apparent from flow cytometry of the Expi293F culture expressing the S library that there were cells expressing S variants shifted towards preferential binding to sACE2₂.v2.4, but no significant population with preferential binding to the wild type receptor (FIGS. 24A-24B). Cells expressing S variants that might preferentially bind sACE2₂(WT)-IgG1 or sACE2₂.v2.4 were gated and collected by FACS (FIG. 24C), followed by deep sequencing of S transcripts to determine enrichment ratios. There was close agreement between two independent replicate experiments (FIGS. 24D-24G). Most RBD mutations were depleted following sorting, consistent with deleterious effects on S folding and expression.

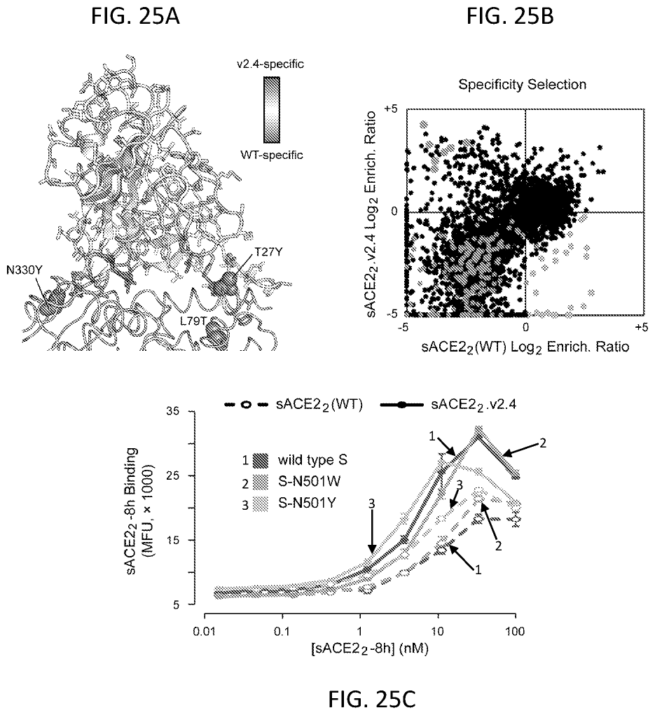

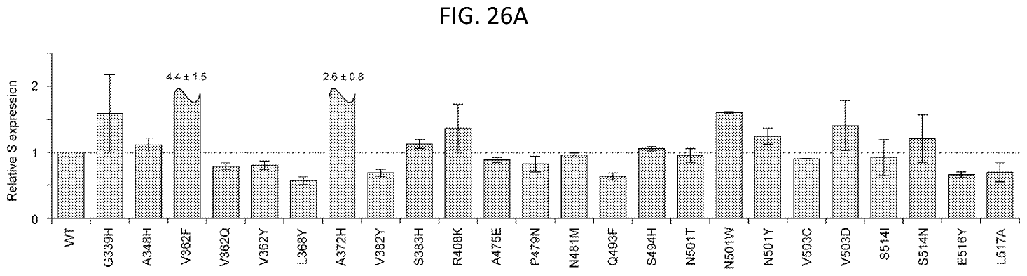

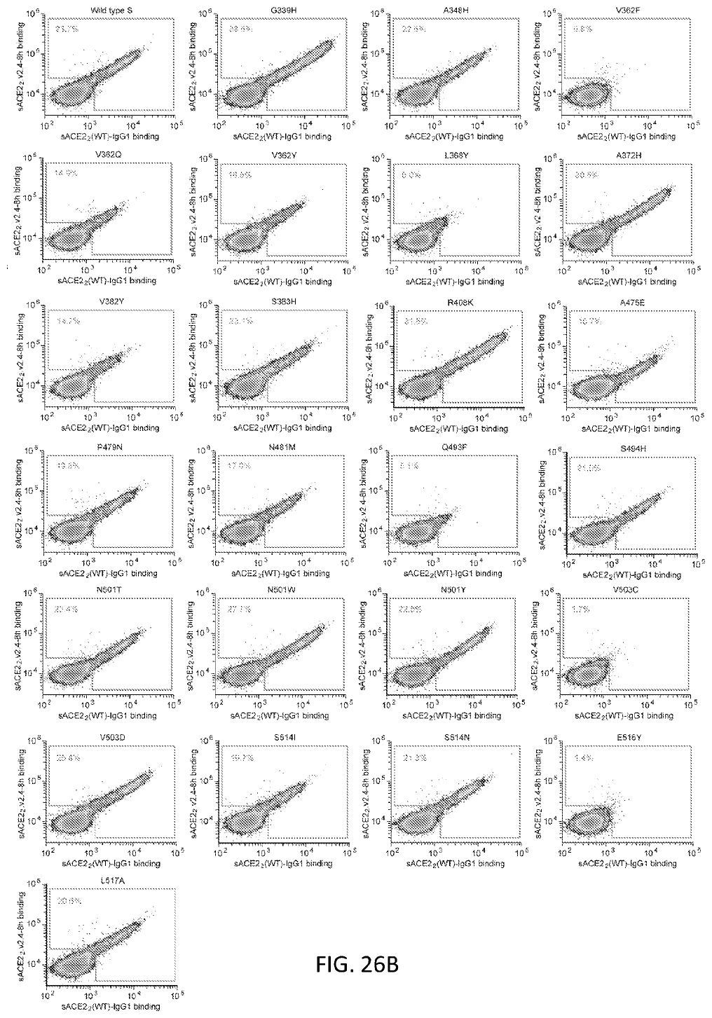

Soluble ACE2₂.v2.4 has three mutations from wild type ACE2: T27Y buried within the RBD interface, and L79T and N330Y at the interface periphery (FIG. 25A). A substantial number of mutations in the RBD of S were selectively enriched for preferential binding to sACE2₂.v2.4 (FIG. 25B, upper-left quadrant). While sACE2₂.v2.4-specificity mutations could be found immediately adjacent to the sites of engineered mutations in ACE2 (in particular mutations to S-F486 adjacent to ACE2-L79 and S-T500 adjacent to ACE2-N330), major hot spots for sACE2₂.v2.4-specificity mutations also mapped to RBD loop 498-506 contacting the region where the ACE2-α1 helix packs against a β-hairpin motif (FIG. 25A). By comparison, there were no hot spots in the RBD for sACE2₂(WT)-specificity mutations. Indeed, only a small number of mutations were selectively enriched for preferential binding to wild type receptor (FIG. 25B), and the abundance of these putative wild type-specific mutations barely rose above the expected level of noise in the deep mutagenesis data. In this competition assay, S binding to wild type sACE2₂ is therefore more sensitive to RBD mutations than is S binding to engineered sACE2₂.v2.4.

To determine whether the potential wild type ACE2-specific mutations found by deep mutagenesis are real as opposed to false predictions due to data noise, 24 mutants of S selectively enriched in the wild type-specific gate by targeted mutagenesis were tested (blue data points in FIG. 25B). Only minor shifts towards binding wild type sACE2₂ were observed (FIG. 26). Two S mutants were investigated further in sACE2₂ titration experiments, N501W and N501Y, which both retained high receptor binding and displayed small shifts towards wild type sACE2₂ in the competition experiment. N501 of S is located in the 498-506 loop and its substitution to large aromatic side chains might alter the loop conformation to cause steric strain with nearby ACE2 mutation N330Y in sACE2₂.v2.4. After titrating the concentrations of 8his-tagged sACE2₂(WT) and sACE2₂.v2.4 and measuring bound protein to S-expressing cells by flow cytometry, it was found S-N501W and S-N501Y do show enhanced specificity for wild type sACE2₂, but the effect is small and sACE2₂.v2.4 remains the stronger binder (FIG. 25C); these mutations therefore will not confer resistance in the virus to the engineered decoy.

Dimeric sACE2₂ binds avidly to S protein on a membrane surface; avid interactions are also observed between sACE2₂ and spikes on authentic SARS-CoV-2 in infection assays (Chan et al., Science. 4, eabc0870, 2020). BLI kinetics measurements, in which immobilized sACE2₂-IgG1 interacts with monomeric RBD, were used to determine how the observed changes in avid sACE2₂ binding to S-expressing cells translate to changes in monovalent affinity. Both N501W and N501Y mutants of SARS-CoV-2 RBD displayed increased affinity for wild type ACE2 and engineered ACE2.v2.4, with larger affinity gains in favor of the wild type receptor (Table 6). This aligns with the flow cytometry data indicating a small shift in specificity towards wild type ACE2, but not enough to escape the engineered decoy. By comparison, multiple independent escape mutations are readily found in S of SARS-CoV-2 that diminish the efficacy of monoclonal antibodies by many orders of magnitude (Baum et al., Science, eabd0831, 2020; Greaney et al., bioRxiv, 2020.09.10.292078, 2020).

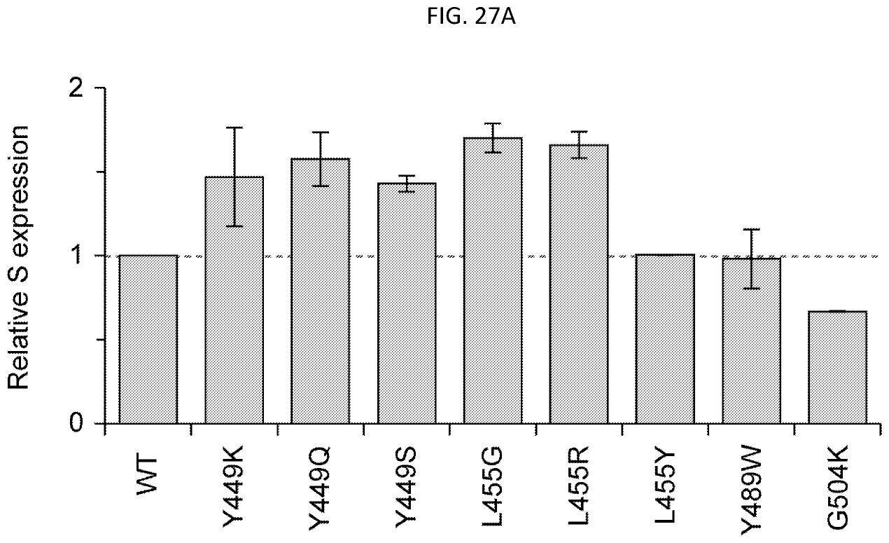

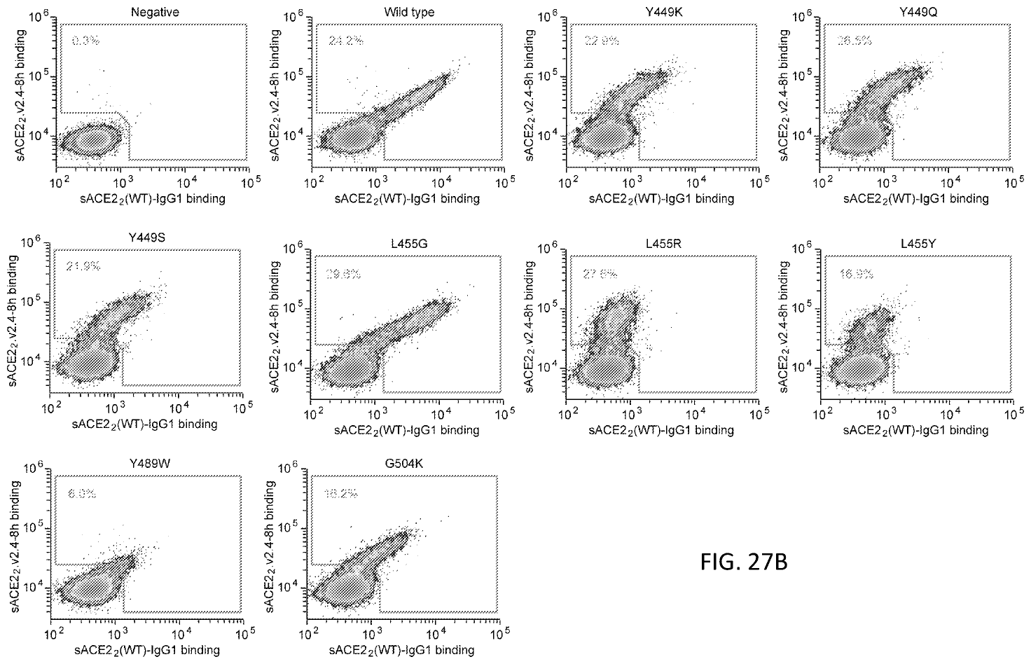

Finally, 8 representative mutations to S predicted from the deep mutational scan to increase specificity towards sACE2₂.v2.4 (FIG. 25B) were cloned and 7 were found to have large shifts towards preferential sACE2₂.v2.4 binding in the competition assay (FIG. 27). These S mutations were Y449K/Q/S, L455G/R/Y and G504K. The basis for why the mutations increase specificity towards engineered sACE2₂.v2.4 is ambiguous, since RBD residues Y449, L455, and G504 are not in direct contact with engineered sites of the receptor. BLI kinetics between immobilized sACE2₂-IgG1 and monomeric RBD as the analyte showed reduced affinity of a representative mutant, RBD-Y449K, to both wild type and engineered sACE2₂ (Table 6). However, affinity changes in the picomolar range for sACE2₂.v2.4 are hidden during avid binding to full-length S-Y449K at the cell surface, whereas avid binding of wild type sACE2₂ to S-Y449K (with affinity measured by BLI in the moderate nanomolar range) is substantially reduced. This finding might explain why the competition selection found many mutations that shift specificity towards engineered sACE2₂.v2.4, as mutations causing small decreases in affinity may have larger effects on avid binding of the weaker-bound wild type receptor.

Overall, validation by targeted mutagenesis confirms that the selection can successfully find mutations in S with altered specificity. The inability to find mutations in the RBD that impart high specificity for the wild type receptor means such mutations are rare or may not even exist, at least within the receptor-binding domain where direct physical contacts with receptors occur. Mutations elsewhere having long-range conformational effects cannot be excluded. Engineered, soluble decoy receptors therefore live up to their promise as broad therapeutic candidates against which a virus cannot easily escape.

### DISCUSSION

The allure of soluble decoy receptors is that the virus cannot easily mutate to escape neutralization. Mutations that reduce affinity of the soluble decoy will likely also decrease affinity for the wild type receptor on host cells, thereby coming at the cost of diminished infectivity and virulence. However, this hypothesis has not been rigorously tested, and since engineered decoy receptors differ from their wild type counterparts, even if by just a small number of mutations, it is possible a virus may evolve to discriminate between the two. Here, it is demonstrated that an engineered decoy receptor for SARS-CoV-2 broadly binds with low nanomolar K_{D} the spikes of SARS-associated betacoronaviruses that use ACE2 for entry, despite high sequence diversity within the ACE2-binding site. Mutations in S that confer high specificity for wild type ACE2 were not found in a comprehensive screen of all substitutions within the RBD. The engineered decoy receptor is therefore broad against zoonotic ACE2-utilizing coronaviruses that may spill over from animal reservoirs in the future and against variants of SARS-CoV-2 that may arise as the current COVID-19 pandemic rages on. It is unlikely that decoy receptors will need to be combined in cocktail formulations, as is required for monoclonal antibodies or designed miniprotein binders to prevent the rapid emergence of resistance (Baum et al., Science, eabd0831, 2020; Cao et al., Science, eabd9909, 2020).

Soluble decoy receptors have proven effective in the clinic, especially for modulating immune responses. Etanercept (trade name Enbrel®; soluble TNF receptor), aflibercept (Eylea®; a soluble chimera of VEGF receptors 1 and 2) and abatacept (Orencia®; soluble CTLA-4) are just three examples of soluble receptors that have profoundly impacted the treatment of human disease (Usmani et al., PLoS ONE. 12, e0181748, 2017), yet no soluble receptors for a viral pathogen are approved drugs. There are two main reasons for this. First, the affinity of entry receptors for viral glycoproteins is often moderate to low, which reduces neutralization potency compared to affinity-matured monoclonal antibodies. For SARS-CoV-2, this problem has been solved by engineering ACE2 to have picomolar affinity for viral S (Chan et al., Science. 4, eabcO870, 2020; Glasgow et al., bioRxiv, 2020.07.31.231746, 2020; Higuchi et al., bioRxiv, 2020.09.16.299891, 2020). Second, virus entry receptors have endogenous functions for normal physiology and their soluble counterparts may impact this normal physiology to exert unacceptable toxicity. For example, the entry receptor for human cytomegalovirus is a growth factor receptor, and growth factor interactions had to be knocked out to make a virus-specific decoy suitable for in vivo administration (Park et al., PLoS Pathog. 16, e1008647, 2020). However, ACE2 in this regard is different and its endogenous activity—the catalytic conversion of vasoconstrictive and inflammatory peptides of the renin-angiotensin and kinin systems—may be of direct benefit for addressing COVID-19 symptoms. During infection, ACE2 activity is downregulated and the renin-angiotensin system becomes imbalanced, possibly driving aspects of acute-respiratory distress syndrome (ARDS) that cause patients to require mechanical ventilation (Imai et al., Nature. 436, 112-116, 2005; Treml et al., Crit. Care Med. 38, 596-601, 2010; Verdecchia et al., Eur J Intern Med. 76, 14-20, 2020). Administration of recombinant, soluble ACE2 rescues lost biochemical activity, with potential protective properties for the pulmonary and cardiovascular systems that include decreased lung elastance, increased blood oxygenation, reduced hypertension and diminished fluid accumulation in the lungs due to proteolytic conversion of angiotensin and bradykinin peptides (Imai et al., Nature. 436, 112-116, 2005; Treml et al., Crit. Care Med. 38, 596-601, 2010; Wang et al., Pulm Pharmacol Ther. 58, 101833, 2019; Chung et al., EBioMedicine. 58, 102907-102907, 2020; Johnson et al., PLoS ONE. 6, e20828, 2011; Liu et al., Kidney Int. 94, 114-125, 2018; Garvin et al., Elife. 9, e59177, 2020). Soluble, wild type ACE2₂ has been developed as a drug for ARDS with an acceptable safety profile in humans (Haschke et al., Clin Pharmacokinet. 52, 783-792, 2013; Khan et al., Crit Care. 21, 234, 2017) and is currently under evaluation in a clinical trial by Apeiron. Engineered, high affinity sACE2₂ decoys, most likely as fusions with immunoglobulin Fc for increased serum stability (Lei et al., Nat Commun. 11, 2070, 2020; Liu et al., Kidney Int. 94, 114-125, 2018; Iwanaga et al., bioRxiv, 2020.06.15.152157, 2020), represent next generation therapeutics with dual mechanisms of action: (i) potent virus neutralization due to high affinity blockade of the viral spike and (ii) proteolytic turnover of peptide hormones for direct relief of COVID-19 symptoms.

### Example 5

This example evaluates pharmacokinetics (PK) of sACE2.v2.4 in mice. The results demonstrate that serum half-life of sACE2.v2.4 following IV administration is increased by fusion to the Fc moiety of human IgG1. The fusion protein is proteolysed to produce long-lived IgG1 fragments that persist beyond 7 days, whereas the ACE2 moiety rapidly disappears within hours. By delivering sACE2₂.v2.4-IgG1 directly to the lungs via intratracheal (IT) administration or nebulization, the protein remains at high levels in lung tissue for at least 4 hours with minimal proteolytic degradation. These results demonstrate that direct lung delivery of high affinity sACE2 derivatives is a viable alternative to IV infusion, and offers possible benefits for out-patient clinical care.

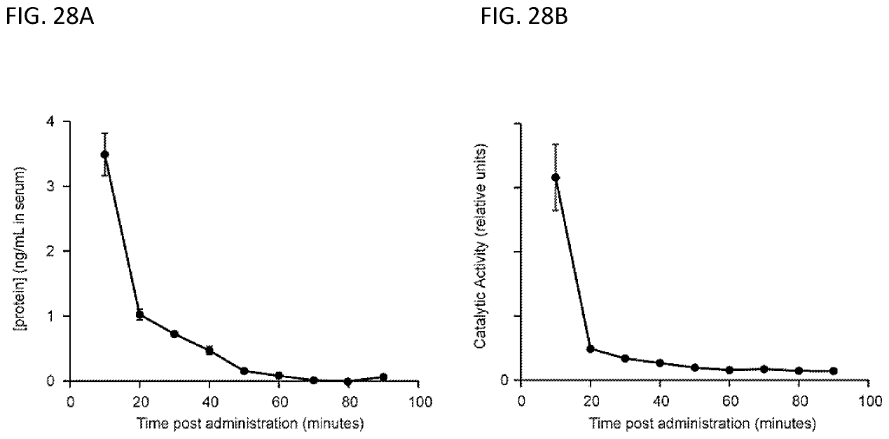

When wild type human sACE2₂ is administered intraperitoneally in mice, it has a serum half-life of 8.5 hours (Wysocki et al., Hypertension 55, 90-98, 2010), but this is influenced by resorption kinetics into the blood, which is typically delayed by hours for macromolecules (Shoyaib et al., Pharmaceut Res. 37, 12, 2020). When sACE2₂.v2.4 (0.5 mg/kg) without a fusion partner was injected into the tail veins of male and female mice, the protein was rapidly cleared with a serum half-life estimated to be under 10 minutes, measured by ACE2 ELISA (FIG. 28A) and ACE2 catalytic activity in serum (FIG. 28B). This is much shorter than the serum half-life of wild type sACE2₂ in humans (2 to 3 hours) (Haschke et al., Clin Pharmacokinet 52, 783-792, 2013). No toxicity was observed when sACE2₂.v2.4 was IV administered twice daily at 0.5 mg/kg for five days (days 0, 1, 2, 3, and 4). Mice were euthanized on day 7 and blood chemistry, hematology, and tissue pathology showed no differences with mock treated mice.

To increase serum half-life, a fusion of sACE2₂ to IgG1 Fc was tested. While other groups have explored fusions of sACE2₂ to IgG1 mutants (Iwanaga et al., Biorxiv, in press, doi:10.1101/2020.06.15.152157) or IgG4 Fc (Svilenov et al., Biorxiv, in press, doi:10.1101/2020.12.06.413443) to dampen interactions with pro-inflammatory FcγRs, this study used unmodified IgG1 (isoallotype nGlml) to recruit effector functions that have been shown in anti-SARS-CoV-2 mAbs to be necessary for optimum protection (Schafer et al., J ExpMed. 218 (2020), doi:10.1084/jem.20201993). Published PK data on IgG1 fusions of sACE2₂ are mixed. While there is unambiguous evidence that a murine sACE2₂-IgG1 fusion persists for days in mice (Liu et al., Kidney Int. 94, 114-125, 2018), results on human sACE2₂-IgG1 fusions are contradictory. Two reports using an ELISA to detect the human IgG1 moiety indicated sACE2₂-IgG1 has a serum half-life of days (Iwanaga et al., Biorxiv, in press, doi:10.1101/2020.06.15.152157; Lei et al., Nat Commun. 11, 2070, 2020), but another study using an ELISA to detect the ACE2 moiety reported rapid clearance within hours (Higuchi et al., Biorxiv, in press, doi:10.1101/2020.09.16.299891). Only one published report detected both parts of the fusion protein, using an anti-ACE2 capture antibody with an anti-IgG1 detection antibody to measure a long serum half-life of the human fusion protein in mice (Liu et al., Int J Biol Macromol. 165, 1626-1633, 2020). The reasons for the discrepancies are unclear, but possibly indicate cleavage of the fusion protein to produce fragments of differing serum stability.

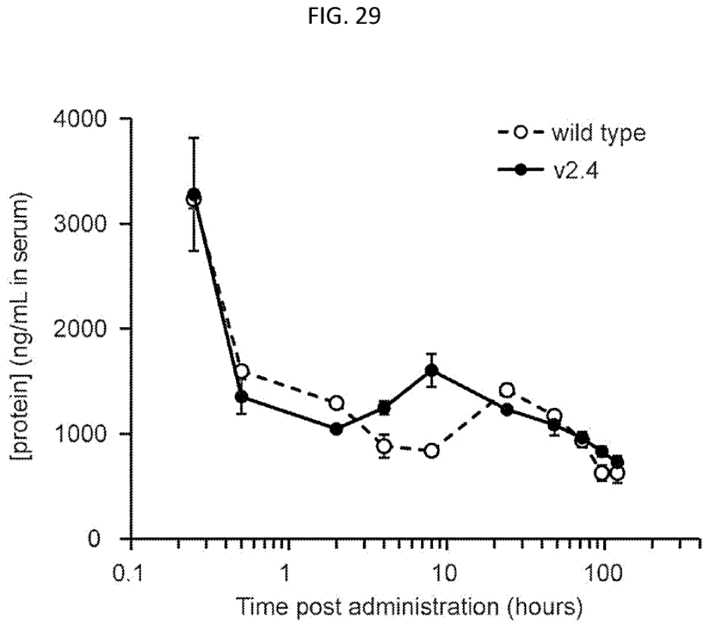

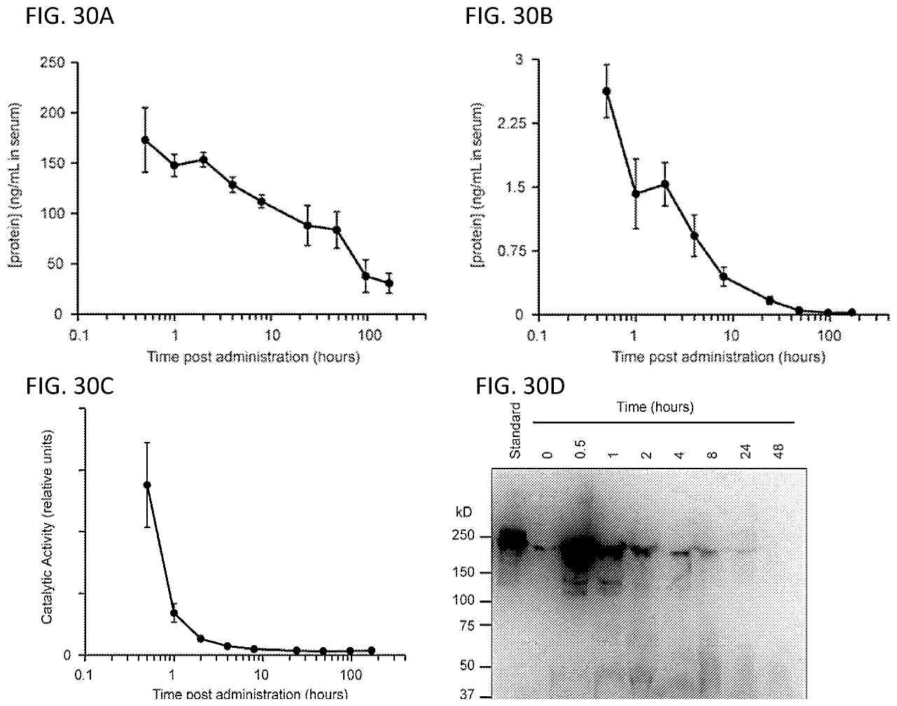

Using an ELISA for human IgG1, both wild type sACE2₂-IgG1 and sACE2₂.v2.4-IgG1 (SEQ ID NO: 11) showed equivalent serum PK after IV administration (2.0 mg/kg) in male mice, with protein persisting for over 7 days (FIG. 29). It was therefore concluded that the three mutations in the high affinity sACE2₂.v2.4 variant (T27Y, L79T, and N330Y) did not substantially change PK, consistent with a previous study of another modified sACE2 derivative (Higuchi et al., Biorxiv, in press, doi:10.1101/2020.09.16.299891). Serum components could not be further characterized due to insufficient material, consequently another PK study was conducted in both male and female mice to more thoroughly track how sACE2₂.v2.4-IgG1 changes in the serum with time. Again, human IgG1 protein persisted for days in the serum (FIG. 30A), yet the ACE2 moiety was rapidly cleared within 24 hours based on an ACE2 ELISA (FIG. 30B). Measurement of ACE2 catalytic activity revealed even faster decay (FIG. 30C). Immunoblot for human IgG1 confirmed that the fusion protein was being proteolyzed in mouse blood to liberate long-lived IgG1 fragments (FIG. 30D). Overall, fusion of sACE2₂.v2.4 to IgG1 Fc provided only a modest increase in serum stability. No toxicity was observed in mice IV administered sACE2₂.v2.4-IgG1 (2.0 mg/kg), followed up 7 days later with blood chemistry, hematology, and tissue pathology analysis.

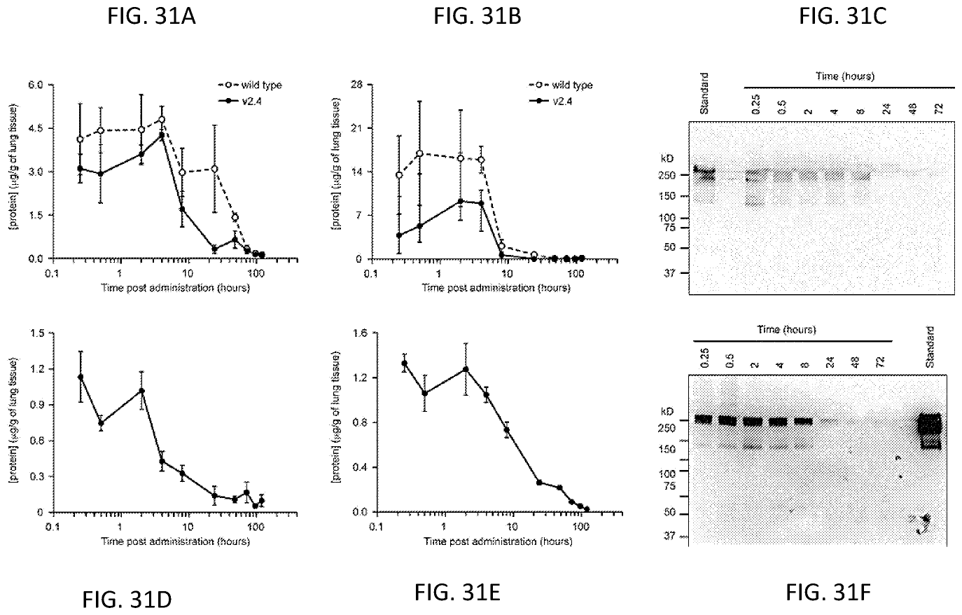

To improve upon serum PK observed following IV administration, a study was performed to deliver the protein directly to the respiratory tract, which is the primary site of SARS-CoV-2 infection. Following IT delivery (1.0 mg/kg), sACE2₂.v2.4-IgG1 was found to persist at high levels in the lungs for at least 4 hours by ACE2 ELISA, human IgG1 ELISA, and anti-human IgG1 immunoblot (FIGS. 31A-31C). Levels of sACE2₂.v2.4-IgG1 absorbed into the blood were too low for detection. As observed for IV administration, wild type sACE2₂-IgG1 and sACE2₂.v2.4-IgG1 had equivalent PK in the lungs (within experimental error) following IT delivery. Administration of sACE2₂.v2.4-IgG1 by inhalation was further investigated. In this study, the protein was nebulized for 30 minutes into a chamber holding the mice. While doses in the nebulizer-holding chamber were below that achieved through IT administration, it was nonetheless observed that sACE2₂.v2.4-IgG1 remained high and relatively constant for 4 hours, as measured by ACE2 ELISA, human IgG1 ELISA, and immunoblot (FIGS. 31D-31F). Direct delivery to the respiratory tract achieved high levels of protein in the lung tissue with minimal degradation for over 4 hours. The different PK profiles based on route of administration (e.g., protein delivered directly to the lungs persists for hours but does not reach detectable levels in plasma, whereas IV delivered protein achieves high but short lived plasma concentrations) offer clinical opportunities to treat patients in different ways, possibly based on how advanced disease is or whether infection is systemic, or to treat patients using both IV and either IT or inhalation routes of administration.

### Example 6

This example describes experiments performed using SARS-CoV-2 pseudovirus to evaluate whether modified ACE2 polypeptides are capable of blocking virus entry into cells.

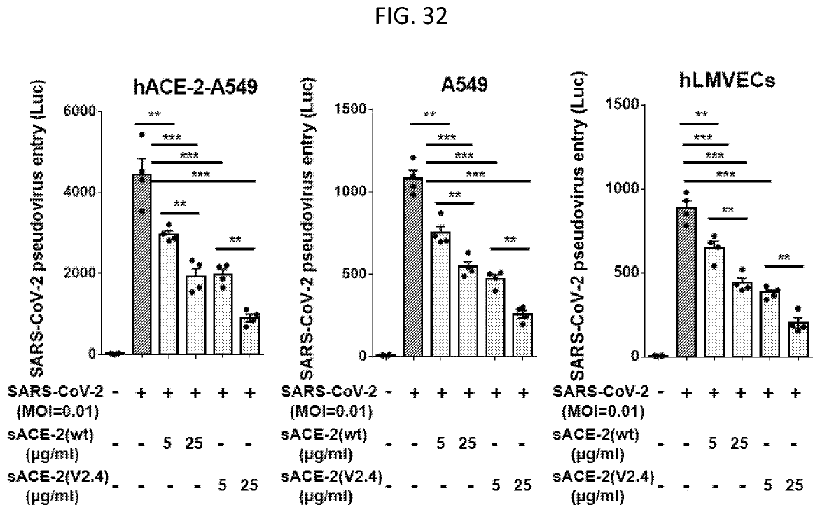

Human A549 lung epithelial cells over-expressing the ACE2 receptor, human A549 lung epithelial cells, and human lung endothelial cells were incubated with a VSV-SARS-CoV-2-luciferase-pseudotype virus and the wild-type sACE2₂-IgG1 or the engineered sACE2₂.v2.4-IgG1 peptides at concentrations of 0, 5 or 25 μg/ml. Each experiment contained a no virus control; all other samples contained the virus at an MOI of 0.01. Cells were harvested and the extent of viral entry was quantified based on expression of the luciferase reporter (FIG. 32). Engineered sACE2₂.v.2.4-IgG1 had superior protection against entry of the SARS-CoV-2 pseudovirus into human lung epithelial cells and human endothelial cells.

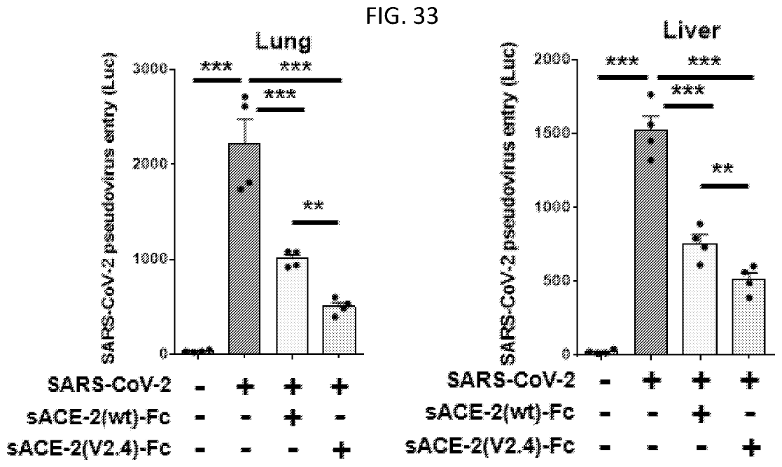

In a second study, K18-hACE2 transgenic mice, which express the human ACE2 receptor in epithelial cells, were injected intravenously with either wild-type sACE2₂-IgG1 or sACE2₂.v2.4-IgG1 and intraperitoneally with the VSV-SARS-CoV-2-luciferase-pseudotype virus. The lung and the liver were harvested at 24 hours and the extent of viral entry was quantified by luciferase activity (FIG. 33). Engineered sACE2₂.v.2.4-IgG1 achieved superior protection against SARS-CoV-2 pseudotype virus entry into the lung and liver in human ACE2-expressing mice.

Taken together, these results demonstrate that the v2.4 derivative of soluble ACE2 more effectively blocks SARS-CoV-2 pseudovirus entry into cells expressing human ACE2, both in tissue culture and in an animal model.

### Example 7

This example describes a study to investigate whether sACE2₂.v2.4-IgG1 exhibits protective and/or therapeutic benefits against SARS-CoV-2-induced lung vascular leakage in a mouse model of COVID-19. While particular methods are provided, one of skill in the art will recognize that methods that deviate from these specific methods can also be used, including addition or omission of one or more steps.

The readouts for this study are vascular leakage in the lung and edema formation in the lung. The following animal groups are used for this study:
Group 1 (Control), 4 mice (2 males and 2 females, 2-month old).
Group 2 (SARS-CoV-2, 5×10⁴ pfu/mice for 7 days), 4 mice (2 males and 2 females, 2-month old).
Group 3 (sACE2₂.v2.4-IgG1 administered IV (10 mg/kg) or IT (2 mg/kg) or by inhalation hours prior to SARS-CoV-2 infection, 5×10⁴ pfu/mice for 7 days), 4 mice (2 males and 2 females, 2-month old). This group assesses pre-exposure prophylaxis.
Group 4 (sACE2₂.v2.4-IgG1 administered IV (10 mg/kg) or IT (2 mg/kg) or by inhalation after SARS-CoV-2 infection, 5×10⁴ pfu/mice for 7 days), 4 mice (2 males and 2 females, 2-month old). This group assesses post-infection therapy.

Mice are administered sACE2₂.v2.4-IgG1 polypeptide by one of several methods (e.g., IV, IT, inhalation) and infected with SARS-CoV-2 via the airway to mimic human lung infection.

It is expected that sACE2₂.v2.4-IgG1 will reduce SARS-CoV-2-induced lung vascular leak and reduce edema formation, which are the primary causes of respiratory failure and death in COVID-19 patients.

### Example 8

This example describes a study to investigate whether sACE2₂.v2.4-IgG1 exhibits a protective and/or therapeutic benefit against SARS-CoV-2-induced lung vascular injury and long term fibrosis in a mouse model of COVID-19. While particular methods are provided, one of skill in the art will recognize that methods that deviate from these specific methods can also be used, including addition or omission of one or more steps.

The readouts for this study are H&E staining, Masson trichrome and Sirius red staining, MPO assay, and protein lysates to assess signaling shifts and inflammatory pathology. The following animal groups are used for this study:
Group 1 (Control), 4 mice (2 males and 2 females, 2-month old).
Group 2 (SARS-CoV-2, 5×10⁴ pfu/mice for 7 days), 4 mice (2 males and 2 females, 2-month old).
Group 3 (sACE2₂.v2.4-IgG1 administered IV (10 mg/kg) or IT (2 mg/kg) or by inhalation prior to SARS-CoV-2 infection, 5×10⁴ pfu/mice for 7 days), 4 mice (2 males and 2 females, 2-month old). This group assesses pre-exposure prophylaxis.
Group 4 (sACE2₂.v2.4-IgG1 administered IV (10 mg/kg) or IT (2 mg/kg) or by inhalation after SARS-CoV-2 infection, 5×10⁴ pfu/mice for 7 days), 4 mice (2 males and 2 females, 2-month old). This group assesses post-infection therapy.

It is expected that sACE2₂.v2.4-IgG1 will reduce inflammatory injury and fibrosis in this mouse model of COVID-19.

### Example 9

This example describes a study to investigate whether sACE2₂.v2.4 (with and without fusion to IgG1 Fc) blocks the spike proteins of highly transmissible SARS-CoV-2 variants. Mutants of SARS-CoV-2 have emerged that show increased transmission and possibly increased virulence. The virus variants of concern as of March, 2021 are B.1.351 originating from South Africa (Tegally et al., medRxiv, in press, doi:10.1101/2020.12.21.20248640), P.1 from Brazil, and B.1.1.7 from England (Leung et al., Eurosurveillance 26, 2021, doi:10.2807/1560-7917.ES.2020.26.1.2002106; Volz et al., medRxiv, in press, doi:10.1101/2020.12.30.20249034). All three virus variants share the N501Y mutation in S, which increases monovalent affinity for wild type ACE2 by 20-fold (Example 4—Table 6). The high affinity v2.4 ACE2 derivative also binds with increased affinity (Example 4—Table 6). This study tests the apparent monovalent affinity and avid binding of dimeric sACE2₂-IgG1 (wild type and v2.4) with full-length S variants from the P.1, B.1.1.7, and B.1.351 lineages.

S proteins are expressed in human Expi293F cells with N-terminal c-myc tags for measuring surface expression with a fluorescent anti-myc antibody and flow cytometry. Cells are incubated with a dilution series of sACE2-8his and sACE2.v2.4-8his (monomer: ACE2 residues 19-615), washed, and bound protein is measured by flow cytometry using anti-his fluorescent antibody staining. Cells are also incubated with a dilution series of sACE2₂-IgG1 and sACE2₂.v2.4-IgG1 (dimer: ACE2 residues 19-732), washed, and bound protein is measured by flow cytometry using an anti-human IgG1 fluorescent antibody. Based on the previously described deep mutagenesis (Example 4), it is expected that the results will confirm that highly transmissible virus variants remain susceptible to tight binding by the engineered v2.4 derivative of sACE2.

In view of the many possible embodiments to which the principles of the disclosed subject matter may be applied, it should be recognized that the illustrated embodiments are only preferred examples of the disclosure and should not be taken as limiting the scope of the disclosure. Rather, the scope of the disclosure is defined by the following claims. We therefore claim all that comes within the scope and spirit of these claims.

1. A modified angiotensin-converting enzyme 2 (ACE2) polypeptide, comprising a human ACE2 or a fragment thereof, wherein the polypeptide comprises at least one amino acid substitution relative to wild-type human ACE2 of SEQ ID NO: 1, and has increased binding to the S protein of severe acute respiratory syndrome coronavirus 2 (SARS-CoV-2) relative to wild-type human ACE2.

2. The modified polypeptide of claim 1, wherein:
the at least one amino acid substitution is a substitution selected from the group consisting of T27Y, L79T, N330Y, S19P, E23F, Q24T, A25V, K26I, K26A, K26D, T27M, T27L, T27A, T27D, T27K, T27H, T27W, T27F, T27C, L29F, D30I, D30E, K31W, K31Y, N33D, H34V, H34A, H34S, H34P, E35V, E35C, L39K, L39R, F40D, F40R, Y41R, Q42M, Q42L, Q42I, Q42V, Q42K, Q42C, A65W, W69I, W69V, I69T, I69K, F72Y, E75A, E75S, E75T, E75K, E75R, E75W, E75G, Q76M, Q76I, Q76V, Q76T, Q76R, Q76Y, L79I, L79V, L79W, L79Y, L79F, L79P, M82C, Q89I, Q89D, Q89P, N90M, N90L, N90I, N90V, N90A, N90S, N90T, N90Q, N90D, N90E, N90K, N90R, N90H, N90W, N90Y, N90F, N90P, N90G, N90C, L91P, T92M, T92L, T92I, T92V, T92A, T92N, T92Q, T92D, T92E, T92K, T92R, T92H, T92W, T92Y, T92F, T92P, T92G, T92C, T324E, T324P, Q325P, N330L, N330H, N330W, N330F, L351F, A386L, A386I, P389D, R393K and R518G, with reference to SEQ ID NO: li
the at least one amino acid substitution is a substitution selected from the group consisting of T27Y, L79T, N330Y, S19P, A25V, T27M, T27L, T27A, T27D, T27H, T27W, T27F, T27C, D30E, K31W, H34V, H34A, H34P, L39K, L39R, Q42M, Q42L, Q42C, W69V, F72Y, E75K, E75R, Q76V, Q76T, L79I, L79V, L79W, L79Y, L79F, Q89P, N90M, N90L, N90I, N90V, N90A, N90S, N90T, N900, N90D, N90E, N90K, N90R, N90H, N90P, N90G, N90C, L91P, T92M, T92L, T92I, T92V, T92A, T92N, T92Q, T92D, T92E, T92K, T92R, T92H, T92W, T92Y, T92F, T92P, T92G, T92C, T324E, T324P, Q325P, N330L, N330H, N330W, N330F, L351F and A386L, with reference to SEQ ID NO: 1:
wherein the at least one amino acid substitution is a substitution selected from the group consisting of T27Y, L79T, N330Y, A25V, T27M, T27L, K31W, H34V, H34A, H34P, Q42L, Q42C, L79I, L79V, L79W, L79Y, L79F, N90A, N90S, N90T, N900, N90E, N90H, L91P, T92M, T92L, T92I, T92V, T92N, T92Q, T92D, T92E, T92R, T92H, T92W, T92Y, T92F, T92G, T92C, T324P, Q325P, N330H, N330W, N330F and A386L, with reference to SEQ ID NO: 1:
the at least one amino acid substitution is at residue 19, 23, 24, 25, 26, 27, 29, 30, 31, 33, 34, 35, 39, 40, 41, 42, 65, 69, 72, 75, 76, 79, 82, 89, 90, 91, 92, 324, 325, 330, 351, 386, 389, 393 or 518 of human ACE2 of SEQ ID NO: 1; and/or
the at least one amino acid substitution is selected from the group consisting of T27Y, L79T, N330Y, S19P, A25V, K26D, L29F, N33D, L39R, F40D, W69V, F72Y, Q76T, Q89P, L91P, T324P, T324E, Q325P, R518G, L351F, A386L, Q24T, T27H, D30E, K31Y, H34A, Y41R, Q42L, Q42K, E75K, L79V, N900, T920, N330H and R393K, with reference to SEQ ID NO: 1.

3-6. (canceled)

7. The modified polypeptide of claim 1, wherein the at least one amino acid substitution removes the glycosylation motif at residues N90, L91 and T92 of human ACE2 of SEQ ID NO: 1.

8. The modified polypeptide of claim 1, comprising:
T27Y, L79T, and N330Y amino acid substitutions;
H34A, T92Q, Q325P, and A386L amino acid substitutions;
T27Y, L79T, N330Y, and A386L amino acid substitutions;
L79T, N330Y, and A386L amino acid substitutions;
T27Y, N330Y, and A386L amino acid substitutions;
T27Y, L79T, and A386L amino acid substitutions;
A25V, T27Y, T92Q, Q325P, and A386L amino acid substitutions;
H34A, L79T, N330Y, and A386L amino acid substitutions;
A25V, T92Q, and A386L amino acid substitutions; or
T27Y, Q42L, L79T, T92Q, Q325P, N330Y, and A386L amino acid substitutions,
wherein the amino acid substitutions are with reference to SEQ ID NO: 1.

9. The modified polypeptide of claim 1, having a single amino acid substitution relative to human ACE2 of SEQ ID NO: 1.

10. The modified polypeptide of claim 1, comprising full-length human ACE2 and comprising at least one amino acid substitution relative to wild-type human ACE2.

11-12. (canceled)

13. The modified polypeptide of claim 1, wherein the polypeptide consists of a fragment of human ACE2.

14. The modified polypeptide of claim 13, wherein the fragment of human ACE2 is an extracellular fragment.

15. The modified polypeptide of claim 14, wherein the extracellular fragment corresponds to residues 19 to 615 of human ACE2 of SEQ ID NO: 1 or residues 20 to 615 of human ACE2 of SEQ ID NO: 1.

16-18. (canceled)

19. The modified polypeptide of claim 13, wherein the fragment corresponds to residues 1-732, 19-732 or 19-740 of human ACE2 of SEQ ID NO: 1.

20. (canceled)

21. The modified polypeptide of claim 19, wherein the amino acid sequence of the fragment consists of SEQ ID NO: 10.

22. The modified polypeptide of claim 1, wherein the polypeptide forms a dimer.

23. A fusion protein comprising the modified polypeptide of claim 1 and a heterologous polypeptide.

24. The fusion protein of claim 23, wherein the heterologous polypeptide is an Fc protein, a fluorescent protein, an enzyme, an antibody or antigen-binding protein, a cytokine, a cellular ligand or receptor, or serum albumin.

25-26. (canceled)

27. The fusion protein of claim 23, wherein the amino acid sequence of the fusion protein comprises or consists of SEQ ID NO: 11.

28. (canceled)

29. A composition comprising the modified polypeptide of claim 1, and a pharmaceutically acceptable carrier.

30. The composition of claim 29, formulated for intratracheal or inhalation administration.

31. An in vitro method of inhibiting replication of a coronavirus (CoV), comprising contacting the CoV with the modified polypeptide of claim 1.

32. A method of inhibiting coronavirus (CoV) replication and/or spread in a subject, comprising administering to the subject a therapeutically or prophylactically effective amount of the modified polypeptide of claim 1, thereby inhibiting CoV replication and/or spread in the subject.

33. The method of claim 32, comprising administering the modified polypeptide intravenously, intratracheally or by inhalation.

34. (canceled)

35. A nucleic acid molecule encoding the modified polypeptide of claim 1.

36. A vector comprising the nucleic acid molecule of claim 35.

37. A composition comprising the nucleic acid molecule of claim 35 and a pharmaceutically acceptable carrier.

38. A method of inhibiting coronavirus (CoV) replication and/or spread in a subject, comprising administering to the subject a therapeutically or prophylactically effective amount of the nucleic acid molecule of claim 35, thereby inhibiting CoV replication and/or spread in the subject.

39. (canceled)

40. The method of claim 38, wherein the nucleic acid molecule is administered by intravenous, intratracheal or inhalation administration.

41. A method of detecting a coronavirus (CoV) in a biological sample, comprising:
contacting the biological sample with the modified polypeptide of claim 1; and
detecting binding of the modified polypeptide to the biological sample, thereby detecting the CoV in the biological sample.

42. (canceled)

43. The method of claim 32, wherein the coronavirus is a human coronavirus or a zoonotic coronavirus.

44. The method of claim 43, wherein:
the human coronavirus is severe acute respiratory syndrome coronavirus (SARS-CoV), SARS-CoV-2, Middle East respiratory syndrome coronavirus (MERS-CoV), human coronavirus HKU1 (HKU1-CoV), human coronavirus OC43 (OC43-CoV), human coronavirus 229E (229E-CoV), or human coronavirus NL63 (NL63-CoV); or
the zoonotic coronavirus is a bat coronavirus or a rodent coronavirus.

45-47. (canceled)

48. A kit comprising the modified polypeptide of claim 1 bound to a solid support.


SEQUENCE LISTING (11 sequences)

SEQ ID NO: 1 - protein, 805 aa, Homo sapiens [sequence omitted]

SEQ ID NO: 2 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 196 aa, SARS-CoV-2 Wuhan
TNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPK

SEQ ID NO: 4 - protein, 195 aa, SARS-CoV-1 Urbani
TNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPK

SEQ ID NO: 5 - protein, 195 aa, Rs4084-CoV
TNLCPFGEVFNATTFPSVYAWERKRISNCVADYSILYNSTSFSTFKCYGVSATKLNDLCFSNVYADSFVVKGDDVRQIAPGQTGVIADYNYKLPDDFLGCVLAWNTNSKDSSTSGNYNYLYRWVRRSKLNPYERDLSNDIYSPGGQSCSAVGPNCYNPLRPYGFFTTAGVGHQPYRVVVLSFELLNAPATVCGPK

SEQ ID NO: 6 - protein, 195 aa, RsSHC014-CoV
TNLCPFGEVFNATTFPSVYAWERKRISNCVADYSVLYNSTSFSTFKCYGVSATKLNDLCFSNVYADSFVVKGDDVRQIAPGQTGVIADYNYKLPDDFLGCVLAWNTNSKDSSTSGNYNYLYRWVRRSKLNPYERDLSNDIYSPGGQSCSAVGPNCYNPLRPYGFFTTAGVGHQPYRVVVLSFELLNAPATVCGPK

SEQ ID NO: 7 - protein, 195 aa, Rs4231-CoV
TNLCPFGEVFNATTFPSVYAWERKRISNCVADYSVLYNSTSFSTFKCYGVSATKLNDLCFSNVYADSFVVKGDDVRQIAPGQTGVIADYNYKLPDDFLGCVLAWNTNSKDSSTSGNYNYLYRWVRRSKLNPYERDLSNDIYSPGGQSCSAIGPNCYNPLRPYGFFTTAGVGHQPYRVVVLSFELLNAPATVCGPK

SEQ ID NO: 8 - protein, 195 aa, LYRa11-CoV
TNLCPFGEVFNATTFPSVYAWERKRISNCVADYSVLYNSTSFSTFKCYGVSAIKLNDLCFSNVYADSFVVKGDDVRQIAPGQTGVIADYNYKLPDDFMGCVLAWNTRNIDATSSGNFNYKYRSLRHGKLRPFERDISNVPFSPDGKPCTPPAFNCYWPLNDYGFYTTNGIGYQPYRVVVLSFELLNAPATVCGPK

SEQ ID NO: 9 - protein, 195 aa, Rs7327-CoV
TNLCPFGEVFNATTFPSVYAWERKRISNCVADYSVLYNSTSFSTFKCYGVSATKLNDLCFSNVYADSFVVKGDDVRQIAPGQTGVIADYNYKLPDDFMGCVLAWNTRNIDATSTGNYNYKYRSLRHGKLRPFERDISNVPFSPDGKPCTPPAFNCYWPLNDYGFFTTNGIGYQPYRVVVLSFELLNAPATVCGPK

SEQ ID NO: 10 - protein, 714 aa, Artificial Sequence
  Synthetic polypeptide
STIEEQAKYFLDKFNHEAEDLFYQSSLASWNYNTNITEENVQNMNNAGDKWSAFLKEQSTTAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLG

SEQ ID NO: 11 - protein, 942 aa, Artificial Sequence
  Synthetic polypeptide
STIEEQAKYFLDKFNHEAEDLFYQSSLASWNYNTNITEENVQNMNNAGDKWSAFLKEQSTTAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK